(19) United States
(12) Patent Application Publication
(10) Pub. No.: US 2021/0139883 A1
(43) Pub. Date: May 13, 2021

(54) NEW ATTENUATED STRAINS OF APICOMPLEXA AND THEIR USE AS ANTIGEN VECTORS FOR THE PREVENTION OF INFECTIOUS DISEASES

(71) Applicants: UNIVERSITE DE TOURS, TOURS CEDEX 1 (FR); INSTITUT NATIONAL DE LA RECHERCHE AGRONOMIQUE, PARIS CEDEX 07 (FR)

(72) Inventors: Anne-France BOUSSEMART-PROUVOST, MOUVAUX (FR); Edouard SECHE, OLIVET (FR); Thi Kim Chi NGUYEN, DIJON (FR)

(21) Appl. No.: 16/628,216

(22) PCT Filed: Jul. 3, 2018
(86) PCT No.: PCT/FR2018/051660

§ 371 Date: Jan. 2, 2020

(30) Foreign Application Priority Data
Jul. 3, 2017 (FR) 17/56276

(51) Int. Cl. C12N 15/10; C12N 15/66; A61K 39/002; C12N 1/36

(52) U.S. Cl. CPC C12N 15/102; C12N 15/66; A61K 2039/522; C12N 1/36; A61K 39/002

(57) ABSTRACT

Disclosed is a mutant strain of Sarcocystidae in which the two genes mic1 and mic3 are deleted containing two specific recombination sites of an enzyme allowing a specific recombination, in particular Cre-recombinase, the specific recombination sites being at the respective locus of each of the deleted genes, the specific recombination site of the enzyme allowing specific recombination, in particular of the Cre-recombinase, at the locus of the deleted mic1 gene being different from that of the deleted mic3 gene.

This invention concerns new attenuated strains of apicomplexes and their use as antigen carriers for the prevention of infectious diseases.

Apicomplexes are mainly obligatory intracellular parasites that have a life cycle that can involve several hosts. The phylum of these parasites is subdivided into several families. Toxoplasma gondii (T. gondii) belongs to the Sarcocystidae family. The cat, the definitive host, excretes the parasite into the environment as oocysts. Intermediate (i.e. all homeotherms) and definitive hosts can become infected by ingesting oocysts from food. The parasite then transforms into tachyzoites that spread throughout the body and, under pressure from the immune system, encyst with a preferential tropism for the central nervous system, retina or muscles. The ingestion of encysted tissues is the second cause of contamination of definitive and intermediate hosts.

Recently, an attenuated live strain of Toxoplasma gondii, the parasite responsible for toxoplasmosis, was developed by invalidation of two genes encoding TgMIC1 and TgMIC3 proteins (Cérède et al., 2005, J. Exp. Med., 201(3): 453-63). This strain, called Toxo tgmic1-3 KO, generates a strong and specific immune response against Toxoplasma gondii and prevents the effects of subsequent infection in mice (Ismael et al., 2006, J. Infect. Say. 194(8): 1176-83) and in sheep (Mévelec et al., 2010, Vet. Res., 41(4):49). Neospora caninum (N. caninum) is an intracellular parasite responsible for neosporosis. It also belongs to the Sarcocystidae family. The life cycle of Neospora caninum is very similar to that of T. gondii with two distinct phases: a sexual phase in the final host (i.e. canids and dogs in particular) which leads to the production of oocysts eliminated in the faeces and an asexual phase in an intermediate host (i.e. sheep, goats, cattle, equidae, etc.) which leads to the production of tachyzoites then cysts containing bradyzoites.

More recently, an attenuated live strain of Neospora caninum, the Neo ncmic1-3 KO strain, has been obtained and has been invalidated for the ncmic1 and ncmic3 genes by homologous recombination. It has been shown that this mutant strain has infectious and immunogenic properties that provide mammals with vaccine protection against the harmful effects of neosporosis.

Parasites of the Sarcocystidae family such as Toxoplasma gondii and Neospora caninum can be used to express heterologous proteins from other parasites such as Plasmodium spp, Cryptosporidium parvum or Leishmania spp.

For example, the wild strain RH of Toxoplasma gondii was used as a vector for the CSP antigen (Protein Circum Sporozoite) of Plasmodium knowlesi (Di Cristina et al., 1999, Infect Immun., 67(4): 1677-82). After random integration of the sequence of interest, the recombinant strain was inoculated into Rhesus monkeys and induced in animals a humoral immune response specific for the CSP protein. In the thermosensitive strain is-4 HXGPRT−/− of Toxoplasma gondii was used as a vector for the CSP antigen of Plasmodium yoelii (Charest et al., 2000, J. Immunol., 165(4): 2084-92). After random integration of the sequence of interest, the recombinant parasites were inoculated into the mouse inducing a humoral immune response specific for CSP antigen but insufficient to induce protection against infection with Plasmodium yoelii.

A strain of Toxoplasma gondii was also used to express the genes gp40, gp15 and the precursor gp40/gp15 of Cryptosporidium parvum, the parasite responsible for cryptosporidiosis (O'Connor et al., Infect. Immun. 2003 71(10):6027-35; O'Connor et al. 2007, Mol Biochem Parasitol, 152(2):148-58). The genes gp40/gp15, gp40 and gp15 were cloned, placed under the control of a T. gondii promoter and randomly integrated into the parasite genome. The authors showed that recombinant parasites expressed proteins of interest and that the glycosylation of the GP40 protein expressed by T. gondii was similar to the glycosylation of the native protein. However, the authors demonstrated that cleavage of the pre-protein GP40/GP15 was ineffective in tachyzoites although cleavage enzymes are present in the parasite T. gondii. Another team also investigated the use of Toxoplasma gondii as an antigen vector of Cryptosporidium parvum and in particular the immunodominant surface protein P23 (Shirafuji et al., 2005, J. Parasitol., 91(2):476-9). The molecular weight and antigenic properties of recombinant P23 are similar to those of native protein and mice immunized with lysed tachyzoites expressing P23 produce neutralizing antibodies against C. parvum.

Finally, T. gondii was used as an expression vector for the Kmp11 Leishmania antigen. The recombinant strain obtained allows significant protection of the animals during a challenge with L. major (Ramirez et al., 2001, Vaccine, 20:455-61).

Neospora caninum has also been used as a vector of heterologous antigens and a recombinant strain of N. caninum stably expressing the SAG1 antigen of T. gondii has been constructed (Zhang et al., 2010, Vaccine, 60(1):105-7). The expression level, molecular weight and antigenic properties of the SAG1 protein expressed in N. caninum are similar to those of the native SAG1 protein and immune mice produce a Th1-type immune response specific for SAG1 of T. gondii and are protected against a lethal challenge with T. gondii.

The Cre/LoxP system has been used in the activation or inactivation strategies of mammalian cell genes (Fukushige and Sauer, 1992, PNAS, 89(17): 7905-9) or transgenic mice (Tsien et al., 1996, Cell, 87(7):1317-26.).

Cre Recombinase is an enzyme derived from bacteriophage P1 (Sternberg and Hamilton, 1981, J. Mol. Biol. 150(4): 487-507) of the integrases family which recognize very specific sites and allow recombination between two identical sites. The restriction site for Cre Recombinase is the LoxP site, a 34 base pair nucleotide sequence (SEQ ID NO: 12) that includes two small sequences of 13 repeated and inverted base pairs and a spacer region (in bold) of 8 base pairs. Several mutants of the LoxP site are described and 3 sites have been identified as incompatible with each other (Livet et al., 2007, Nature, 450(7166): 56-62). These are the LoxP (SEQ ID NO: 12), LoxN (SEQ ID NO: 5) and Lox2272 (SEQ ID NO: 68) sites.

The properties of Cre recombinase are multiple and can be used for (FIG. 1):
Delete a DNA sequence present between two identical Lox sites and with the same orientation,
Invert a DNA sequence present between two identical Lox sites and with the opposite orientation,
Integrate at a LoxP or LoxN site a DNA sequence containing the LoxP or LoxN site.

In Toxoplasma gondii, the Cre/Lox system was first used in 1999 (Brecht et al., 1999, Gene, 234(2):239-47). Following the random insertion of a reporter gene framed by LoxP sites oriented in an identical direction, the action of Cre Recombinase allowed the deletion of the reporter gene and the formation of a LoxP scar. Recombinase Cre was then used to integrate a new heterologous transgene into this LoxP scar. More recently; the Cre/LoxP system has been used to produce a deleted strain of Toxoplasma gondii for the morn1 gene (Heaslip et al., 2010, PloS Pathog, 6(2): e1000754). Finally, KO (knockout) strains of T. gondii have recently been created using a dimerizable form of inducible Recombinase Cre only after the addition of a ligand: rapamycin (Andenmatten et al., 2013, Nat. Methods, 10(2): 125-7; Rugarabamu et al., 2015, Mol. Microbiol, 97(2): 244-62).

This invention concerns new attenuated strains of Sarcocystidae (Toxoplasma gondii and Neospora caninum).

This invention also concerns the use of new attenuated strains of Sarcocystidae (Toxoplasma gondii and Neospora caninum) as an antigen vector for the prevention of infectious diseases.

This invention concerns a mutant strain of Sarcocystidae in which at least one of the genes mic1 or mic3 is deleted, containing a specific recombination site of an enzyme allowing specific recombination, at the locus of said at least one deleted gene, and in the case where both mic1 and mic3 genes are deleted, the specific recombination site of the enzyme allowing specific recombination at the locus of the deleted mic1 gene is potentially different from that at the locus of the deleted mic3 gene.

Enzyme allowing specific recombination” means enzyme catalysing DNA recombination in a defined sense between specific sites determined by sequences specific to each enzyme. In particular, they allow the excision, insertion, inversion or translocation of a nucleotide sequence flanked by specific sites.

Examples of enzymes that allow specific recombination include Cre recombinase, FLP recombinase, Tre recombinase, RecA proteins and Hin recombinase (bacteria).

The mic1 and mic3 genes are used to code the MIC1 and MIC3 proteins. They are proteins of micronemes, the secretory organelles of Apicomplexes that play a central role in the recognition and adherence to host cells.

In a particular mode of production, the enzyme allowing a specific recombination is cre-recombinase. Thus, in this case, the mutant strain of Sarcocystidae in which at least one of the mic1 or mic3 genes is deleted contains a specific recombination site of the cre-recombinase at the locus of said at least one deleted gene.

and in the case where both mic1 and mic3 genes are deleted, the specific recombination site of the cre-recombinase at the locus of the deleted mic1 gene is different from that at the locus of the deleted mic3 gene.

“Gene deletion” refers to the deletion of the entire coding sequence (introns and exons), the deletion of the promoter region and the deletion of the untranslated transcribed regions 5′ and 3′, known as 5′ and 3′ UTR, the term “gene” referring to the promoter region (also known as promoter), the coding sequence and the regions 5′ and 3′ UTR.

Cre recombinase is a topoisomerase derived from the bacteriophage P1, this enzyme is functional in parasites. The possible use of several Lox sites that do not interact with each other makes it possible to consider several deletions.

Examples of specific recombination sites of Cre-recombinase (Lox sites) include, but are not limited to, LoxP, LoxN, Lox2272, Lox71, Lox66, Lox511, Lox5171 and LoxM2.

The present invention concerns a mutant strain of Sarcocystidae in which the two genes mic1 and mic3 are deleted containing two specific recombination sites of an enzyme allowing a specific recombination, in particular Cre-recombinase, said specific recombination sites being at the respective locus of each of said deleted genes, the specific recombination site of the enzyme allowing specific recombination, in particular of the Cre-recombinase, at the locus of the deleted mic1 gene being different from that of the deleted mic3 gene.

According to a particular method of implementation, the present invention concerns a mutant strain of Sarcocystidae in which the said enzyme allowing specific recombination is Cre-recombinase and in which, in the case where the two genes mic1 and mic3 are deleted, the specific recombination site of the enzyme allowing specific recombination at the locus of the deleted mic1 gene is different from that at the locus of the deleted mic3 gene.

According to a particular method of production, the said mutant strain of Sarcocystidae is a strain of the genus Toxoplasma spp.

This genus includes between the species Toxoplasma gondii.

According to a particular method of production, the said mutant strain of Sarcocystidae is a strain of the species Toxoplasma gondii.

According to a particular method of production, the said mutant strain of Sarcocystidae is a strain of the genus Toxoplasma spp. in particular a strain of the species Toxoplasma gondii.

According to another particular method of production, the said mutant strain of Sarcocystidae is a strain of the genus Neospora spp.

This genus includes, among others, the following species: Neospora caninum, Neospora hughesi.

According to a particular method of production, the said mutant strain of Sarcocystidae is a strain of the species Neospora caninum.

According to a particular method of production, the said mutant strain of Sarcocystidae is a strain of the genus Neospora spp., in particular a strain of the species Neospora caninum.

In a particular mode of realization, the present invention concerns a mutant strain of Sarcocystidae in which both mic 1 and mic 3 genes are deleted, and which contains a specific recombination site of an enzyme allowing specific recombination, in particular Cre-recombinase, at the locus of the deleted mic 1 gene, and a specific recombination site of the enzyme allowing specific recombination, in particular Cre-recombinase, at the locus of the deleted mic3 gene, the locus-specific recombination site of the deleted mic1 gene being different from the locus-specific recombination site of the deleted mic3 gene.

In the case where the enzyme allowing specific recombination is Cre-recombinase, said mutant Sarcocystidae strain in which both mic 1 and mic 3 genes are deleted, contains a specific recombination site of Cre-recombinase at the locus of the deleted mic 1 gene, and a specific recombination site of Cre-recombinase at the locus of the deleted mic3 gene, the specific recombination site of the Cre-recombinase at the locus of the deleted mic1 gene being different from that at the locus of the deleted mic3 gene.

Thus, according to a particular method of implementation, the present invention concerns a mutant strain of Sarcocystidae in which the two genes mic 1 and mic 3 are deleted, and containing two sites of specific recombination of an enzyme allowing specific recombination, in particular Cre-recombinase, each of the two sites being respectively at the locus of each of the said deleted genes, the specific recombination site of the enzyme allowing a specific recombination, in particular of the Cre-recombinase, at the locus of the deleted mic1 gene being different from that of the deleted mic3 gene.

In a particular mode of realization, the present invention concerns a mutant strain of Sarcocystidae in which both mic 1 and mic 3 genes are deleted, and which contains a specific recombination site of an enzyme allowing specific recombination, in particular Cre-recombinase, at the locus of the deleted mic 1 gene, and a specific recombination site of the enzyme allowing specific recombination, in particular Cre-recombinase, at the locus of the deleted mic3 gene,

the locus-specific recombination site of the deleted mic1 gene being different from the locus-specific recombination site of the deleted mic3 gene,

and said strain not containing heterologous DNA, other than those corresponding to the specific recombination sites of the enzyme allowing specific recombination, in particular Cre-recombinase, at the respective locus of each of the said deleted genes.

By “not containing heterologous DNA other than heterologous DNA corresponding to the specific recombination sites of the enzyme allowing specific recombination”, it is meant that the strain may contain, as the only heterologous DNA, one or more sequences corresponding to a specific recombination site of an enzyme allowing specific recombination.

In the case where the enzyme allowing a specific recombination is Cre-recombinase, said mutant Sarcocystidae strain in which both mic 1 and mic 3 genes are deleted, contains a specific recombination site of the deleted mic 1 gene at the locus of the deleted mic 1 gene, and a specific recombination site of the deleted mic3 gene at the locus of the deleted mic3 gene,

the specific recombination site of the Cre-recombinase at the locus of the deleted mic1 gene being different from that at the locus of the deleted mic3 gene,

said strain not containing heterologous DNA, other than those corresponding to the specific recombination sites of the Cre-recombinase, at the respective locus of each of said deleted genes.

Containing heterologous DNA, other than heterologous DNA corresponding to recombination sites of cre-recombinase” means that said strain contains heterologous DNA which does not correspond to a sequence of a specific recombination site of cre-recombinase and which does not include a sequence corresponding to a specific recombination site of an enzyme allowing specific recombination.

According to a particular method of realization, the present invention concerns a mutant strain of Sarcocystidae in which the two genes mic1 and mic3 are deleted, and containing two sites of specific recombination of an enzyme allowing a specific recombination in particular Cre-recombinase, each of the two sites being respectively at the location of each of the said deleted genes, the specific recombination site of the enzyme allowing a specific recombination, in particular Cre-recombinase, at the locus of the deleted mic1 gene being different from that at the locus of the deleted mic3 gene, and not containing heterologous DNA, other than those corresponding to the specific recombination sites of an enzyme allowing a specific recombination, in particular Cre-recombinase, at the respective locus of each of the said deleted genes.

In a particular mode of realization, the present invention concerns a mutant strain of Sarcocystidae in which both mic 1 and mic 3 genes are deleted, and containing a specific recombination site of the deleted mic 1 gene at the locus of the deleted mic 1 gene, and a specific recombination site of the deleted Cre-recombinase at the locus of the deleted mic3 gene,

the specific recombination site of the Cre-recombinase at the locus of the deleted mic1 gene being different from that at the locus of the deleted mic3 gene.

said strain not containing heterologous DNA, other than those corresponding to the Cre-recombinase specific recombination sites, at the respective locus of each of said deleted genes,

and wherein each of the specific recombination sites of the Cre-recombinase at the respective locus of the deleted mic1 and mic3 genes are selected from the following sites: LoxN of SEQ ID NO: 5, LoxP of SEQ ID NO: 12 and Lox2272 of SEQ ID NO: 68, the recombination site of the Cre-recombinase at the locus of the deleted mic1 gene being different to that at the locus of the deleted mic3 gene.

In a particular mode of realization, the present invention concerns a mutant strain according to the invention, in which the two genes mic1 and mic3 are deleted, containing two sites of specific recombination of an enzyme allowing specific recombination, at the respective locus of each of the said deleted genes, the site of specific recombination of the enzyme allowing specific recombination, at the locus of the deleted mic1 gene being different from that at the locus of the deleted mic3 gene, and not containing heterologous DNA, other than heterologous DNA corresponding to the specific recombination sites of the enzyme allowing specific recombination, in particular Cre-recombinase, at the respective locus of each of the said deleted genes,

in particular said enzyme allowing specific recombination being Cre-recombinase, and the specific recombination sites of Cre-recombinase at the respective locus of each of said deleted genes being chosen from: LoxN of SEQ ID NO: 5, LoxP of SEQ ID NO: 12 and Lox2272 of SEQ ID NO: 68.

According to a particular method of implementation, the present invention concerns a mutant strain of Sarcocystidae in which the two genes mic1 and mic3 are deleted, and which does not contain heterologous DNA, other than those corresponding to the specific recombination sites of an enzyme allowing specific recombination, in particular Cre-recombinase, at the respective locus of each of the said deleted genes

and containing two enzyme-specific recombination sites allowing specific recombination, said enzyme being Cre-recombinase, each of the two sites being respectively at the locus of each of said deleted genes, said specific recombination sites being cre-recombinase specific recombination sites selected from: LoxN of SEQ ID NO: 5, LoxP of SEQ ID NO: 12 and Lox2272 of SEQ ID NO: 68, the recombination site of the Cre-recombinase at the locus of the deleted mic1 gene being different from that at the locus of the deleted mic3 gene.

In a particular mode of realization, the present invention concerns a mutant strain of Sarcocystidae in which both mic 1 and mic 3 genes are deleted, and containing a specific recombination site of an enzyme allowing specific recombination, in particular Cre-recombinase, at the locus of the deleted mic 1 gene, and a specific recombination site of the enzyme allowing specific recombination, in particular Cre-recombinase, at the locus of the deleted mic3 gene,

the locus-specific recombination site of the deleted mic1 gene being different from the locus-specific recombination site of the deleted mic3 gene,

and said strain containing DNA heterologous at the locus of the deleted mic1 gene or at the locus of the deleted mic3 gene, different from the heterologous DNA corresponding to the specific recombination sites of the enzyme allowing specific recombination, in particular Cre-recombinase, at the respective locus of each of the deleted mic1 and mic3 genes, so that:
when said heterologous DNA is inserted at the locus of the deleted mic1 gene, it is flanked:
   a first enzyme-specific recombination site allowing specific recombination, in particular Cre-recombinase, corresponding to the enzyme-specific recombination site allowing specific recombination, in particular Cre-recombinase, at the locus of the deleted mic1 gene, and
   of a second specific recombination site identical to said first specific recombination site located at the locus of the deleted mic1 gene, this second site being provided in addition to the sites already present in the strain, due to the additional specific recombination when adding heterologous DNA,
when said heterologous DNA is inserted at the locus of the deleted mic3 gene, it is flanked:
   a first enzyme-specific recombination site allowing specific recombination, in particular Cre-recombinase, corresponding to the enzyme-specific recombination site allowing specific recombination, in particular Cre-recombinase, at the locus of the deleted mic3 gene, and
   a second specific recombination site identical to said first specific recombination site located at the locus of the deleted mic3 gene,

said strain comprising the means necessary for the transcription of said heterologous DNA.

This mode of realization targets the production of heterologous RNA from said heterologous DNA in a mutant strain as described above.

By “means necessary for the transcription of said heterologous DNA”, we mean a promoter, a transcription initiation site, a TATA box, a transcription terminator.

The RNA thus transcribed may be a messenger RNA, an interfering RNA, a long non-coding RNA, a stem-loop RNA (in English “Hairpin RNA”),

According to a particular method of realization, the present invention concerns a mutant strain of Sarcocystidae in which the two genes mic1 and mic3 are deleted, and containing two sites of specific recombination of an enzyme allowing specific recombination, in particular Cre-recombinase, each of the two sites being respectively at the location of each of the said deleted genes, the site of specific recombination of the enzyme allowing specific recombination, in particular Cre-recombinase, at the locus of the deleted mic1 gene being different from that at the locus of the deleted mic3 gene, and containing DNA heterologous to the locus of the deleted mic1 gene or to the locus of the deleted mic3 gene, different from the heterologous DNA corresponding to the specific recombination sites of the enzyme allowing a specific recombination in particular Cre-recombinase, at the respective locus of each of the said deleted genes,

said heterologous DNA being flanked:
a first recombination site specific to the enzyme allowing specific recombination, in particular Cre-recombinase, corresponding to the recombination site specific to the enzyme allowing specific recombination, in particular Cre-recombinase, at the locus of the deleted mic1 gene or that of the deleted mic3 gene, and
a second specific recombination site identical to said first specific recombination site

and the means necessary for the transcription of the said heterologous DNA.

In a particular mode of realization, the present invention concerns a strain according to the invention, containing a heterologous DNA at the locus of the deleted mic1 gene or at the locus of the deleted mic3 gene, different from the heterologous DNA corresponding to the specific recombination sites of the enzyme allowing a specific recombination at the respective locus of each of the said deleted genes, said heterologous DNA being flanked:
a first enzyme-specific recombination site allowing specific recombination, corresponding to the enzyme-specific recombination site allowing specific recombination, at the locus of the deleted mic1 gene or the locus of the deleted mic3 gene and,
a second specific recombination site identical to said first specific recombination site, corresponding to the enzyme-specific recombination site allowing specific recombination, at the locus of the deleted mic1 gene or the locus of the deleted mic3 gene and,

and the means necessary for its transcription, in particular said enzyme allowing specific recombination being Cre-recombinase and the specific recombination sites of Cre-recombinase being chosen from: LoxN of SEQ ID NO: 5, LoxP of SEQ ID NO: 12 and Lox2272 of SEQ ID NO: 68.

In a particular mode of realization, the present invention concerns a mutant strain of Sarcocystidae in which both mic 1 and mic 3 genes are deleted, and containing a specific recombination site of an enzyme allowing specific recombination, in particular Cre-recombinase, at the locus of the deleted mic 1 gene, and a specific recombination site of the enzyme allowing specific recombination, in particular Cre-recombinase, at the locus of the deleted mic3 gene,

the locus-specific recombination site of the deleted mic1 gene being different from the locus-specific recombination site of the deleted mic3 gene,

said strain containing DNA heterologous to the locus of the deleted mic1 gene or to the locus of the deleted mic3 gene, other than those corresponding to the specific recombination sites of the enzyme allowing specific recombination, in particular Cre-recombinase, at the respective locus of each of the deleted mic1 and mic3 genes, so that:
when said heterologous DNA is inserted at the locus of the deleted mic1 gene, it is flanked:
   a first enzyme-specific recombination site allowing specific recombination, in particular Cre-recombinase, corresponding to the enzyme-specific recombination site allowing specific recombination, in particular Cre-recombinase, at the locus of the deleted mic1 gene, and
   a second specific recombination site identical to said first specific recombination site located at the locus of the deleted mic1 gene,
when said heterologous DNA is inserted at the locus of the deleted mic3 gene, it is flanked:
   a first enzyme-specific recombination site allowing specific recombination, in particular Cre-recombinase, corresponding to the enzyme-specific recombination site allowing specific recombination, in particular Cre-recombinase, at the locus of the deleted mic3 gene, and
   a second specific recombination site identical to said first specific recombination site located at the locus of the deleted mic3 gene,

said heterologous DNA encoding at least one protein, said mutant strain also includes the means necessary for the expression of said heterologous DNA.

This mode of realization targets the protein expression of said heterologous DNA in a mutant as described above.

In a particular mode of realization, the present invention concerns a strain according to the invention containing a heterologous DNA at the locus of the deleted mic1 gene or at the locus of the deleted mic3 gene, different from the heterologous DNA corresponding to the specific recombination sites of the enzyme allowing a recombination specific to the respective locus of each of the said deleted mic1 and mic3 genes, the said heterologous DNA coding for a protein and being flanked:
a first enzyme-specific recombination site allowing specific recombination corresponding to the enzyme-specific recombination site allowing specific recombination at the locus of the deleted mic1 gene or at the locus of the deleted mic3 gene, and
a second specific recombination site identical to said first specific recombination site, corresponding to the enzyme-specific recombination site allowing specific recombination, at the locus of the deleted mic1 gene or the locus of the deleted mic3 gene and,

and the means necessary for the protein expression of the said heterologous DNA, in particular said enzyme allowing specific recombination being Cre-recombinase and, the specific recombination sites of Cre-recombinase being chosen from: LoxN of SEQ ID NO: 5, LoxP of SEQ ID NO: 12 and Lox2272 of SEQ ID NO: 68.

According to a particular method of implementation, the present invention concerns a mutant strain of Sarcocystidae in which the two genes mic1 and mic3 are deleted, containing two sites of specific recombination of an enzyme allowing specific recombination, in particular Cre-recombinase, each of the two sites being respectively at the locus of each of the said deleted genes, the specific recombination site of the enzyme allowing a specific recombination, in particular Cre-recombinase, at the locus of the deleted mic1 gene being different from that of the deleted mic3 gene,

and containing a heterologous DNA at the locus of the deleted mic1 gene or at the locus of the deleted mic3 gene, different from the heterologous DNA corresponding to the specific recombination sites of the enzyme allowing a specific recombination, in particular Cre-recombinase, at the respective locus of each of the said deleted genes, said heterologous DNA encoding at least one protein and being flanked:
a first recombination site specific for the enzyme allowing specific recombination, in particular Cre-recombinase, corresponding to the recombination site specific for the enzyme allowing specific recombination, in particular Cre-recombinase at the locus of the deleted mic1 gene or at the locus of the deleted mic3 gene, and
a second specific recombination site identical to said first specific recombination site

and the means necessary for the expression of the said heterologous DNA.

In a particular mode of realization, the present invention concerns a mutant strain of Sarcocystidae in which both mic 1 and mic 3 genes are deleted, and containing a specific recombination site of the deleted mic 1 gene at the locus of the deleted mic 1 gene, and a specific recombination site of the deleted Cre-recombinase at the locus of the deleted mic3 gene,

the specific recombination site of the Cre-recombinase at the locus of the deleted mic1 gene (called site A) being different from that at the locus of the deleted mic3 gene (called site B), and said strain containing DNA heterologous to the locus of the deleted mic1 gene or the locus of the deleted mic3 gene, different from those corresponding to the specific recombination sites of the Cre-recombinase, to the respective locus of each of the deleted mic1 and mic3 genes, so that:
when said heterologous DNA is inserted at the locus of the deleted mic1 gene, it is flanked:
   a first Cre-recombinase-specific recombination site, corresponding to the Cre-recombinase-specific recombination site at the locus of the deleted mic1 gene (site A), and
   a second specific recombination site (site C), identical to the first specific recombination site located at the locus of the deleted mic1 gene (site A),

and the means necessary for the expression of the said heterologous DNA,
when said heterologous DNA is inserted at the locus of the deleted mic3 gene, it is flanked:
   a first enzyme-specific recombination site allowing specific recombination, in particular Cre-recombinase, corresponding to the enzyme-specific recombination site allowing specific recombination, in particular Cre-recombinase, at the locus of the deleted mic3 gene (site B), and
   a second specific recombination site (site C), identical to the first specific recombination site located at the mic3 gene (site B) deleted,

and the means necessary for the expression of the said heterologous DNA, said three specific recombination sites A, B and C being selected from the following sites: LoxN of SEQ ID NO: 5, LoxP of SEQ ID NO: 12 and Lox2272 of SEQ ID NO: 68, such that
said site A and said site B are different, and
said site C is identical to said site A or B,

it being understood that said strain comprises the elements necessary for the transcription of said heterologous DNA, or the means necessary for the expression of said heterologous DNA when said heterologous DNA encodes at least one protein.

According to a particular method of implementation, the present invention concerns a mutant strain of Sarcocystidae containing DNA heterologous at the locus of the deleted mic1 gene or at the locus of the deleted mic3 gene, different from the heterologous DNA corresponding to the specific recombination sites of an enzyme allowing specific recombination, in which said enzyme is Cre-recombinase, at the respective locus of each of the said deleted mic1 and mic3 genes,

and containing three specific recombination sites which are respectively:
site A corresponding to the specific recombination site of Cre-recombinase at the locus of the deleted mic1 gene
site B corresponding to the specific recombination site of Cre-recombinase at the locus of the deleted mic3 gene, and
the site C corresponding to said second specific recombination site which flanks said second heterologous DNA identical to said first specific recombination site, said first Cre-recombinase specific recombination site corresponding to said Cre-recombinase specific recombination site at the locus of the deleted mic1 gene (site A) or said Cre-recombinase specific recombination site at the locus of the deleted mic3 gene (site B)

said three specific recombination sites A, B and C being selected from the following sites: LoxN of SEQ ID NO: 5, LoxP of SEQ ID NO: 12 and Lox2272 of SEQ ID NO: 68, such that
said site A and said site B are different, and
said site C is identical to said site A or said site B.

In a particular mode of realization, the present invention concerns a mutant strain of Sarcocystidae in which both mic 1 and mic 3 genes are deleted, and containing a specific recombination site of the deleted mic 1 gene at the locus of the deleted mic 1 gene, and a specific recombination site of the deleted Cre-recombinase at the locus of the deleted mic3 gene,

such that the specific recombination site of the Cre-recombinase at the locus of the deleted mic1 gene (called site A) corresponds to a LoxN site SEQ ID NO: 5 and that at the locus of the deleted mic3 gene (called site B) corresponds to a LoxP site SEQ ID NO: 12,

and said strain containing DNA heterologous at the locus of the deleted mic1 gene or the locus of the deleted mic3 gene, different from the heterologous DNA corresponding to the specific recombination sites of the Cre-recombinase, at the respective locus of each of the deleted mic1 and mic3 genes, so that:
when said heterologous DNA is inserted at the locus of the deleted mic1 gene, it is flanked:
   a first Cre-recombinase specific recombination site, corresponding to the Cre-recombinase specific recombination site, at the locus of the deleted mic1 gene (site A) corresponding to a LoxN site SEQ ID NO: 5, and
   a second specific recombination site (site C) corresponding to a LoxN site SEQ ID NO: 5, identical to the first specific recombination site located at the locus of the deleted mic1 gene (site A),
when said heterologous DNA is inserted at the locus of the deleted mic3 gene, it is flanked:
   a first Cre-recombinase-specific recombination site, corresponding to a Cre-recombinase-specific recombination site, at the locus of the deleted mic3 gene (site B) corresponding to a LoxP site SEQ ID NO: 12, and
a second specific recombination site (site C) corresponding to a LoxP site SEQ ID NO: 12, identical to the first specific recombination site located at the locus of the deleted mic3 gene (site B),

it being understood that said strain comprises the elements necessary for the transcription of said heterologous DNA, or the means necessary for the expression of said heterologous DNA when said heterologous DNA encodes at least one protein.

In a particular mode of realization, the present invention concerns a mutant strain of Sarcocystidae in which both mic 1 and mic 3 genes are deleted, and containing a specific recombination site of the deleted mic 1 gene at the locus of the deleted mic 1 gene, and a specific recombination site of the deleted Cre-recombinase at the locus of the deleted mic3 gene,

such that the specific recombination site of the Cre-recombinase at the locus of the deleted mic1 gene (called site A) corresponds to a LoxP site SEQ ID NO: 12 and that at the locus of the deleted mic3 gene (called site B) corresponds to a LoxN site SEQ ID NO: 5, and said strain containing DNA heterologous at the locus of the deleted mic1 gene or the locus of the deleted mic3 gene, different from the heterologous DNA corresponding to the specific recombination sites of the Cre-recombinase, at the respective locus of each of the deleted mic1 and mic3 genes, so that:
when said heterologous DNA is inserted at the locus of the deleted mic1 gene, it is flanked:
   a first Cre-recombinase specific recombination site, corresponding to the Cre-recombinase specific recombination site, at the locus of the deleted mic1 gene (site A) corresponding to a LoxP site SEQ ID NO: 12, and
   a second specific recombination site (site C) corresponding to a LoxP site SEQ ID NO: 12, identical to the first specific recombination site located at the locus of the deleted mic1 gene (site A),
when said heterologous DNA is inserted at the locus of the deleted mic3 gene, it is flanked:
   a first Cre-recombinase-specific recombination site, corresponding to a Cre-recombinase-specific recombination site, at the locus of the deleted mic3 gene (site B) corresponding to a LoxN site SEQ ID NO: 5, and
   a second specific recombination site (site C) corresponding to a LoxN site SEQ ID NO: 5, identical to the first specific recombination site located at the locus of the deleted mic3 gene (site B),

it being understood that said strain comprises the elements necessary for the transcription of said heterologous DNA, or the means necessary for the expression of said heterologous DNA when said heterologous DNA encodes at least one protein.

According to a particular method of implementation, the present invention concerns a mutant strain of Sarcocystidae containing DNA heterologous at the locus of the deleted mic1 gene or at the locus of the deleted mic3 gene, different from the heterologous DNA corresponding to the specific recombination sites of an enzyme allowing specific recombination, in which said enzyme is Cre-recombinase, at the respective locus of each of the said deleted mic1 and mic3 genes,

and containing three specific recombination sites which are respectively:
site A corresponding to the specific recombination site of Cre-recombinase at the locus of the deleted mic1 gene
site B corresponding to the specific recombination site of Cre-recombinase at the locus of the deleted mic3 gene, and
the site C corresponding to said second specific recombination site which flanks said second heterologous DNA identical to said first specific recombination site, said first Cre-recombinase specific recombination site corresponding to said Cre-recombinase specific recombination site at the locus of the deleted mic1 gene (site A) or said Cre-recombinase specific recombination site at the locus of the deleted mic3 gene (site B)

said three specific recombination sites A, B and C being selected from the following sites: LoxN of SEQ ID NO: 5, LoxP of SEQ ID NO: 12 and Lox2272 of SEQ ID NO: 68, such as
said site A is the LoxN site of SEQ ID NO: 5
said site B is the LoxP site of SEQ ID NO: 12, and
said site C is the LoxN site of SEQ ID NO: 5, said first specific recombination site of Cre-recombinase being site A;

or such as
said site A is the LoxN site of SEQ ID NO: 5
said site B is the LoxP site of SEQ ID NO: 12, and
said site C is the LoxP site of SEQ ID NO: 12, said first specific recombination site of Cre-recombinase being site B;

or such as
said site A is the LoxP site of SEQ ID NO: 12
said site B is LoxN site of SEQ ID NO: 5, and
said site C is the LoxP site of SEQ ID NO: 12, said first specific recombination site of Cre-recombinase being site A;

or such as
said site A is the LoxP site of SEQ ID NO: 12
said site B is LoxN site of SEQ ID NO: 5, and
said site C is the LoxN site of SEQ ID NO: 5, said first specific recombination site of Cre-recombinase being site B.

According to a particular method of realization, the present invention concerns a mutant strain of Toxoplasma spp. in which both genes mic 1 and mic 3, and the gene rop16I are deleted, and which contains at the locus of each of these deleted genes a specific recombination site of an enzyme allowing a specific recombination, in particular Cre-recombinase, such as the locus-specific recombination site of the deleted mic1 gene is different from the locus-specific recombination site of the deleted mic3 gene,

and the locus-specific recombination site of the deleted rop16I gene is different from the specific recombination site at the locus of the deleted mic1 gene and the specific recombination site at the locus of the deleted mic3 gene.

The rop16 gene is used to encode the Rop16 protein, which is a rophtria protein. This protein is a threonine serine kinase.

These ROPs proteins are secreted to allow the invagination of the plasma membrane of the host cell and the formation of the parasitophoric vacuole. PORs released into the cytosol of the host cell can migrate to the surface of the parasitophoric vacuole (ROP5, ROP18, ROP2) or into the nucleus (ROP16, protein phosphatase 2C or PP2C-hn), allowing modulation of the expression of genes involved in the host's immune response.

According to a particular mode of realization, the present invention concerns a mutant strain of Sarcocystidae in which the said mutant strain is a mutant strain of Toxoplasma spp. and in which the rop16I gene is deleted,

and which contains a specific recombination site of an enzyme allowing a specific recombination, in particular Cre-recombinase, at the locus of said deleted rop16I gene, said site being different from said specific recombination site located at the locus of the deleted mic1 gene and said specific recombination site located at the locus of the deleted mic3 gene.

According to a particular method of realization, the present invention concerns a mutant strain of Toxoplasma spp. in which both genes mic 1 and mic 3, and the gene rop16I are deleted, and which contains at the locus of each of these deleted genes a specific recombination site of an enzyme allowing a specific recombination, in particular Cre-recombinase, such as

the locus-specific recombination site of the deleted mic1 gene is different from the locus-specific recombination site of the deleted mic3 gene,

and the locus-specific recombination site of the deleted rop16I gene is different from the specific recombination site at the locus of the deleted mic1 gene and the specific recombination site at the locus of the deleted mic3 gene.

and wherein said mutant strain also contains at the locus of the rop16I gene, downstream of said locus-specific recombination site of the deleted rop16I gene, a gene encoding the protein GRA15II, as well as the means necessary for the expression of said protein,

GRAs proteins are associated with the membranous nanotubular network and the membrane of the parasitophoric vacuole (Mercier et al., Int J Parasitol. 2005 July; 35(8):829-49. Review. Erratum in: Int J Parasitol. 2005 December; 35(14):1611-2). They participate in the exchange of nutrients between the parasite and the organelles (mitochondria and endoplasmic reticulum) of the host cell (Sibley, Immunol Rev. 2011 March; 240(1):72-91).

Recently, some proteins in dense granules, including GRA15, have been identified as proteins that play a role in modulating/controlling the host cell's immune response. GRA15 has a polymorphism according to the typology of the parasite (I, II, III . . . ).

In this mode of realization, the said gene encoding the GRA15II protein at the locus of the deleted rop16I gene is therefore flanked upstream by the said specific recombination site of an enzyme allowing a specific recombination, in particular Cre-recombinase, at the locus of the said deleted rop16I gene.

According to a particular method of realization, the present invention concerns a mutant strain of Sarcocystidae in which the said mutant strain is a mutant strain of Toxoplasma spp, and in which the rop16I gene is deleted and contains a recombination site specific for an enzyme allowing a specific recombination, in particular Cre-recombinase, at the locus of said deleted rop16I gene,

said site being different from said specific recombination site located at the locus of the deleted midi gene and said specific recombination site located at the locus of the deleted mic3 gene,

and said strain comprising a gene encoding the protein GRA15II as well as the means necessary for the expression of said protein at the locus of said deleted rop16I gene, said gene encoding the GRA15II protein at the locus of said deleted rop16I gene, being flanked upstream by said enzyme-specific recombination site allowing specific recombination, in particular Cre-recombinase, at the locus of said deleted rop16I gene.

According to a particular method of realization, the present invention concerns a mutant strain of Toxoplasma spp. in which both genes mic 1 and mic 3, and the gene rop16I are deleted,

and which contains at the locus of each of these deleted genes a specific recombination site of the Cre-recombinase, such as

the specific recombination site of Cre-recombinase at the locus of the deleted mic1 gene is different from the specific recombination site at the locus of the deleted mic3 gene,

and the specific recombination site of Cre-recombinase at the locus of the deleted rop16I gene is different from the specific recombination site of Cre-recombinase at the locus of the deleted mic1 gene and the specific recombination site of Cre-recombinase at the locus of the deleted mic3 gene

said specific recombination site of the Cre-recombinase at the locus of the deleted rop16I gene is selected from the following sites: LoxN of SEQ ID NO: 5, LoxP of SEQ ID NO: 12 and Lox2272 of SEQ ID NO: 68.

According to a particular method of realization, the present invention concerns a mutant strain of Toxoplasma spp. in which both genes mic 1 and mic 3, and the gene rop16I are deleted, and which contains at the locus of each of these deleted genes a specific recombination site of the Cre-recombinase, such as

the specific recombination site of Cre-recombinase at the locus of the deleted mic1 gene is different from the specific recombination site at the locus of the deleted mic3 gene,

and the specific recombination site of Cre-recombinase at the locus of the deleted rop16I gene is different from the specific recombination site of Cre-recombinase at the locus of the deleted mic1 gene and the specific recombination site of Cre-recombinase at the locus of the deleted mic3 gene

said specific recombination sites of Cre-recombinase at the location of the deleted mid, mic3 and rop16I genes are selected from the following sites: LoxN of SEQ ID NO: 5, LoxP of SEQ ID NO: 12 and Lox2272 of SEQ ID NO: 68,

such that these three sites are different from each other.

According to a particular method of realization, the present invention concerns a mutant strain of Toxoplasma spp. in which both genes mic 1 and mic 3, and the gene rop16I are deleted, and which contains at the locus of each of these deleted genes a specific recombination site of the Cre-recombinase, such as

the specific recombination site of Cre-recombinase at the locus of the deleted mic1 gene is different from the specific recombination site at the locus of the deleted mic3 gene,

and the specific recombination site of Cre-recombinase at the locus of the deleted rop16I gene is different from the specific recombination site of Cre-recombinase at the locus of the deleted mic1 gene and the specific recombination site of Cre-recombinase at the locus of the deleted mic3 gene,

and wherein said mutant strain also contains at the locus of the rop16I gene, downstream of said locus-specific recombination site of the deleted rop16I gene, a gene encoding the protein GRA15II as well as the means necessary for the expression of said protein,

said specific recombination site of the Cre-recombinase at the locus of the deleted rop16I gene is selected from the following sites: LoxN of SEQ ID NO: 5, LoxP of SEQ ID NO: 12 and Lox2272 of SEQ ID NO: 68.

According to a particular method of realization, the present invention concerns a mutant strain of Sarcocystidae in which said enzyme allowing specific recombination is Cre-recombinase, and said site of specific recombination of an enzyme allowing locus-specific recombination of said deleted rop16I gene, is a site of specific recombination of Cre-recombinase chosen from the following sites: LoxN of SEQ ID NO: 5, LoxP of SEQ ID NO: 12 and Lox2272 of SEQ ID NO: 68,

this specific recombination site being different from the specific recombination sites of Cre-recombinase at the locus of the deleted mic1 gene and at the locus of the deleted mic3 gene.

According to a particular method of realization, the present invention concerns a mutant strain of Toxoplasma spp. in which both genes mic 1 and mic 3, and the gene rop16I are deleted, and which contains at the locus of each of these deleted genes a specific recombination site of the Cre-recombinase, such as

the specific recombination site of Cre-recombinase at the locus of the deleted mic1 gene (called site A) is different from the specific recombination site at the locus of the deleted mic3 gene (called site B),

and the specific recombination site of Cre-recombinase at the locus of the deleted rop16I gene (called site D) is different from the specific recombination site of Cre-recombinase at the locus of the deleted mic1 gene (called site A) and the specific recombination site of

Cre-recombinase at the locus of the deleted mic3 gene (called site B), such as
said site A corresponds to a LoxN site of SEQ ID NO: 5
said site B corresponds to a LoxP site of SEQ ID NO: 12
said site D corresponds to a Lox2272 site of SEQ ID NO: 68;

or such as
said site A corresponds to a LoxP site of SEQ ID NO: 12
said site B corresponds to a LoxN site of SEQ ID NO: 5
said site D corresponds to a Lox2272 site of SEQ ID NO: 68.

Thus, according to a particular method of realization, the present invention concerns a mutant strain of Sarcocystidae in which the said mutant strain is a mutant strain of Toxoplasma spp, in which the two genes mic 1 and mic 3 are deleted, and containing two specific recombination sites of the Cre-recombinase, each of the two sites being respectively at the locus of each of the said deleted genes, the specific recombination site of the Cre-recombinase, at the locus of the deleted mic1 gene being different from that at the locus of the deleted mic3 gene.

wherein the rop16I gene is deleted and said strain contains a specific recombination site of the Cre-recombinase at the locus of said deleted rop16I gene,

said strain containing three specific recombination sites which are respectively:
site A corresponding to the specific recombination site of Cre-recombinase at the locus of the deleted mic1 gene
site B corresponding to the specific recombination site of Cre-recombinase at the locus of the deleted mic3 gene, and
the site D corresponding to the specific recombination site of the Cre-recombinase at the locus of said deleted rop16I gene

such as
said site A is the LoxN site of SEQ ID NO: 5
said site B is the LoxP site of SEQ ID NO: 12,
said site D is the Lox2272 site of SEQ ID NO: 68;

or such as
said site A is the LoxP site of SEQ ID NO: 12
said site B is the LoxN site of SEQ ID NO: 5,
said site D is the Lox2272 site of SEQ ID NO: 68.

According to a particular mode of realization, the present invention concerns a mutant strain, in which the said mutant strain is a mutant strain of Toxoplasma spp,

and wherein the rop16I gene is deleted and contains an enzyme-specific recombination site allowing locus-specific recombination of said deleted rop16I gene, said site being different from said specific recombination site located at the locus of the deleted mic1 gene and said specific recombination site located at the locus of the deleted mic3 gene

and said mutant strain comprising a gene encoding the protein GRA15II, as well as the means necessary for the expression of said protein at the locus of said deleted rop16I gene,

said gene encoding the protein GRA15II at the locus of said deleted rop16I gene, being flanked upstream by said enzyme-specific recombination site allowing locus-specific recombination of said deleted rop16I gene,

in particular in which said enzyme allowing specific recombination is Cre-recombinase, and said specific recombination site of an enzyme allowing locus-specific recombination of said deleted rop16I gene is a specific recombination site of Cre-recombinase selected from the following sites: LoxN of SEQ ID NO: 5, LoxP of SEQ ID NO: 12 and Lox2272 of SEQ ID NO: 68,

this specific recombination site being different from the specific recombination sites of Cre-recombinase at the locus of the deleted mic1 gene and at the locus of the deleted mic3 gene, said strain containing three specific recombination sites which are respectively:
site A corresponding to the specific recombination site of Cre-recombinase at the locus of the deleted mic1 gene
site B corresponding to the specific recombination site of Cre-recombinase at the locus of the deleted mic3 gene, and
the site D corresponding to the specific recombination site of the Cre-recombinase at the locus of said deleted rop16I gene

in particular such as
said site A is the LoxN site,
said site B is site LoxP,
said site D is site Lox2272.

According to a particular method of realization, the present invention concerns a mutant strain of Toxoplasma spp. in which both genes mic 1 and mic 3, and the gene rop16I are deleted, and which contains at the locus of each of these deleted genes a specific recombination site of the Cre-recombinase, such as

the specific recombination site of Cre-recombinase at the locus of the deleted mic1 gene (called site A) is different from the specific recombination site at the locus of the deleted mic3 gene (called site B),

and the specific recombination site of Cre-recombinase at the locus of the deleted rop16I gene (called site D) is different from the specific recombination site of Cre-recombinase at the locus of the deleted mic1 gene (called site A) and the specific recombination site of Cre-recombinase at the locus of the deleted mic3 gene (called site B),

said strain optionally containing a coder for the protein GRA15II as well as the means of expression necessary for the expression of said protein,

and said strain containing DNA heterologous at the locus of the deleted mic1 gene or the deleted mic3 gene or the deleted rop16I gene, different from the heterologous DNA corresponding to the specific recombination sites of the Cre-recombinase, at the respective locus of each of the deleted mic1, mic3 and rop16I genes, so that:
when said heterologous DNA is inserted at the locus of the deleted mic1 gene, it is flanked:
   a first Cre-recombinase-specific recombination site, corresponding to a Cre-recombinase-specific recombination site, at the locus of the deleted mic1 gene (site A), and
   a second specific recombination site (site C), identical to the first specific recombination site located at the locus of the deleted mic1 gene (site A),
when said heterologous DNA is inserted at the locus of the deleted mic3 gene, it is flanked:
   a first Cre-recombinase-specific recombination site, corresponding to a Cre-recombinase-specific recombination site, at the locus of the deleted mic3 gene (site B), and
   a second specific recombination site (site C), identical to the first specific recombination site located at the mic3 gene (site B) deleted,
when said heterologous DNA is inserted at the locus of the deleted rop16I gene, it is flanked:
   a first Cre-recombinase-specific recombination site, corresponding to a Cre-recombinase-specific recombination site, at the locus of the deleted rop16I gene (site D), and
   a second specific recombination site (site C), identical to the first specific recombination site located at the locus of the deleted rop16I gene (site D), it being understood that said strain comprises the elements necessary for the transcription of said heterologous DNA, or the means necessary for the expression of said heterologous DNA when said heterologous DNA encodes at least one protein.

In this particular mode of realization, said mutant strain then contains four specific recombination sites of cre-recombinase defined as follows:
the site A corresponding to said specific recombination site of Cre-recombinase at the locus of the deleted mic1 gene
the site B corresponding to said specific recombination site of Cre-recombinase at the locus of the deleted mic3 gene, and
site C corresponding to a Cre-recombinase specific recombination site at the locus of the deleted mic1 gene (site A) when heterologous DNA is inserted at the locus of the deleted mic1 gene or corresponding to a Cre-recombinase specific recombination site at the locus of the deleted mic3 gene (site B) when heterologous DNA is inserted at the locus of the deleted mic3 gene, or corresponding to a specific recombination site of Cre-recombinase at the locus of the deleted rop16I gene (site D) when heterologous DNA is inserted at the locus of the deleted rop16I gene
the site D corresponding to said specific recombination site of Cre-recombinase at the locus of said deleted rop16I gene

such as, when heterologous DNA is inserted at the locus of the deleted mic1 gene,
said site A corresponds to a LoxN site of SEQ ID NO: 5
said site B corresponds to a LoxP site of SEQ ID NO: 12
said site C corresponds to a LoxN site of SEQ ID NO: 5
said site D corresponds to a Lox2272 site of SEQ ID NO: 68;

or
said site A corresponds to a LoxP site of SEQ ID NO: 12
said site B corresponds to a LoxN site of SEQ ID NO: 5
said site C corresponds to a LoxP site of SEQ ID NO: 12
said site D corresponds to a Lox2272 site of SEQ ID NO: 68;

or such as when heterologous DNA is inserted at the locus of the deleted mic3 gene,
said site A corresponds to a LoxN site of SEQ ID NO: 5
said site B corresponds to a LoxP site of SEQ ID NO: 12
said site C corresponds to a LoxP site of SEQ ID NO: 12
said site D corresponds to a Lox2272 site of SEQ ID NO: 68;

or
said site A corresponds to a LoxP site of SEQ ID NO: 12
said site B corresponds to a LoxN site of SEQ ID NO: 5
said site C corresponds to a LoxN site of SEQ ID NO: 5
said site D corresponds to a Lox2272 site of SEQ ID NO: 68;

or such as when heterologous DNA is inserted at the locus of the deleted rop16I gene,
said site A corresponds to a LoxN site of SEQ ID NO: 5
said site B corresponds to a LoxP site of SEQ ID NO: 12
said site C corresponds to a Lox2272 site of SEQ ID NO: 68
said site D corresponds to a Lox2272 site of SEQ ID NO: 68;

or
said site A corresponds to a LoxP site of SEQ ID NO: 12
said site B corresponds to a LoxN site of SEQ ID NO: 5
said site C corresponds to a Lox2272, SEQ ID NO: 68
said site D corresponds to a Lox2272 site of SEQ ID NO: 68.

According to a particular method of implementation, the present invention concerns a mutant strain of Sarcocystidae in which the said mutant strain is a mutant strain of Toxoplasma spp. which enzyme allows a specific recombination is Cre-recombinase,

wherein the rop16I gene is deleted and said strain contains a specific recombination site of the Cre-recombinase at the locus of said deleted rop16I gene,

said strain containing four specific recombination sites which are respectively:
site A corresponding to the specific recombination site of Cre-recombinase at the locus of the deleted mic1 gene
site B corresponding to the specific recombination site of Cre-recombinase at the locus of the deleted mic3 gene, and
the site C corresponding to said second specific recombination site which flanks said heterologous DNA identical to said first specific recombination site, said first Cre-recombinase specific recombination site corresponding to said Cre-recombinase specific recombination site at the locus of the deleted mic1 gene (site A) or said Cre-recombinase specific recombination site at the locus of the deleted mic3 gene (site B), or to the Cre-recombinase specific recombination site at the locus of the deleted rop16I gene (site D)
the site D corresponding to the specific recombination site of the Cre-recombinase at the locus of said deleted rop16I gene

such as
said site A is the LoxN site of SEQ ID NO: 5
said site B is the LoxP site of SEQ ID NO: 12,
said site C is the LoxN site of SEQ ID NO: 5, said first specific recombination site of Cre-recombinase being site A, and
said site D is the Lox2272 site of SEQ ID NO: 68;

or such as
said site A is the LoxN site of SEQ ID NO: 5
said site B is the LoxP site of SEQ ID NO: 12,
said site C is the LoxP site of SEQ ID NO: 12, said first specific recombination site of Cre-recombinase being site B, and
said site D is the Lox2272 site of SEQ ID NO: 68;

or such as
said site A is the LoxP site of SEQ ID NO: 12
said site B is the LoxN site of SEQ ID NO: 5,
said site C is the LoxP site of SEQ ID NO: 12, said first specific recombination site of Cre-recombinase being site A, and
said site D is the Lox2272 site of SEQ ID NO: 68;

or such as
said site A is the LoxP site of SEQ ID NO: 12
said site B is the LoxN site of SEQ ID NO: 5,
said site C is the LoxN site of SEQ ID NO: 5, said first specific recombination site of Cre-recombinase being site B, and
said site D is the Lox2272 site of SEQ ID NO: 68;

or such as
said site A is the LoxP site of SEQ ID NO: 12
said site B is the LoxN site of SEQ ID NO: 5,
said site C is Lox2272 site SEQ ID NO: 68, said first specific recombination site of Cre-recombinase being site D, and
said site D is the Lox2272 site of SEQ ID NO: 68;

or such as
said site A is the LoxN site of SEQ ID NO: 5
said site B is the LoxP site of SEQ ID NO: 12,
said site C is Lox2272 site SEQ ID NO: 68, said first specific recombination site of Cre-recombinase being site D, and
said site D is the Lox2272 site of SEQ ID NO: 68.

According to a particular mode of realization, the present invention concerns a mutant strain of Sarcocystidae comprising a heterologous DNA as defined above, in which said heterologous DNA encodes a protein of interest.

According to a particular mode of realization, said heterologous DNA cited in any of the modes of realization previously described, is chosen from:

the sequence SEQ ID NO: 212 which codes for the protein SEQ ID NO: 208, the sequence SEQ ID NO: 213 which codes for the protein SEQ ID NO: 209, the sequence SEQ ID NO: 214 which codes for the protein SEQ ID NO: 210, the sequence SEQ ID NO: 215 which codes for the protein SEQ ID NO: 211, the sequence SEQ ID NO: 173 which codes for the protein SEQ ID NO: 167, or the sequence SEQ ID NO: 168 which codes for the protein SEQ ID NO: 165.

According to a particular mode of realization, said heterologous DNA cited in any of the modes of realization previously described, is chosen from:

a sequence encoding the protein SEQ ID NO: 208, a sequence encoding the protein SEQ ID NO: 209, a sequence encoding the protein SEQ ID NO: 210, a sequence encoding the protein SEQ ID NO: 211, a sequence encoding the protein SEQ ID NO: 167, or a sequence encoding the protein SEQ ID NO: 165, depending on the degeneration of the genetic code.

According to a particular mode of realization, the present invention concerns a mutant strain of Sarcocystidae comprising a heterologous DNA as defined above, wherein said protein of interest is an immunogenic heterologous antigen.

Immunogenic heterologous antigen” means any peptide or protein derived from an organism different from said mutant strain and capable of inducing an immune response.

An antigen can correspond to one or more epitopes.

According to a particular mode of realization, the present invention concerns a mutant strain of Sarcocystidae in which the said heterologous DNA encodes at least two proteins of interest and includes the means necessary for their expression, each of the said at least two proteins of interest being translated independently, i.e. they are each controlled by elements necessary for their independent translation.

According to a particular mode of realization, the present invention concerns a mutant strain of Sarcocystidae in which the said heterologous DNA encodes at least two proteins of interest and includes the means necessary for their expression, each of the said at least two proteins of interest being independently translated, and wherein said at least two proteins of interest are immunogenic heterologous antigens.

According to a particular mode of realization, the present invention concerns a mutant strain of Sarcocystidae comprising a heterologous DNA in which said heterologous DNA encodes at least one protein of interest and a resistance protein and the means necessary for the expression of said proteins, each of said proteins of interest and resistance being independently translated.

According to a particular mode of realization, the present invention concerns a mutant strain of Sarcocystidae comprising a heterologous DNA in which said heterologous DNA encodes at least one protein of interest and a resistance protein and the means necessary for expression of said proteins, each of said proteins of interest and resistance being independently translated, and in which said at least one protein of interest is an immunogenic heterologous antigen.

According to a particular mode of implementation, the present invention concerns a mutant strain of Sarcocystidae comprising a heterologous DNA encoding an immunogenic heterologous antigen as defined above, wherein said immunogenic heterologous antigen is an immunogenic heterologous virus antigen.

According to a particular method of implementation, the present invention concerns a mutant strain of Sarcocystidae comprising a heterologous DNA encoding an immunogenic heterologous antigen as defined above in which said immunogenic heterologous antigen is an immunogenic heterologous antigen of the Influenza virus.

According to a particular method of implementation, the present invention concerns a mutant strain of Sarcocystidae as defined above, comprising a heterologous DNA encoding an immunogenic heterologous antigen, wherein said immunogenic heterologous antigen is an immunogenic heterologous antigen of the Influenza virus selected from: the protein of SEQ ID NO: 208 (N-ter fragment of a human influenza virus I), or the protein of SEQ ID NO: 209 (N-ter fragment of the M2 protein of a swine influenza virus), or the protein of SEQ ID NO: 201 (N-ter fragment of the M2 protein of an avian influenza virusI) or the protein of SEQ ID NO: 211 (N-ter fragment of the M2 protein of an avian influenza virusII) or the protein of SEQ ID NO: 167, corresponding to the fusion, in order, of two proteins SEQ ID NO: 208, a protein SEQ ID NO: 209, a protein SEQ ID NO: 210 and a protein SEQ ID NO: 211, each spaced by a linker to allow a good conformation of the fusion protein SEQ ID NO: 167,

or the protein SEQ ID NO: 165, corresponding to the fusion, in order, of the SAG1 protein of T. gondii SEQ ID NO: 166, two proteins SEQ ID NO: 208, a protein SEQ ID NO: 209, a protein SEQ ID NO: 210 and a protein SEQ ID NO: 211.

According to another particular mode of realization, the two proteins SEQ ID NO: 208, the protein SEQ ID NO: 209, the protein SEQ ID NO: 210 and the protein SEQ ID NO: 211, can be fused in one of the following orders, always ensuring that they are spaced by a linker:

[‘SEQ ID NO: 211’, ‘SEQ ID NO: 208’, ‘SEQ ID NO: 210’, ‘SEQ ID NO: 208’, ‘SEQ ID NO: 209’]

[‘SEQ ID NO: 210’, ‘SEQ ID NO: 211’, ‘SEQ ID NO: 209’, ‘SEQ ID NO: 208’, ‘SEQ ID NO: 208’]

[‘SEQ ID NO: 210’, ‘SEQ ID NO: 208’, ‘SEQ ID NO: 211’, ‘SEQ ID NO: 208’, ‘SEQ ID NO: 209’]

[‘SEQ ID NO: 208’, ‘SEQ ID NO: 208’, ‘SEQ ID NO: 209’, ‘SEQ ID NO: 210’, ‘SEQ ID NO: 211’]

[‘SEQ ID NO: 210’, ‘SEQ ID NO: 209’, ‘SEQ ID NO: 208’, ‘SEQ ID NO: 211’, ‘SEQ ID NO: 208’]

[‘SEQ ID NO: 210’, ‘SEQ ID NO: 208’, ‘SEQ ID NO: 208’, ‘SEQ ID NO: 209’, ‘SEQ ID NO: 211’]

[‘SEQ ID NO: 210’, ‘SEQ ID NO: 211’, ‘SEQ ID NO: 208’, ‘SEQ ID NO: 209’, ‘SEQ ID NO: 208’]

[‘SEQ ID NO: 208’, ‘SEQ ID NO: 208’, ‘SEQ ID NO: 211’, ‘SEQ ID NO: 209’, ‘SEQ ID NO: 210’]

[‘SEQ ID NO: 210’, ‘SEQ ID NO: 208’, ‘SEQ ID NO: 211’, ‘SEQ ID NO: 209’, ‘SEQ ID NO: 208’]

[‘SEQ ID NO: 209’, ‘SEQ ID NO: 208’, ‘SEQ ID NO: 210’, ‘SEQ ID NO: 208’, ‘SEQ ID NO: 211’]

[‘SEQ ID NO: 210’, ‘SEQ ID NO: 208’, ‘SEQ ID NO: 208’, ‘SEQ ID NO: 211’, ‘SEQ ID NO: 209’]

[‘SEQ ID NO: 208’, ‘SEQ ID NO: 210’, ‘SEQ ID NO: 208’, ‘SEQ ID NO: 209’, ‘SEQ ID NO: 211’]

[‘SEQ ID NO: 209’, ‘SEQ ID NO: 211’, ‘SEQ ID NO: 208’, ‘SEQ ID NO: 208’, ‘SEQ ID NO: 210’]

[‘SEQ ID NO: 208’, ‘SEQ ID NO: 208’, ‘SEQ ID NO: 211’, ‘SEQ ID NO: 210’, ‘SEQ ID NO: 209’]

[‘SEQ ID NO: 209’, ‘SEQ ID NO: 211’, ‘SEQ ID NO: 208’, ‘SEQ ID NO: 210’, ‘SEQ ID NO: 208’]

[‘SEQ ID NO: 209’, ‘SEQ ID NO: 208’, ‘SEQ ID NO: 211’, ‘SEQ ID NO: 210’, ‘SEQ ID NO: 208’]

[‘SEQ ID NO: 208’, ‘SEQ ID NO: 209’, ‘SEQ ID NO: 210’, ‘SEQ ID NO: 211’, ‘SEQ ID NO: 208’]

[‘SEQ ID NO: 209’, ‘SEQ ID NO: 210’, ‘SEQ ID NO: 208’, ‘SEQ ID NO: 211’, ‘SEQ ID NO: 208’]

[‘SEQ ID NO: 211’, ‘SEQ ID NO: 208’, ‘SEQ ID NO: 210’, ‘SEQ ID NO: 209’, ‘SEQ ID NO: 208’]

[‘SEQ ID NO: 210’, ‘SEQ ID NO: 208’, ‘SEQ ID NO: 209’, ‘SEQ ID NO: 211’, ‘SEQ ID NO: 208’]

[‘SEQ ID NO: 211’, ‘SEQ ID NO: 209’, ‘SEQ ID NO: 210’, ‘SEQ ID NO: 208’, ‘SEQ ID NO: 208’]

[‘SEQ ID NO: 208’, ‘SEQ ID NO: 211’, ‘SEQ ID NO: 210’, ‘SEQ ID NO: 209’, ‘SEQ ID NO: 208’]

[‘SEQ ID NO: 208’, ‘SEQ ID NO: 210’, ‘SEQ ID NO: 208’, ‘SEQ ID NO: 211’, ‘SEQ ID NO: 209’]

[‘SEQ ID NO: 209’, ‘SEQ ID NO: 208’, ‘SEQ ID NO: 211’, ‘SEQ ID NO: 208’, ‘SEQ ID NO: 210’]

[‘SEQ ID NO: 210’, ‘SEQ ID NO: 209’, ‘SEQ ID NO: 211’, ‘SEQ ID NO: 208’, ‘SEQ ID NO: 208’]

[‘SEQ ID NO: 209’, ‘SEQ ID NO: 208’, ‘SEQ ID NO: 208’, ‘SEQ ID NO: 210’, ‘SEQ ID NO: 211’]

[‘SEQ ID NO: 208’, ‘SEQ ID NO: 211’, ‘SEQ ID NO: 208’, ‘SEQ ID NO: 209’, ‘SEQ ID NO: 210’]

[‘SEQ ID NO: 209’, ‘SEQ ID NO: 210’, ‘SEQ ID NO: 208’, ‘SEQ ID NO: 208’, ‘SEQ ID NO: 211’]

[‘SEQ ID NO: 209’, ‘SEQ ID NO: 208’, ‘SEQ ID NO: 210’, ‘SEQ ID NO: 211’, ‘SEQ ID NO: 208’]

[‘SEQ ID NO: 211’, ‘SEQ ID NO: 210’, ‘SEQ ID NO: 209’, ‘SEQ ID NO: 208’, ‘SEQ ID NO: 208’]

[‘SEQ ID NO: 208’, ‘SEQ ID NO: 208’, ‘SEQ ID NO: 209’, ‘SEQ ID NO: 211’, ‘SEQ ID NO: 210’]

[‘SEQ ID NO: 208’, ‘SEQ ID NO: 211’, ‘SEQ ID NO: 209’, ‘SEQ ID NO: 210’, ‘SEQ ID NO: 208’]

[‘SEQ ID NO: 208’, ‘SEQ ID NO: 211’, ‘SEQ ID NO: 209’, ‘SEQ ID NO: 208’, ‘SEQ ID NO: 210’]

[‘SEQ ID NO: 209’, ‘SEQ ID NO: 210’, ‘SEQ ID NO: 211’, ‘SEQ ID NO: 208’, ‘SEQ ID NO: 208’]

[‘SEQ ID NO: 208’, ‘SEQ ID NO: 208’, ‘SEQ ID NO: 210’, ‘SEQ ID NO: 209’, ‘SEQ ID NO: 211’]

[‘SEQ ID NO: 208’, ‘SEQ ID NO: 209’, ‘SEQ ID NO: 211’, ‘SEQ ID NO: 208’, ‘SEQ ID NO: 210’]

[‘SEQ ID NO: 211’, ‘SEQ ID NO: 209’, ‘SEQ ID NO: 208’, ‘SEQ ID NO: 208’, ‘SEQ ID NO: 210’]

[‘SEQ ID NO: 210’, ‘SEQ ID NO: 211’, ‘SEQ ID NO: 208’, ‘SEQ ID NO: 208’, ‘SEQ ID NO: 209’]

[‘SEQ ID NO: 208’, ‘SEQ ID NO: 211’, ‘SEQ ID NO: 208’, ‘SEQ ID NO: 210’, ‘SEQ ID NO: 209’]

[‘SEQ ID NO: 211’, ‘SEQ ID NO: 210’, ‘SEQ ID NO: 208’, ‘SEQ ID NO: 209’, ‘SEQ ID NO: 208’]

[‘SEQ ID NO: 208’, ‘SEQ ID NO: 210’, ‘SEQ ID NO: 211’, ‘SEQ ID NO: 208’, ‘SEQ ID NO: 209’]

[‘SEQ ID NO: 208’, ‘SEQ ID NO: 210’, ‘SEQ ID NO: 209’, ‘SEQ ID NO: 211’, ‘SEQ ID NO: 208’]

[‘SEQ ID NO: 211’, ‘SEQ ID NO: 208’, ‘SEQ ID NO: 208’, ‘SEQ ID NO: 209’, ‘SEQ ID NO: 210’]

[‘SEQ ID NO: 208’, ‘SEQ ID NO: 209’, ‘SEQ ID NO: 208’, ‘SEQ ID NO: 210’, ‘SEQ ID NO: 211’]

[‘SEQ ID NO: 208’, ‘SEQ ID NO: 208’, ‘SEQ ID NO: 210’, ‘SEQ ID NO: 211’, ‘SEQ ID NO: 209’]

[‘SEQ ID NO: 210’, ‘SEQ ID NO: 209’, ‘SEQ ID NO: 208’, ‘SEQ ID NO: 208’, ‘SEQ ID NO: 211’]

[‘SEQ ID NO: 209’, ‘SEQ ID NO: 211’, ‘SEQ ID NO: 210’, ‘SEQ ID NO: 208’, ‘SEQ ID NO: 208’]

[‘SEQ ID NO: 208’, ‘SEQ ID NO: 210’, ‘SEQ ID NO: 211’, ‘SEQ ID NO: 209’, ‘SEQ ID NO: 208’]

[‘SEQ ID NO: 210’, ‘SEQ ID NO: 208’, ‘SEQ ID NO: 209’, ‘SEQ ID NO: 208’, ‘SEQ ID NO: 211’]

[‘SEQ ID NO: 211’, ‘SEQ ID NO: 210’, ‘SEQ ID NO: 208’, ‘SEQ ID NO: 208’, ‘SEQ ID NO: 209’]

[‘SEQ ID NO: 211’, ‘SEQ ID NO: 208’, ‘SEQ ID NO: 209’, ‘SEQ ID NO: 210’, ‘SEQ ID NO: 208’]

[‘SEQ ID NO: 211’, ‘SEQ ID NO: 209’, ‘SEQ ID NO: 208’, ‘SEQ ID NO: 210’, ‘SEQ ID NO: 208’]

[‘SEQ ID NO: 208’, ‘SEQ ID NO: 210’, ‘SEQ ID NO: 209’, ‘SEQ ID NO: 208’, ‘SEQ ID NO: 211’]

[‘SEQ ID NO: 211’, ‘SEQ ID NO: 208’, ‘SEQ ID NO: 208’, ‘SEQ ID NO: 210’, ‘SEQ ID NO: 209’]

[‘SEQ ID NO: 208’, ‘SEQ ID NO: 209’, ‘SEQ ID NO: 208’, ‘SEQ ID NO: 211’, ‘SEQ ID NO: 210’]

[‘SEQ ID NO: 208’, ‘SEQ ID NO: 209’, ‘SEQ ID NO: 210’, ‘SEQ ID NO: 208’, ‘SEQ ID NO: 211’]

[‘SEQ ID NO: 209’, ‘SEQ ID NO: 208’, ‘SEQ ID NO: 208’, ‘SEQ ID NO: 211’, ‘SEQ ID NO: 210’]

[‘SEQ ID NO: 208’, ‘SEQ ID NO: 209’, ‘SEQ ID NO: 211’, ‘SEQ ID NO: 210’, ‘SEQ ID NO: 208’]

[‘SEQ ID NO: 211’, ‘SEQ ID NO: 208’, ‘SEQ ID NO: 209’, ‘SEQ ID NO: 208’, ‘SEQ ID NO: 210’]

[‘SEQ ID NO: 208’, ‘SEQ ID NO: 211’, ‘SEQ ID NO: 210’, ‘SEQ ID NO: 208’, ‘SEQ ID NO: 209’],

the protein resulting from the fusion of these proteins can also be expressed in fusion with the SAG1 protein of T. gondii SEQ ID NO: 166.

According to a particular method of implementation, the present invention concerns a mutant strain of Sarcocystidae as defined above, in which said immunogenic heterologous antigen is an immunogenic heterologous bacterial antigen.

According to a particular method of implementation, the present invention concerns a mutant strain of Sarcocystidae as defined above, in which said immunogenic heterologous antigen is an immunogenic heterologous parasite antigen.

According to a particular method of execution, said heterologous DNA codes for an immunogenic heterologous antigen of viruses, parasites or bacteria, and in particular consists of the nucleotide sequence SEQ ID NO: 173, encoding the antigen of the Influenza virus SEQ ID NO: 167.

According to a particular mode of realization, the present invention concerns a mutant strain of Sarcocystidae as defined above in which said mutant strain is a strain of Toxoplasma gondii and in which the mic1 gene and the mic3 gene are deleted and in which
the specific recombination site of Cre-recombinase at the locus of the deleted mic1 gene is the LoxN site of SEQ ID NO: 5 and
the specific recombination site of Cre-recombinase at the locus of the deleted mic3 gene is the LoxP site of SEQ ID NO: 12.

According to a particular mode of realization, the present invention concerns a mutant strain of Sarcocystidae as defined above in which said mutant strain is a strain of Toxoplasma gondii, in which the mic1 gene and the mic3 gene are deleted and in which
the specific recombination site of Cre-recombinase at the locus of the deleted mic1 gene is the LoxN site of SEQ ID NO: 5 and
the specific recombination site of Cre-recombinase at the locus of the deleted mic3 gene is the LoxP site of SEQ ID NO: 12.

said strain comprising a heterologous DNA SEQ ID NO: 168 allowing the expression of a protein SEQ ID NO: 165,

said heterologous DNA being at the locus of the deleted mic1 gene, and being flanked upstream by a first recombination site corresponding to said specific recombination site of the deleted mic1 gene is the LoxN site of SEQ ID NO: 5 and downstream by a second specific recombination site of the Cre-combinase LoxN of SEQ ID NO: 5.

According to a particular mode of realization, the present invention concerns a mutant strain of Sarcocystidae as defined above in which said mutant strain is a strain of Toxoplasma gondii, in which the mic1 gene and the mic3 gene are deleted and in which
the specific recombination site of Cre-recombinase at the locus of the deleted mic1 gene is the LoxN site of SEQ ID NO: 5 and
the specific recombination site of Cre-recombinase at the locus of the deleted mic3 gene is the LoxP site of SEQ ID NO: 12.

said strain comprising a heterologous DNA SEQ ID NO: 168 allowing the expression of a protein SEQ ID NO: 165,

said heterologous DNA being at the locus of the deleted mic3 gene, and being flanked upstream by a first recombination site corresponding to said specific recombination site of the deleted mic3 gene is the LoxP site of SEQ ID NO: 12 and downstream by a second specific recombination site of the Cre-combinase LoxP of SEQ ID NO: 12.

According to a particular method of realization, the present invention concerns a mutant strain of Sarcocystidae, said mutant strain being a strain of Toxoplasma gondii, in which the mic 1 genes, the mic 3 gene, and the rop16I gene are deleted, and in which
the specific recombination site of Cre-recombinase at the locus of the deleted mic1 gene is the LoxN site of SEQ ID NO: 5, and
the specific recombination site of Cre-recombinase at the locus of the deleted mic3 gene is the LoxP site of SEQ ID NO: 12, and
the specific recombination site of Cre-recombinase at the locus of the deleted rop16I gene is Lox2272 site of SEQ ID NO: 68.

According to a particular mode of realization, the present invention concerns a mutant strain according to the invention, wherein the said mutant strain is a strain of Toxoplasma gondii selected from
a strain in which
   the mic1 gene and the mic3 gene are deleted and wherein the specific recombination site of the Cre-recombinase at the locus of the deleted mic1 gene is the LoxN site of SEQ ID NO: 5 and
   the specific recombination site of Cre-recombinase at the locus of the deleted mic3 gene is the LoxP site of SEQ ID NO: 12.
a strain in which the mic 1 gene, the mic 3 gene and the rop16I gene are deleted, and comprising a gene encoding the protein GRA15II, as well as the means necessary for the expression of said protein, at the locus of said deleted rop16I gene,
said gene encoding the protein GRA15II at the locus of said deleted rop16I gene, being flanked upstream by a recombination site specific for Cre-recombinase, in which
   the specific recombination site of Cre-recombinase at the locus of the deleted mic1 gene is the LoxN site of SEQ ID NO: 5, and
   the specific recombination site of Cre-recombinase at the locus of the deleted mic3 gene is the LoxP site of SEQ ID NO: 12, and
   said Cre-recombinase specific recombination site which upstream flanks said gene encoding the GRA15II protein at the locus of the deleted rop16I gene, is the Lox2272 site of SEQ ID NO: 68.

According to a particular method of implementation, the present invention concerns a mutant strain of Toxoplasma spp. wherein the genes mic1, mic3 and rop16I are deleted, containing three Cre-recombinase specific recombination sites, at the respective locus of each of said deleted genes, the Cre-recombinase specific recombination site, at the locus of the deleted mic1 gene being different from that at the locus of the deleted mic3 gene and the recombination site specific to the locus of the deleted rop16I gene being different from the recombination sites specific to the loci of the deleted mic1 and mic3 genes and said strain containing DNA heterologous to the locus of the deleted mic1 gene or the locus of the deleted mic3 gene or the locus of the deleted rop16I gene, said heterologous DNA being different from heterologous DNA corresponding to the specific recombination sites of the Cre-recombinase, at the respective locus of each of the deleted mic1, mic3 and rop16I genes, in such a way that:
when said heterologous DNA is inserted at the locus of the deleted mic1 gene, it is flanked:
   a first Cre-recombinase-specific recombination site, corresponding to a Cre-recombinase-specific recombination site, at the locus of the deleted mic1 gene (site A), and
   a second Cre-recombinase specific recombination site (site C), identical to the first Cre-recombinase specific recombination site located at the locus of the deleted mic1 gene (site A),
when said heterologous DNA is inserted at the locus of the deleted mic3 gene, it is flanked:
   a first Cre-recombinase-specific recombination site, corresponding to a Cre-recombinase-specific recombination site, at the locus of the deleted mic3 gene (site B), and
   a second Cre-recombinase specific recombination site (site C), identical to the first Cre-recombinase specific recombination site located at the mic3 gene (site B) deleted,
when said heterologous DNA is inserted at the locus of the deleted rop16I gene, it is flanked:
   a first Cre-recombinase-specific recombination site, corresponding to a Cre-recombinase-specific recombination site, at the locus of the deleted rop16I gene (site D), and
   a second Cre-recombinase specific recombination site (site C), identical to the first Cre-recombinase specific recombination site located at the locus of the deleted rop16I gene (site D),

said strain comprising the elements necessary for the transcription of said heterologous DNA, or the means necessary for the expression of said heterologous DNA when said heterologous DNA encodes at least one protein,

said mutant strain then containing four specific recombination sites of Cre-recombinase defined as follows:
the site A corresponding to said specific recombination site of Cre-recombinase at the locus of the deleted mic1 gene
the site B corresponding to said specific recombination site of Cre-recombinase at the locus of the deleted mic3 gene, and
site C corresponding to a Cre-recombinase specific recombination site at the locus of the deleted mic1 gene (site A) when heterologous DNA is inserted at the locus of the deleted mic1 gene or corresponding to a Cre-recombinase specific recombination site at the locus of the deleted mic3 gene (site B) when heterologous DNA is inserted at the locus of the deleted mic3 gene, or corresponding to a specific recombination site of Cre-recombinase at the locus of the deleted rop16I gene (site D) when heterologous DNA is inserted at the locus of the deleted rop16I gene
the site D corresponding to said specific recombination site of Cre-recombinase at the locus of said deleted rop16I gene

such as, when heterologous DNA is inserted at the locus of the deleted mic1 gene,
said site A corresponds to a LoxN site of SEQ ID NO: 5
said site B corresponds to a LoxP site of SEQ ID NO: 12
said site C corresponds to a LoxN site of SEQ ID NO: 5
said site D corresponds to a Lox2272 site of SEQ ID NO: 68;

or such as when heterologous DNA is inserted at the locus of the deleted mic3 gene,
said site A corresponds to a LoxN site of SEQ ID NO: 5
said site B corresponds to a LoxP site of SEQ ID NO: 12
said site C corresponds to a LoxP site of SEQ ID NO: 12
said site D corresponds to a Lox2272 site of SEQ ID NO: 68;

or such as when heterologous DNA is inserted at the locus of the deleted rop16I gene,
said site A corresponds to a LoxN site of SEQ ID NO: 5
said site B corresponds to a LoxP site of SEQ ID NO: 12
said site C corresponds to a Lox2272 site of SEQ ID NO: 68
said site D corresponds to a Lox2272 site of SEQ ID NO: 68.

According to a particular method of implementation, the present invention concerns a mutant strain of Sarcocystidae in which the said mutant strain is a strain of Neospora caninum and in which the mic1 gene and the mic3 gene are deleted and in which
the specific recombination site of Cre-recombinase at the locus of the deleted mic1 gene is the LoxP site of SEQ ID NO: 12 and
the specific recombination site of Cre-recombinase at the locus of the deleted mic3 gene is the LoxN site of SEQ ID NO: 5.

According to a particular mode of realization, the present invention concerns a mutant strain of Sarcocystidae as defined above in which said mutant strain is a strain of Neospora caninum, in which the mic1 gene and the mic3 gene are deleted and in which
the specific recombination site of Cre-recombinase at the locus of the deleted mic1 gene is the LoxP site of SEQ ID NO: 12.
the specific recombination site of Cre-recombinase at the locus of the deleted mic3 gene is the LoxN site of SEQ ID NO: 5 and

said strain comprising a heterologous DNA SEQ ID NO: 168 allowing the expression of a protein SEQ ID NO: 165,

said heterologous DNA being at the locus of the deleted mic1 gene, and being flanked upstream by a first recombination site corresponding to said specific recombination site of the deleted mic1 gene is the LoxP site of SEQ ID NO: 12 and downstream by a second specific recombination site of the Cre-combinase LoxP of SEQ ID NO: 12.

According to a particular mode of realization, the present invention concerns a mutant strain of Sarcocystidae as defined above in which said mutant strain is a strain of Neospora caninum, in which the mic1 gene and the mic3 gene are deleted and in which
the specific recombination site of Cre-recombinase at the locus of the deleted mic1 gene is the LoxP site of SEQ ID NO: 12.
the specific recombination site of Cre-recombinase at the locus of the deleted mic3 gene is the LoxN site of SEQ ID NO: 5 and

said strain comprising a heterologous DNA SEQ ID NO: 168 allowing the expression of a protein SEQ ID NO: 165,

said heterologous DNA being at the locus of the deleted mic3 gene, and being flanked upstream by a first recombination site corresponding to said specific recombination site of the deleted mic3 gene is the LoxN site of SEQ ID NO: 5 and downstream by a second specific recombination site of the LoxN Cre-recombinase of SEQ ID NO: 5.

This invention also concerns the use of a mutant strain as defined above, not containing heterologous DNA different from heterologous DNA corresponding to the specific recombination sites of Cre-recombinase at the respective locus of each of the said deleted genes, for the targeted insertion of heterologous DNA, with the exception of use for therapeutic purposes.

This invention also concerns a mutant strain containing heterologous DNA encoding an immunogenic heterologous antigen as defined above, for use as a medicinal product, in particular as a vaccine or as an immunostimulant.

According to a particular method of implementation, this invention also concerns a mutant strain containing a heterologous DNA encoding an immunogenic heterologous antigen as defined above, for its use in the prevention of infectious diseases of viral, parasitic or bacterial origin.

According to a particular method of implementation, this invention also concerns a mutant strain containing a heterologous DNA encoding an immunogenic heterologous antigen as defined above, for its use in the prevention of infectious diseases of viral, parasitic or bacterial origin in a mammal or birds.

According to a particular method of implementation, the present invention also concerns a mutant strain containing a heterologous DNA encoding an immunogenic heterologous antigen as defined above, for its use in the prevention of infectious diseases of viral, parasitic or bacterial origin in a mammal, the said mammal being in particular chosen from humans, ovidae, goats, pigs, cattle, equidae, camelids, canidae or felids.

According to a particular method of implementation, this invention also concerns a mutant strain containing a heterologous DNA encoding an immunogenic heterologous antigen as defined above, for its use in the prevention of infectious diseases of viral, parasitic or bacterial origin in a bird, the said bird being in particular chosen from hens, turkeys, guinea fowls, ducks, geese, quails, pigeons, pheasants, partridges, ostriches, rheas, emus and kiwifruit bred or kept in captivity for breeding, the production of meat or eggs for consumption or for the supply of restocking game.

According to a particular method of implementation, this invention also concerns a mutant strain containing a heterologous DNA encoding an immunogenic heterologous antigen as defined above, for its use in the prevention of an infectious disease affecting the digestive system, causing diarrhoea, affecting food digestibility or intestinal absorption.

According to a particular method of implementation, this invention also concerns a mutant strain containing a heterologous DNA encoding an immunogenic heterologous antigen as defined above, for its use in the prevention of an infectious pathology affecting the central or peripheral nervous system and all consequences on other systems associated therewith.

According to a particular method of implementation, this invention also concerns a mutant strain containing a heterologous DNA encoding an immunogenic heterologous antigen as defined above, for its use in the prevention of an infectious pathology affecting the musculoskeletal or articular system, in particular diseases of infectious origin causing myositis, arthritis or theft.

According to a particular method of implementation, this invention also concerns a mutant strain containing a heterologous DNA encoding an immunogenic heterologous antigen as defined above, its use in the prevention of an infectious pathology affecting the respiratory system and in particular catarrhal fevers, obstructive abscesses, perforations and emphysema of infectious origin, tracheitis, bronchitis, pneumonia, pleurisy or in the particular case of birds, aerosacculitis.

According to a particular method of implementation, this invention also concerns a mutant strain containing a heterologous DNA encoding an immunogenic heterologous antigen as defined above, for its use in the prevention of an infectious pathology affecting the reproductive system, fertility and fertility, in particular, an infectious pathology affecting the normal development of the male or female reproductive system, the physiology of the reproductive cycle in females, affecting the proper development of pregnancy in mammals including foetal development or affecting the quality of eggs, in particular, infectious pathologies inducing metritis, salpingitis, mastitis or egg fall syndrome.

According to a particular method of implementation, this invention also concerns a mutant strain containing a heterologous DNA encoding an immunogenic heterologous antigen as defined above, for its use in the prevention of a pathology of infectious origin affecting the cardiovascular system, bone marrow or blood, in particular in the prevention of diseases of infectious origin causing alteration in the genesis of blood figured elements and sepsis including associated consequences on other organs.

According to a particular method of implementation, this invention also concerns a mutant strain containing heterologous DNA encoding an immunogenic heterologous antigen as defined above, for use in preventing the carrying of viruses, bacteria or parasites by an animal or by-product thereof and having a potentially zoonotic character, in particular, for use in the prevention of carrying in Salmonella spp. in animals, zoonotic Escherichia spp., Clostridia, mycobacteria including tuberculosis.

According to a particular method of implementation, this invention also concerns a mutant strain containing a heterologous DNA encoding an immunogenic heterologous antigen as defined above, for its use in the prevention of viral infectious pathology caused by a virus belonging to the following groups:
Group I, II of the Baltimore Classification comprising viruses whose genome consists of DNA capable of infecting a mammal or bird, in particular a natural virus belonging to the Herpesviridae family, and more particularly the Epstein-Barr virus responsible for human mononucleosis, bovine herpes virus type I responsible for infectious bovine rhinotracheitis or the Marek disease virus in domestic poultry.
Group III, IV and V of the Baltimore Classification including viruses whose genome consists of single or double-stranded RNA and which are likely to affect a mammal or bird, in particular viruses belonging to the family Orthomyxoviridae and more particularly human, avian and porcine influenza viruses.
Group VI of the Baltimore Classification including RNA viruses requiring DNA retrotranscription for viral multiplication and capable of infecting a mammal or bird, in particular Retroviridae family viruses, such as human immunodeficiency virus (HIV), feline immunodeficiency virus (FIV), arthritis and caprine encephalitis virus or feline leucosis virus FELV.
Group VII of the Baltimore Classification including DNA viruses requiring RNA retrotranscription for viral multiplication and likely to infect a mammal or bird, in particular viruses of the Hepadnaviridae family, and more particularly human hepatitis B virus or beech duck hepatitis virus (DHBV).

According to a particular method of implementation, this invention concerns a mutant strain containing heterologous DNA encoding a heterologous antigen SEQ ID NO: 165, for use in the prevention of influenza caused by the influenza virus Influenza.

According to a particular method of implementation, this invention also concerns a mutant strain containing a heterologous DNA encoding an immunogenic heterologous antigen as defined above, for its use in the prevention of an infectious disease of bacterial origin (or caused by a toxin from a bacterium), said bacterium may belong to the following groups:
GRAM positive shells that are likely to affect a mammal or bird directly or through one of these by-products. In particular bacteria belonging to the genus Staphylococcus, Streptococcus or Enteroccoccus;
GRAM negative shells which are likely to affect directly or through one of these by-products, a mammal or a bird, in particular bacteria belonging to the genus Neisseria;
Positive GRAM bacilli that are likely to affect directly or through any of these by-products, a mammal or a bird, in particular bacteria belonging to the genera Listeria, Erysipelothryx, Corynebacterium or Bacillus;
Negative GRAM bacilli that are likely to affect directly or through one of these by-products, a mammal or a bird, in particular bacteria belonging to the Enterobacteriacae family. Pseudomonaceae, Vibrionaceae or belonging to the genera Escherichia, Brucella, Haemophilus, Pasteurella, Bordetella, Legionella, Ornithobacterium or Salmonella;
Bacteria in spiral form GRAM positive or negative which are likely to affect directly or through one of these by-products, a mammal or a bird, in particular bacteria belonging to the genus Treponema, Leptospira or Borrelia;
Bacteria of the mycoplasmic type which are likely to affect directly or through one of these by-products, a mammal or a bird, in particular bacteria belonging to the genera Mycoplasma and Ureaplasma;
Unsurprising bacteria with the ability to invade a mammal or bird cell, particularly bacteria belonging to the genera Chlamydia, Rickettsia or Micobacterium.

According to a particular method of implementation, this invention also concerns a mutant strain containing a heterologous DNA encoding an immunogenic heterologous antigen as defined above, for its use in the prevention of a pathology originating from a parasitic infection, the parasite may belong to the following groups:
Protozoa likely to directly affect a mammal or a bird, in particular protozoa belonging to the sporozoan, Rhizoflagellate, ciliate or genera Encephalitozoon, Enterocytozoon or blastocystis branch;
Nemathelminths likely to directly affect a mammal or a bird, in particular Nemathelminths belonging to the genus Trichuris, Ascaris, Anisakis, Necator, Strongyloids, Toxocara, Ancylostoma, Trichinella, Loa, Onchocerca, Wichereria, or Enterobius;
Plathelminths likely to directly affect a mammal or a bird, in particular Plathelminths belonging to the genera Fasciola, Paragonimus, Opisthorchis, Sasciolopsis, Dicrocoelium, Clonorchis, Taenia, Diphyllobothrium, Echinococcus, Multiceps, Hymenolepsis and Shistosoma;
Arthropods likely to be responsible for a disease or the vectorization of an infectious agent towards a mammal or a bird, in particular arthropods belonging to the order Anoplurae of heteroptera, Shiphonaptera, Diptera, Brachycera or Acarus.

According to a particular method of implementation, this invention also concerns a mutant strain containing a heterologous DNA encoding an immunogenic heterologous antigen as defined above, for its use as an immunostimulant.

“Immunostimulant” means that said mutant strain, possibly containing heterologous DNA encoding an immunogenic heterologous antigen, stimulates immune defences (such as a vaccine, for example).

This invention also concerns a pharmaceutical composition comprising a mutant strain as defined above and a pharmaceutically acceptable carrier.

According to a particular method of implementation, the present invention also concerns a pharmaceutical composition, comprising at least one product chosen from: an adjuvant, a stabilizer or a preservative, or a mixture thereof.

According to a particular method of implementation, the present invention concerns a pharmaceutical composition, formulated in the form of a unit dose varying from 10² to 10⁹ tachyzoites of a mutant strain as defined above.

This invention also concerns a vaccine composition comprising a mutant strain as defined above and a pharmaceutically acceptable carrier.

This invention also concerns a method for the prevention of an infectious disease of viral, parasitic or bacterial origin, including a step of administration to a mammal or bird of a mutant strain.

This invention also concerns a process for obtaining a mutant strain of Sarcocystidae in which the mic1 and mic3 genes are deleted, from a wild strain, comprising:
a step A comprising or consisting of deletion of the mic3 gene by homologous recombination and insertion of a selection cassette framed by two identical specific recombination sites, followed by excision of said selection cassette by cre-recombinase, following which only one of the two specific recombination sites framing the cassette remains integrated at the locus of said deleted mic3 gene,
a step B comprising or consisting of deletion of the mic1 gene by homologous recombination and insertion of a selection cassette framed by two identical specific recombination sites, followed by excision of said selection cassette by cre-recombinase, following which only one of the two specific recombination sites framing the cassette remains integrated at the locus of said deleted mic1 gene

the steps A and B can be carried out in an order A followed by B or B followed by A, and the two specific recombination sites framing the selection cassette inserted at the mic1 gene being different from the two specific recombination sites framing the selection cassette inserted at the mic3 gene.

This invention also concerns a process for obtaining a mutant strain of Sarcocystidae in which the genes mic1, mic3 and rop16I are deleted and in which a gene encoding the protein GRA15II is integrated into the locus of the deleted rop16I gene, from a wild strain, comprising:
A step A comprising or consisting of deletion of the mic3 gene by homologous recombination and insertion of a selection cassette framed by two identical specific recombination sites, followed by excision of said selection cassette by cre-recombinase, following which only one of the two specific recombination sites framing the cassette remains integrated at the locus of said deleted mic3 gene,
A step B comprising or consisting of deletion of the mic1 gene by homologous recombination and insertion of a selection cassette framed by two identical specific recombination sites, followed by a step of excision of said selection cassette by cre-recombinase, following which only one of the two specific recombination sites framing the cassette remains integrated at the locus of said deleted mic1 gene the two specific recombination sites framing the selection cassette inserted at the mic1 gene being different from the two specific recombination sites framing the selection cassette inserted at the mic3 gene
a step C comprising or consisting of deletion of the rop16I gene by homologous recombination and insertion of a cassette comprising a selection cassette framed by two specific recombination sites, and comprising downstream of this selection cassette a sequence encoding the gra15II gene, followed by a step of excision of said selection cassette by cre-recombinase, following which said sequence encoding the gra15II gene is then integrated at the locus of the rop16I gene deleted downstream of a single specific recombination site

the two specific recombination sites framing the selection cassette inserted at the locus of the rop16I gene being different from the two specific recombination sites framing the selection cassettes inserted at the locus of the mic1 and mic3 genes.

the steps A, B and C can be performed in an order A followed by B followed by C, or A followed by C followed by B, or B followed by A followed by C, or B followed by C followed by A, or C followed by A followed by B, or C followed by A followed by B, or C followed by B followed by A.

This invention also concerns a process for obtaining a mutant strain of Sarcocystidae in which the mic1 and mic3 genes are deleted and in which heterologous DNA is integrated into the locus of the deleted mic1 gene or the locus of the deleted mic3 gene, from a wild strain, comprising:
a step A comprising or consisting of deletion of the mic3 gene by homologous recombination and insertion of a selection cassette framed by two identical specific recombination sites, followed by excision of said selection cassette by cre-recombinase, following which only one of the two specific recombination sites framing the cassette remains integrated at the locus of said deleted mic3 gene,
a step B comprising or consisting of deletion of the mic1 gene by homologous recombination and insertion of a selection cassette framed by two identical specific recombination sites, followed by excision of said selection cassette by cre-recombinase, following which only one of the two specific recombination sites framing the cassette remains integrated at the locus of said deleted mic1 gene

the two specific recombination sites framing the selection cassette inserted at the mic1 gene being different from the two specific recombination sites framing the selection cassette inserted at the mic3 gene the steps A and B can be carried out in an order A followed by B or B followed by A, then
a step D comprising or consisting of the targeted insertion of a heterologous DNA flanked by a specific recombination site, said heterologous DNA is integrated at the locus of the deleted mic1 gene when the specific recombination site that flanks it corresponds to the specific recombination site located at the locus of the deleted mic1 gene,

said heterologous DNA is integrated into the locus of the deleted mic3 gene when the specific recombination site that flanks it corresponds to the specific recombination site located at the locus of the deleted mic3 gene

once integrated, said heterologous DNA will therefore be framed by two specific recombination sites.

This invention also concerns a process for obtaining a mutant Sarcocystidae strain in which the mic1, mic3 and rop16I genes are deleted and in which heterologous DNA is integrated into the deleted mic1 gene or the deleted mic3 gene and a gene encoding the GRA15II protein is integrated into the deleted rop16I gene, from a wild strain, comprising:
A step A comprising or consisting of deletion of the mic3 gene by homologous recombination and insertion of a first selection cassette framed by two identical specific recombination sites, followed by excision of said selection cassette by cre-recombinase, following which only one of the two specific recombination sites framing the cassette remains integrated at the locus of said deleted mic3 gene,
A step B comprising or consisting of deletion of the mic1 gene by homologous recombination and insertion of a selection cassette framed by two identical specific recombination sites, followed by excision of said selection cassette by cre-recombinase, following which only one of the two specific recombination sites framing the cassette remains integrated at the locus of said deleted mic1 gene

the two specific recombination sites framing the selection cassette inserted at the mic1 gene being different from the two specific recombination sites framing the selection cassette inserted at the mic3 gene
a step C comprising or consisting of deletion of the rop16I gene by homologous recombination and insertion of a cassette comprising a selection cassette framed by two specific recombination sites, these two specific recombination sites being different from the two specific recombination sites framing the selection cassette inserted at the mic1 gene and those framing the selection cassette inserted at the mic3 gene, and comprising downstream of this selection cassette a sequence coding for the gra15II gene,
followed by excision of said selection cassette by cre-recombinase, following which said sequence encoding the gra15II gene is then integrated at the locus of the rop16I gene deleted downstream of a single specific recombination site

the steps A, B and C can be performed in an order A followed by B followed by C, or A followed by C followed by B, or B followed by A followed by C, or B followed by C followed by A, or C followed by A followed by B, or C followed by A followed by B, or C followed by B followed by B followed by A, then
a step D comprising or consisting of the targeted insertion of a heterologous DNA flanked by a specific recombination site, said heterologous DNA is integrated at the locus of the deleted mic1 gene when the specific recombination site that flanks it corresponds to the specific recombination site located at the locus of the deleted mic1 gene,
said heterologous DNA is integrated into the locus of the deleted mic3 gene when the specific recombination site that flanks it corresponds to the specific recombination site located at the locus of the deleted mic3 gene,
said heterologous DNA is integrated into the locus of the deleted rop16 gene when the specific recombination site that flanks it corresponds to the specific recombination site located at the locus of the deleted rop16 gene,
once integrated, said heterologous DNA will therefore be framed by two specific recombination sites.

### DESCRIPTION OF THE FIGURES

FIG. 1: this figure is a schematic representation of recombination by the Cre/LoxP system applied to the X gene framed by identical LoxP sites.

FIG. 2-A: This figure is a schematic representation of the plasmid pNcMIC3-KO-CAT-GFP LoxN. This 10063 base pair plasmid includes the selection gene encoding the chimeric protein CAT-GFP under the control of the promoter of α-tubulin of Toxoplasma gondii to allow expression of the gene in the parasite. This selection cassette is framed by two identical LoxN sites of the same orientation, added by PCR. On either side of the cassette were inserted the homologous regions flanking the ncmic3 gene.

FIG. 2-B: This figure is a schematic representation of the plasmid pNcMIC1-KO-CAT-GFP LoxP. This plasmid of 10069 base pairs includes the selection gene encoding the chimeric protein CAT-GFP under the control of the promoter of α-tubulin of Toxoplasma gondii to allow the expression of the gene in the parasite. This selection cassette is framed by two identical LoxP sites of the same orientation, added by PCR. On either side of the cassette were inserted the homologous regions flanking the ncmic1 gene.

FIG. 2-C: This figure is a schematic representation of the plasmid pTSAG1-Cre Recombinase. This 4,694 base pair plasmid includes the gene encoding the CRE-Recombinase protein under the control of the promoter of the sag1 gene of Toxoplasma gondii to allow the expression of the gene in the parasite. Downstream of the gene encoding Cre Recombinase, the 3′ UTR sequence of the sag1 gene of Toxoplasma gondii is inserted to stabilize the mRNA encoding the Cre Recombinase protein.

FIG. 3-A: This figure illustrates the 4 steps to obtain the Neo ncmic1-3 KO 2G strain. The first homologous recombination step allows the integration of the gene encoding the CAT-GFP enzyme at the mic3 gene. Selection by chloramphenicol allows the simple mutant strain Neo ncmic3 KO to be amplified. In a second step, the gene encoding the CAT-GFP protein is deleted by action of Cre Recombinase. The third step allows the integration of the gene encoding the CAT-GFP enzyme at the mic1 gene. Selection by chloramphenicol amplifies the simple mutant strain Neo ncmic1-3 KO. Finally, in a final step, the gene encoding the CAT-GFP protein is deleted by the action of Cre Recombinase.

FIG. 3-B: This figure represents the electrophoretic profiles of the PCR products obtained respectively with the wild strain NC-1 of Neospora caninum, the strain Neo ncmic3 KO, the strain Neo ncmic3 KO 2G, the strain Neo ncmic1-3 KO and the strain Neo ncmic1-3 KO 2G using the PCR primer sets in Table 3.

FIG. 3-C: This figure represents the results of the sequencing obtained on the Neo ncmic1-3 KO 2G strain and confirms that the mic1 and mic3 genes have been deleted and replaced by LoxP and LoxN scars respectively.

FIG. 4-A: This figure illustrates the immunofluorescence analysis of the detection of MIC3 protein obtained respectively with the wild Neospora caninum strain NC-1 and Neo ncmic1-3 KO 2G using an antibody specifically directed against MIC3 protein.

FIG. 4-B: This figure illustrates the immunofluorescence analysis of CATGFP protein detection obtained respectively with wild Neospora caninum strain NC-1 and mutant Neospora caninum strains using the intrinsic fluorescence of CATGFP protein. This figure shows the presence of the CATGFP protein or the absence of the CATGFP protein following the action of Cre Recombinase.

FIG. 5-A: This figure is a schematic representation of the plasmid pTgMIC3-KO-CAT-GFP LoxP. This plasmid of 9,931 base pairs includes the selection gene encoding the chimeric protein CAT-GFP under the control of the promoter of α-tubulin of Toxoplasma gondii to allow the expression of the gene in the parasite. This selection cassette is framed by two identical LoxP sites of the same orientation, added by PCR. On either side of the cassette were inserted the homologous regions flanking the tgmic3 gene.

FIG. 5-B: This figure is a schematic representation of the plasmid pTgMIC1-KO-CAT-GFP LoxN. This plasmid of 10,285 base pairs includes the selection gene encoding the chimeric protein CAT-GFP under the control of the promoter of α-tubulin of Toxoplasma gondii to allow expression of the gene in the parasite. This selection cassette is framed by two identical LoxN sites of the same orientation, added by PCR. On either side of the cassette were inserted the homologous regions flanking the tgmic1 gene.

FIG. 6-A: This figure illustrates the 4 steps to obtain the Toxo tgmic1-3 KO 2G strain. The first homologous recombination step allows the integration of the gene encoding the CAT-GFP enzyme at the mic3 gene. Selection by chloramphenicol amplifies the simple mutant strain Toxo tgmic3 KO. In a second step, the gene encoding the CAT-GFP protein is deleted by action of Cre Recombinase. The third step allows the integration of the gene encoding the CAT-GFP enzyme at the mic1 gene. Selection by chloramphenicol amplifies the simple mutant strain Toxo tgmic1-3 KO. Finally, in a final step, the gene encoding the CAT-GFP protein is deleted by the action of Cre Recombinase.

FIG. 6-B: This figure represents the electrophoretic profiles of the PCR products obtained respectively with the wild RH strain of Toxoplasma gondii, the Toxo tgmic3 KO strain, the Toxo tgmic3 KO 2G strain, the Toxo tgmic1-3 KO strain and the final Toxo tgmic1-3 KO 2G strain using the PCR primer sets in Table 7.

FIG. 6-C: This figure represents the results of the sequencing obtained on the Toxo tgmic1-3 KO 2G strain and confirms that the mic1 and mic3 genes have been deleted and replaced by LoxN and LoxP scars respectively.

FIG. 7: this figure illustrates the immunofluorescence analysis of the detection of MIC1, MIC3 or CAT-GFP proteins obtained respectively with the wild RH strain of Toxoplasma gondii, the Toxo tgmic3 KO strain, the Toxo tgmic3 KO 2G strain, the Toxo tgmic1-3 KO strain and the Toxo tgmic1-3 KO 2G strain using an antibody specifically directed against the MIC1, MIC3 protein or directly with the intrinsic fluorescent properties of the CAT-GFP protein.

FIG. 8: This figure is a schematic representation of the plasmid pTgRop16KO-Gra15IIKI-CAT-GFP Lox2272. This plasmid of 12721 base pairs includes the selection gene encoding the chimeric protein CAT-GFP under the control of the promoter of α-tubulin of Toxoplasma gondii to allow the expression of the gene in the parasite. This selection cassette is framed by two identical Lox2272 sites of the same orientation, added by PCR. Downstream of this cassette was integrated the expression cassette used to code the Gra51HAII protein. Then on either side of these cassettes were inserted the homologous regions flanking the tgrop16 gene.

FIG. 9-A: This figure illustrates the 2 steps to obtain the Toxo tgmic1-3 KO rop16 KO GRA15II KI-2G strain. The first homologous recombination step allows the integration of the gene encoding the enzyme CAT-GFP and the gene gra15IIHA at the locus of the rop16 gene. Selection by chloramphenicol amplifies the mutant strain Toxo tgmic1-3 KO rop16 KO GRA15II KI. In a second step, the gene encoding the CAT-GFP protein is deleted by action of Cre Recombinase to obtain the Toxo tgmic1-3 KO rop16 KO GRA15II KI-2G strain.

FIG. 9-B: This figure represents the electrophoretic profiles of the PCR products obtained respectively with the strains Toxo tgmic1-3 KO 2G, Toxo tgmic1-3 KO rop16 KO GRA15II KI and Toxo tgmic1-3 KO rop16 KO GRA15II KI 2G.

FIG. 9-C: This figure represents the results of the sequencing obtained on the Toxo tgmic1-3 KO rop16 KO rop16 KO GRA15II KI 2G strain and confirms that the rop16 and cat-gfp genes have been deleted and replaced by the Lox2272 scar and the gra15HAII gene.

FIG. 10: This figure illustrates the immunofluorescence analysis of the detection of the GRA15 protein (via the HA tag) obtained respectively with the strains Toxo tgmic1-3 KO rop16 KO GRA15II KI and Toxo tgmic1-3 KO rop16 KO GRA15II KI-2G using an antibody specifically directed against the HA tag. This figure also illustrates the removal of the CATGFP cassette with the disappearance of GFP fluorescence for the Toxo tgmic1-3 KO rop16 KO GRA15II KI-2G strain.

FIG. 11-A: This figure illustrates the position of the nucleic primers used in this invention to detect the presence of the three genes ncmic3, ncmic1, cat-gfp in wild strains and/or mutant strains of Neo ncmic3 KO and/or Neo ncmic3 KO 2G and/or Neo ncmic1-3 KO and/or Neo ncmic1-3 KO 2G from Neospora caninum. The digits represent the numbering of the primer sequences (SEQ ID NO: x) defined in this application.

FIG. 11-B: This figure illustrates the position of the nucleic primers used in this invention to detect the presence of the three genes tgmic3, tgmic1, cat-gfp in wild strains and/or mutant strains of Toxo tgmic3 KO and/or Toxo tgmic3 KO 2G and/or Toxo tgmic1-3 KO and/or Toxo tgmic1-3 KO 2G from Toxoplasma gondii. The digits represent the numbering of the primer sequences (SEQ ID NO: x) defined in this application.

FIG. 11-C: This figure illustrates the position of the nucleic primers used in this invention to detect the presence of the three genes tgrop16, gra15II, cat-gfp in wild strains and/or mutant strains of Toxo tgmic1-3 KO rop16 KO Gra15II KI from Toxoplasma gondii. The digits represent the numbering of the primer sequences (SEQ ID NO: x) defined in this application.

FIG. 12: This figure illustrates the specific antibody titration against Neospora caninum in the serum of mice vaccinated 30 days previously with the NeoKO 2G strain. The vehicle having been used in control. n=2 per group. ANOVA two-way . . . .

FIG. 13-A: This figure illustrates the follow-up of clinical scores observed for mice.

FIG. 13-B: This figure illustrates the specific antibody titration against T. gondii in mouse serum at D-2, J28 and J129. ANOVA one way (****: p<0.0001, *: p<0.05).

FIG. 13-C: This figure illustrates the dosage of IFNγ following a restimulation of splenocytes with the total extract of T. gondii 72 hours after restimulation. ANOVA one way (****: p<0.0001, *: p<0.05). Batch 1 corresponds to the control group, batch 2 corresponds to the challenge group 76K, batch 3 corresponds to the Toxo tgmic group 1-3 KO 2G and batch 4 corresponds to the Toxo tgmic group 1-3 KO 2G+challenge 76K.

FIG. 13-D: This figure illustrates the number of brain cysts detected in mothers. ANOVA one way (*: p<0.05). Batch 1 corresponds to the control group, batch 2 corresponds to the challenge group 76K, batch 3 corresponds to the Toxo tgmic group 1-3 KO 2G and batch 4 corresponds to the Toxo tgmic group 1-3 KO 2G+challenge 76K.

FIG. 13-E: This figure illustrates the prolificity obtained for each batch.

FIG. 13-F: This figure illustrates the sex ratio obtained for each batch. Two-way ANOVA (**: p<0.01), *: p<0.05).

FIG. 13-G: This figure illustrates the results obtained for mortality.

FIG. 13-H: This figure illustrates the results obtained for observed mortality by litter for each batch. ANOVA one way (****: p<0.0001, ***: p<0.001). Batch 1 corresponds to the control group, batch 2 corresponds to the challenge group 76K, batch 3 corresponds to the Toxo tgmic group 1-3KO 2G and batch 4 corresponds to the Toxo tgmic group 1-3 KO 2G+challenge 76K.

FIG. 13-I: This figure illustrates the percentage of survival obtained for mice over 32 days. Log-rank (Mantel-Cox) test (****: p<0.0001). Batch 1 corresponds to the control group, batch 2 corresponds to the challenge group 76K, batch 3 corresponds to the Toxo tgmic group 1-3 KO 2G and batch 4 corresponds to the Toxo tgmic group 1-3 KO 2G+challenge 76K.

FIG. 13-J: This figure illustrates the follow-up of clinical scores observed for mice.

FIG. 13-K: This figure illustrates the specific IgM titration directed against T. gondii in the mouse serum of each batch. ANOVA one way (****: p<0.0001, **: p<0.01).

### EXAMPLE 1: CONSTRUCTION OF THE NEO NCMIC1-3 KO-2G STRAIN

Haploidy of the Neospora caninum genome during the proliferative phase allows the invalidation of a gene into a single homologous recombination.

Cultivation of Parasites

All tachyzoites of the Neospora caninum strain used were produced as human fibroblasts (HFF Hs27 ATCC CRL-1634) grown in a minimal Dulbecco medium (DMEM) supplemented with 10% fetal calf serum (SVF), 2 mM glutamine, 100 U/mL penicillin and 100 U/mL streptomycin. They were collected after mechanical lysis of the host cells by 3 passages in 25G syringes.

Plasmid Construction

Plasmid pNcMIC3-KO-CAT-GFP LoxN

The plasmid pNcMic3KO-CAT-GFP LoxN of SEQ ID NO: 1 contains a CAT-GFP selection cassette SEQ ID NO: 6 comprising the cat-gfp selection gene SEQ ID NO: 2 encoding the fusion protein CAT-GFP allowing both chloramphenicol resistance (CAT) and green fluorescence (GFP: Green Fluorescent Protein), under the control of the promoter of α-tubulin of Toxoplasma gondii SEQ ID NO: 3 to allow the expression of the gene in the parasite. Downstream of the cat-gfp gene SEQ ID: 2, the sequence 3′ UTR of the sag1 gene of Toxoplasma gondii SEQ ID NO: 4 is inserted. The objective of this sequence is to stabilize the mRNA encoding the CAT/GFP fusion protein. This SEQ ID NO: 6 selection cassette is framed by two identical SEQ ID NO: 5 LoxN sites of the same orientation.

To obtain this plasmid, the CAT-GFP selection cassette SEQ ID NO: 6 was amplified by PCR on the plasmid pT230 CAT-GFP SEQ ID NO: 9 with the cat-gfp LoxN For SEQ ID NO: 7 and catgfp LoxN rev SEQ ID NO: 8 (2380pb), digested by the restriction enzymes ClaI and XbaI (2348 bp) and cloned in the plasmid pNcMic3KO-DHFR SEQ ID NO: 10 digested by ClaI and XbaI (7715 bp).

The plasmid then obtained is the plasmid pNcMic3KO-CAT-GFP LoxN of SEQ ID NO: 1.

The sequences of the primers are shown in Table 1 below. The plasmid pNcMic3KO-CAT-GFP-GFP LoxN therefore contains a CAT-GFP cassette SEQ ID NO: 6 framed by a 5′ HR sequence of the ncmic3 gene SEQ ID NO: 98 and a 3′ HR sequence of the ncmic3 gene SEQ ID NO: 97.

TABLE 1
List of primers used for the construction
of the plasmid pNcMIC3-KO-CAT-GFP LoxN

| | | |
|---|---|---|
| catgfp loxN For | SEQ ID NO: 7 | ClaI |
| | TCCGCTCTCAGAGAATCGAT | LoxN |
| | GAAGCTTATAACTTCGTATA | |
| | GTATACCTTATACGAAGTTA | |
| | TGATATGCATGTCCGCGTTC | |
| | GTGAAATCTC | |
| catgfp loxN rev | SEQ ID NO: 8 | XbaI |
| | ATACGTAAACTTCTAGATCC | loxN |
| | ATAACTTCGTATAAGGTATA | |
| | CTATACGAAGTTATCCCTCG | |
| | GGGGGGCAAGAATTGTGTTA | |
| | ACCGGTTCGA | |

Plasmid pNcMIC1-KO-CAT-GFP Lox

The plasmid pNcMic1KO-CAT-GFP LoxP of SEQ ID NO: 11 contains a CAT-GFP selection cassette SEQ ID NO: 6 comprising the cat-gfp selection gene SEQ ID NO: 2 encoding a CAT-GFP fusion protein allowing both chloramphenicol resistance (CAT) and green fluorescence (GFP: Green Fluorescent Protein), under the control of the promoter of α-tubulin of Toxoplasma gondii SEQ ID NO: 3 to allow the expression of the gene in the parasite. Downstream of the CAT-GFP coding sequence, the 3′ UTR sequence of the sag1 gene of Toxoplasma gondii SEQ ID NO: 4 is inserted. The objective of this sequence is to stabilize the mRNA encoding the CAT/GFP fusion protein. This SEQ ID NO: 6 selection cassette is framed by two identical LoxP sites SEQ ID NO: 12 of the same orientation.

To obtain this plasmid, the CAT-GFP selection cassette SEQ ID NO: 6 was amplified by PCR on the plasmid pT230 CAT-GFP SEQ ID NO: 9 with CN10 primers SEQ ID NO: 13 and CN11 SEQ ID NO: 14 allowing the addition of the LoxP sites SEQ ID NO: 12 (2394 bp), digested by the restriction enzymes HindIII and BamHI (2339 bp) and cloned in the plasmid pT230-ble SEQ ID NO: 37 digested by HindIII and BamHI (2931 bp). The plasmid then obtained is the plasmid pT230 CAT-GFP LoxP of SEQ ID NO: 113.

The 3 HR region of the ncmic1 gene was amplified by PCR from the genomic DNA of Neospora caninum strain NC-1. For amplification, the 3 HR NCmic1 F KpnI and 3 HR NCmic1 R HindIII primers (SEQ ID NO: 114 and SEQ ID NO: 115) allow the amplification of the 3 HR region of the ncmic1 gene and the creation of two restriction sites that were used to clone the 3HR fragment upstream of the CAT-GFP selection cassette in pT230 CAT-GFP LoxP of SEQ ID NO: 113. The plasmid obtained is called pNcmic1-3HR CATGFP LoxP SEQ ID NO: 118.

The 5 HR region of the ncmic1 gene was amplified by PCR from the genomic DNA of Neospora caninum strain NC-1. For amplification, the 5 HR NCmic1 F BamHI and 5 HR NCmic1 R NotI primers (SEQ ID NO: 116 and SEQ ID NO: 117) allow the amplification of the 5′ UTR region of the ncmic1 gene and the creation of two restriction sites that were used to clone the 5HR fragment downstream of the CAT-GFP selection cassette in the plasmid pNcmic1-3HR CATGFP LoxP of SEQ ID NO: 118 (BamHI-NotI).

The plasmid then obtained is the plasmid pNcMic1KO-CAT-GFP LoxP of SEQ ID NO: 11. pNcMic1KO-CAT-GFP LoxP therefore contains a CAT-GFP cassette SEQ ID NO: 6 framed by a 5′ HR sequence of the ncmic1 gene SEQ ID NO: 96 and a 3′ HR sequence of the ncmic1 gene SEQ ID NO: 95.

The sequences of the primers are shown in Table 2 below.

TABLE 2
list of primers used for the construction
of the plasmid pNcmic1 KO CATGFP LoxP
Construction of the plasmid pNcmic1
KO CATGFP LoxP

| | | |
|---|---|---|
| CN10 | SEQ ID NO: 13 | HindIII, |
| | GCGGCCAAGCTTATAACTT | loxP |
| | CGTATAATGTATGCTATAC | |
| | GAAGTTATGATATGCATGT | |
| | CCGCgttcgtgaaatctct | |
| | gatcaagcgg | |
| CN11 | SEQ ID NO: 14 | SpeI, |
| | cgacgcacgctgtcactca | BamHI, |
| | acttgctGCTAGAACTAGT | loxP |
| | GGATCCATAACTTCGTATA | |
| | GCATACATTATACGAAGTT | |
| | ATCCCTCGGGGGGGCAAGA | |
| | ATT | |
| 3 HR | SEQ ID NO: 114 | KpnI |
| NCmic1 F | CGCGGTACCAGGCAGAAGT | |
| KpnI | AAAGAAGGTTCCTC | |
| 3 HR | SEQ ID NO: 115 | HindIII |
| NCmic1 R | CGCAAGCTTTGATCACGCA | |
| HindIII | AGAAAAGAAGC | |
| 5 HR | SEQ ID NO: 116 | BamHI |
| NCmic1 F | CGCGGATCCCATTTGTAGA | |
| BamHI | TACGGTTGCACAC | |
| 5 HR | SEQ ID NO: 117 | NotI |
| NCmicl R | CGCGCGGCCGCACATTCAG | |
| Notl | ACGGCAGAACTCTG | |

Plasmid pTSAG1-Cre Recombinase

The plasmid pT-SAG1-Cre-Recombinase SEQ ID NO: 15 was constructed to transiently express in the parasite Toxoplasma gondii and its genetically modified derivatives the gene encoding the Cre Recombinase protein derived from bacteriophage P1 (Brecht et al., 1999, same reference as above).

The plasmid pT-SAG1-Cre-Recombinase SEQ ID NO: 15 is derived from the plasmid pUC18 (commercial plasmid), which contains a cassette for the expression of Cre Recombinase from bacteriophage P1.

In the plasmid pT-SAG1-Cre-Recombinase SEQ ID NO: 15, the gene encoding the Cre Recombinase SEQ ID NO: 16 is placed under the dependence of the promoter of the sag1 gene of Toxoplasma gondii SEQ ID NO: 17, which allows the expression of the Cre Recombinase protein in transfected Toxoplasma gondii parasites. Downstream of the gene encoding Cre Recombinase, the 3′ UTR sequence of the sag1 gene of Toxoplasma gondii SEQ ID NO: 4 is inserted. The objective of this sequence is to stabilize the mRNA encoding the Cre Recombinase protein.

The absence of a specific selection cassette does not allow a stable integration of the transfected genetic material but only a transient expression of Cre Recombinase.

Construction of the Neomic1-3 KO-2G Strain

The construction of the second generation attenuated live strain, called Neo ncmic1-3 KO-2G, is done in 4 distinct steps:
1. Deletion by homologous recombination of the ncmic3 gene and its replacement by the CAT-GFP selection cassette SEQ ID NO: 6 framed by LoxN sequences SEQ ID NO: 5.
   To obtain this strain, the wild strain NC-1 of Neospora caninum is electroporated with the plasmid pNcMIC3-KO-CAT-GFP LoxN of SEQ ID NO: 1. 20 μg of plasmid purified and linearized by KpnI are added to 10⁷ parasites suspended in the CYTOMIX electroporation medium containing ATP (3 mM) and Glutathione (3 mM) (Van den Hoff et al, Nucleic Acid Research, June 11; 20(11):2902), and electroporation was performed in a 4 mm gap cell, in a volume of 800 μL on a BioRad device (Parameters: 2000V, 50 ohms, 25 μF, with two electric shocks). After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing DNA genomic DNA extraction from the clones of interest. The strain obtained is called Neo ncmic3 KO. In this strain, the mic3 gene has been deleted and replaced by the CATGFP selection cassette SEQ ID NO: 6. Thus, the theoretical sequence at the locus of the deleted mic3 gene containing the CATGFP selection cassette is SEQ ID NO: 101. Since the mic1 gene is not deleted, the sequence of the mic1 gene SEQ ID NO: 106 is present in the genome of the strain.
2. Deletion of the selection cassette SEQ ID NO: 6: the obtained Neo ncmic3 KO strain is electroporated with the plasmid pT-SAG1-CRE-Recombinase SEQ ID NO: 15.
   The transiently expressed enzyme will allow the excision of the CAT-GFP selection cassette SEQ ID NO: 6. The electroporation protocol is similar to the previous one. After electroporation, the tachyzoites are deposited on a monolayer of HFF cells in culture. After 2 to 7 days of culture, the parasites are subcloned in 96-well plate on a monolayer of HFF cells and the clones of interest are identified by PCR after performing DNA genomic DNA extraction from the clones of interest. The strain obtained is called Neo ncmic3 KO-2G.
   In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 18. Since the mic1 gene is not deleted, the sequence of the mic1 gene SEQ ID NO: 106 is present in the genome of the strain.
3. Deletion by homologous recombination of the ncmic1 gene and its replacement by the CAT-GFP selection cassette SEQ ID NO: 6 framed by LoxP sequences SEQ ID NO: 12.
   To obtain this strain, the Neo ncmic3 KO-2G strain is electroporated with the plasmid pNcMIC1-KO-CAT-GFP LoxP of SEQ ID NO: 11 linearized by KpnI, according to the protocol previously described. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Neo ncmic1-3 KO.
   In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 18. In this strain, the mic1 gene has been deleted and replaced by the CATGFP selection cassette SEQ ID NO: 6, so the theoretical sequence at the locus of the deleted mic1 gene containing the CATGFP selection cassette is SEQ ID NO: 102.
4. Deletion of the selection cassette SEQ ID NO: 6: the obtained Neo ncmic1-3 KO strain is electroporated with the plasmid pT-SAG1-CRE-Recombinase SEQ ID NO: 15.
The transiently expressed enzyme will allow the excision of the CAT-GFP selection cassette SEQ ID NO: 6. After electroporation, the tachyzoites are deposited on a monolayer of HFF cells in culture. After 2 to 7 days of culture, the parasites are subcloned in 96-well plate on a monolayer of HFF cells and the clones of interest are identified by PCR after performing DNA genomic DNA extraction from the clones of interest. The strain obtained is called Neo ncmic1-3 KO-2G.
   In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 18.
   In this strain, the mic1 gene was deleted and the CATGFP selection cassette SEQ ID NO: 6 was excised. Thus, the theoretical sequence at the locus of the deleted mic1 gene containing a single LoxP site SEQ ID NO: 12, is SEQ ID NO: 91.

Validation of the Neo Ncmic1-3 KO-2G Strain by PCR

To validate the Neo ncmic1-3 KO-2G strain, PCR analyses are carried out using genomic DNA extracted with DNAzol from a parasitic pellet composed of 10⁷ parasites. The primers used for PCR are described in FIG. 11-A and are detailed in Table 3 below. PCR products are analyzed by agarose gel electrophoresis (FIG. 3-B). Their expected and observed sizes are also described in Table 4 below. For more clarity on FIG. 3-B, only the 3 steps of the realization are represented (ncmic3, ncmic3KO CATGFP and ncmic3KO LoxN (2G) or ncmic1, ncmic1KO CATGFP and ncmic1KO LoxP(2G)), the results obtained are in accordance with the expected sizes

TABLE 3
List of primers used for strain validation.

| | | Primer F | Sequence | | Primer R | Sequence |
|---|---|---|---|---|---|---|
| Ncmic3 | c | Nc mic3 F | SEQ ID NO: 22 | d | Nc mic3 R | SEQ ID NO: 23 |
| (mix1) | | | TTTCCCTTCTAAA | | | CCTTCAGTGGTTT |
| | | | CACAGTCG | | | TCTCCATGAGT |
| Validation | i | Integ | SEQ ID NO: 24 | j | ORF | SEQ ID NO: 25 |
| KO | | NCmic3 F | GAAAGTGTCAGTG | | CATGFP R | CCGTTTGGTGTGG |
| 5′Ncmic3 | | | GTAGAGACTGC | | | ATGTCTCTCTCT |
| (mix2) | | | | | | |
| Validation | k | ORF | SEQ ID NO: 26 | l | Integ | SEQ ID NO: 27 |
| KO | | CATGFP F | GCATCGACTTCAA | | NCmic3 R | TGTTTACAGGTCA |
| 3′Ncmic3 | | | GGAGGAGGAC | | | TCCAGAAAAGG |
| (mix3) | | | | | | |
| Scar | g | AF3 | SEQ ID NO: 32 | h | AF4 | SEQ ID NO: 33 |
| Ncmic3 | | | GTCATCGACCGCC | | | GCAGAGAGGTTCT |
| (mix4) | | | GCCGGAACTAGTA | | | GCGTATCTAACAC |
| | | | GT | | | ACGG |
| Ncmic1 | a | Nc mic1 F | SEQ ID NO: 20 | b | Nc mic1 R | SEQ ID NO: 21 |
| (mix5) | | | ACCCGGAGAATTA | | | TTCTCCAGGCACT |
| | | | TCGCCTA | | | CACCTCACCT |
| Validation | m | Integ | SEQ ID NO: 28 | j | ORF | SEQ ID NO: 25 |
| KO | | NCmic1 F | CCGAGCAAGTTAG | | CATGFP R | CCGTTTGGTGTGG |
| 5′Ncmic1 | | | CAAGTTAGCAAGT | | | ATGTCTCTCTC |
| (mix6) | | | CC | | | |
| Validation | k | ORF | SEQ ID NO: 26 | n | Integ | SEQ ID NO: 29 |
| KO | | CATGFP F | GCATCGACTTCAA | | NCmic1 R | CTTGTGTCCGTCA |
| 3′Ncmic1 | | | GGAGGAGGAC | | | CATCGTTTG |
| (mix7) | | | | | | |
| Scar | e | ncmic1creLox | SEQ ID NO: 30 | f | ncmic1creLox | SEQ ID NO: 31 |
| Ncmic1 | | F B B | GGCGACATACTGC | | R B B | GATCGGAGTCTCT |
| (mix8) | | | ATTGGAT | | | GTCCCTCAA |

TABLE 4
Expected amplicon size (in base pairs) of the different
PCRs for validation of the construction of KO
strains of Neospora caninum

| | | Neo | Neo | Neo | Neo |
|---|---|---|---|---|---|
| | | ncmic3 | ncmic3 | ncmic1-3 | ncmic1-3 |
| | NC-1 | KO | KO LoxN | KO LoxP | KO 2G |
| Ncmic3 (mix1) | 850 | — | — | — | — |
| Validation KO | — | 2960 | — | — | — |
| 5′Ncmic3 (mix2) | | | | | |
| Validation KO | — | 3668 | — | — | — |
| 3′Ncmic3 (mix3) | | | | | |
| Scar Ncmic3 (mix4) | 2163 | 2575 | 276 | 276 | 276 |
| Ncmic1 (mix5) | 701 | 701 | 701 | — | — |
| Validation KO | — | — | — | 3359 | — |
| 5′Ncmic1 (mix6) | | | | | |
| Validation KO | — | — | — | 3421 | — |
| 3′Ncmic1 (mix7) | | | | | |
| Scar Ncmic1 (mix8) | 3161 | 3161 | 3161 | 2560 | 261 |

The electrophoresis of the PCR products performed with the primers c SEQ ID NO: 22 and d SEQ ID NO: 23 (mix 1) allow to highlight a band with 850 base pairs for the NC-1 strain, in accordance with the expected band size and confirming the presence of the ncmic3 gene SEQ ID NO: 105 in this strain. This band is not detected in the strains Neo ncmic3 KO, Neo ncmic3 KO-2G, Neo ncmic1-3 KO and Neo ncmic1-3 KO-2G confirming the suppression of the gene in these strains.

The electrophoresis of the PCR products carried out with the primers i SEQ ID NO: 24 and j SEQ ID NO: 25 and the primers k SEQ ID NO: 26 and SEQ ID NO: 27 (mix Ncmic3) allow to highlight respectively a band at 2960 and 3668 base pairs for the Neo ncmic3 KO strain, according to the expected band size and confirming the presence of the cat-gfp selection gene in this strain instead of the ncmic3 gene. This band is not detected in NC-1, Neo ncmic3 KO-2G, Neo ncmic1-3 KO and Neo ncmic1-3 KO-2G strains confirming the absence of the cat-gfp selection gene SEQ ID NO: 2 at the ncmic 3 locus in these strains.

The electrophoresis of the PCR products made with the primers g SEQ ID NO: 32 and h SEQ ID NO: 33 (Ncmic3 scar) allow to highlight different bands according to the expected band size. A band with 2163 base pairs for the NC-1 strain confirms the presence of the ncmic3 gene SEQ ID NO: 105 in this strain. A tape of 2575 base pairs is highlighted for the Neo ncmic3 KO strain confirming the presence of the CATGFP selection cassette SEQ ID NO: 6 in place of the ncmic3 gene. Finally, a band of 276 base pairs is highlighted for the strains Neo ncmic3 KO-2G Neo ncmic1-3 KO and Neo ncmic1-3 KO-2G confirming the deletion of the cat-gfp selection gene SEQ ID NO: 2 in these strains, leaving a LoxN scar SEQ ID NO: 5 in the genome.

The electrophoresis of the PCR products carried out with the primers a SEQ ID NO: 20 and b SEQ ID NO: 21 (mix Ncmic1) allow to highlight a band with 644 base pairs for the strains NC-1, Neo ncmic3 KO, Neo ncmic3 KO-2G, according to the expected band sizes and confirming the presence of the gene ncmic1 SEQ ID NO: 106 in these strains. These bands are not detected in the strains Neo ncmic1-3 KO and Neo ncmic1-3 KO-2G confirming the suppression of the gene in these strains.

The electrophoresis of the PCR products carried out with the primers m SEQ ID NO: 28 and j SEQ ID NO: 25 and the primers k SEQ ID NO: 26 and n SEQ ID NO: 29 (mix Ncmic1) allow to highlight respectively a band at 3359 and 3421 base pairs for the Neo ncmic1-3 KO strain, according to the expected band size and confirming the presence of the cat-gfp selection gene SEQ ID NO: 2 in this strain instead of the ncmic1 gene. This band is not detected in the wild NC-1 strains of N. caninum, Neo ncmic3 KO, Neo ncmic3 KO-2G, and Neo ncmic1-3 KO-2G confirming the absence of the at-gfp selection gene SEQ ID NO: 2 at locus ncmic 1 in these strains.

The electrophoresis of the PCR products made with the primers SEQ ID NO: 30 and f SEQ ID NO: 31 (scar Ncmic1) allow to highlight different bands according to the expected band size. A band of 2182 base pairs for the strains NC-1, Neo ncmic3 KO, Neo ncmic3 KO-2G confirms the presence of the ncmic1 gene in these strains. A tape of 2560 base pairs is highlighted for the Neo ncmic1-3 KO strain confirming the presence of the CATGFP selection cassette SEQ ID NO: 6 in place of the ncmic1 gene. Finally, a band of 261 base pairs is highlighted for the Neo ncmic1-3 KO-2G strain confirming the deletion of the cat-gfp selection gene SEQ ID NO: 2 in this strain, leaving a LoxP scar SEQ ID NO: 12 in the genome.

The “scar” PCR products (mix 4 and 8) were sequenced and the sequencing confirmed that:
the ncmic3 gene SEQ ID NO: 105 has been deleted and that the CAT-GFP selection cassette SEQ ID NO: 6 has been deleted by the action of the Cre-Recombinase leaving a LoxN scar SEQ ID NO: 5 in accordance with the expected sequence (FIG. 3C).
the ncmic1 gene SEQ ID NO: 106 has been deleted and that the CAT-GFP selection cassette SEQ ID NO: 6 has been deleted by the action of the Cre-Recombinase leaving a LoxP scar SEQ ID NO: 12 in accordance with the expected sequence (FIG. 3C).

Validation of the Neo Ncmic1-3 KO-2G Strain by Immunofluorescence

Tachyzoites grown 24 hours on glass lamellae covered with a monolayer of HFF cells. The infected cells were washed twice with PBS1×, and fixed with 4% formaldehyde for 30 minutes. After 3 washes in PBS1×, the infected HFF cells were permeabilized with 0.1% Triton X-100 in PBS1× for 5 min. After 3 washes with PBS 1×, a saturation step is performed with a solution of PBS 1×/SVF 10% for 30 min. The cells were then incubated with the primary antibody diluted in 2% SVF for 1 hour, washed 3 times with PBS1× and incubated with a secondary antibody diluted in 2% PBS/SVF solution for 1 hour. After 2 washes with PBS1×, the glass slides are mounted on a slide with Immu-Mount™ and observed under a fluorescence microscope.

The primary antibody Tgmic3 allows a recognition of the protein Ncmic3, it allows to detect the expression of the protein NcMIC3 SEQ ID NO: 118 in the parasite (rabbit antibody anti-mic3: rnAb anti-MIC3 s3) and the commercial secondary antibody used is Alexa Fluor® 594 goat anti rabbit (Life technologies ref. A-11012).

The results show that no fluorescence is detectable at the apical pole of the parasite, indicating the absence of MIC3 proteins (FIG. 4-A).

The primary antibody Tgmic 1 does not allow recognition of the NcMIC1 protein SEQ ID NO: 119.

The results show that the parasites express a green fluorescent reflecting the expression of the CATGFP protein SEQ ID NO: 120 following the realization of gene deletions (Neo ncmic3 KO and Neo ncmic1-3 KO) while after the action of recombinase Cre, the Neo ncmic3 KO-2G and Neo ncmic1-3 KO-2G strains are no longer fluorescent (removal of the CATGFP cassette SEQ ID NO: 6) (FIG. 4-B).

### EXAMPLE 2: CONSTRUCTION OF THE TOXO TGMIC1-3 KO-2G STRAIN

Haploidy of the Toxoplasma gondii genome during the proliferative phase allows the invalidation of a gene into a single homologous recombination.

Cultivation of Parasites

All tachyzoites of the Toxoplasma gondii strain used were produced in human fibroblasts (HFF Hs27 ATCC CRL-1634) grown in a minimal Dulbecco medium (DMEM) supplemented with 10% fetal calf serum (SVF), 2 mM glutamine, 100 U/mL penicillin and 100 U/mL streptomycin. They were collected after mechanical lysis of the host cells by 3 passages in 25G syringes.

Plasmid Construction

Plasmid pTgMIC3-KO-CAT-GFP LoxP

The plasmid pTgMIC3-KO-CAT-GFP LoxP of SEQ ID NO: 34 (FIG. 5A) was constructed to remove the gene encoding the TgMIC3 protein from Toxoplasma gondii (ATCC reference: RH Pra310 strain) by homologous recombination.

The plasmid pTgMic3KO-CAT-GFP LoxP of SEQ ID NO: 34 contains a CAT-GFP selection cassette SEQ ID NO: 6 comprising a cat-gfp selection gene SEQ ID NO: 2 encoding a CAT-GFP fusion protein allowing both chloramphenicol (CAT) resistance and green fluorescence (GFP: Green Fluorescent Protein), under the control of the promoter of α-tubulin of Toxoplasma gondii SEQ ID NO: 3. Downstream of the cat-gfp gene SEQ ID NO: 2, the sequence 3′ UTR of the sag1 gene of Toxoplasma gondii SEQ ID NO: 4 is inserted. SEQ ID NO: 6 was amplified from the plasmid pT230 CAT-GFP SEQ ID NO: 9 using the primers SEQ ID NO: 35 and SEQ ID NO: 36 which allow the addition of LoxP sites (SEQ ID NO: 12) and HindIII and SpeI restriction sites. The nucleotide sequence amplified by PCR (2389 bp) was then cloned in the plasmid pT230TUB Ble SEQ ID NO: 37 by enzymatic digestion with the restriction enzymes HindIII and SpeI. The plasmid obtained is called pCATGFP LoxP of SEQ ID NO: 38.

The 3′ HR region of the tgmic3 gene SEQ ID NO: 39 was obtained by the double enzymatic digestion of the plasmid pmic3KO-2 SEQ ID NO: 40 with the restriction enzymes KpnI and HindIII (2145pb), then cloned in the plasmid pCATGFP LoxP of SEQ ID NO: 38 digested by KpnI and HindIII (5238pb). The plasmid obtained is called p3′ UTRmic3-CATGFP LoxP of SEQ ID NO: 41.

The 5′HR region of the tgmic3 gene SEQ ID NO: 42 was amplified by PCR from the genomic DNA of the RH strain of T. gondii. For amplification, the primers SEQ ID NO: 43 and SEQ ID NO: 44 allow the amplification of the 5′ UTR region of the tgmic3 gene and the creation of two restriction sites (2568pb/SpeI and XbaI sites). These restriction sites were used to clone the 5HR fragment digested by SpeI and XbaI (2548pb) into the plasmid p3′ UTRmic3-CATGFP LoxP of SEQ ID NO: 38 downstream of the CAT-GFP selection cassette SEQ ID NO: 6 at the SpeI site of the plasmid previously described (7383pb) to obtain the plasmid pTgMic3KO-CAT-GFP LoxP of SEQ ID NO: 34.

The sequences of the primers are shown in Table 5 below.

TABLE 5
Sequences of primers used for the construction of
the plasmid pTgMIC3-KO-CAT- GFP LoxP.
Construction of the plasmid pTgmic3KO CAT-GFP LoxP

| | | |
|---|---|---|
| SEQ ID NO: 35 | GCGGCCAAGCTTATAACTTCGTA | HindIII, |
| | TAATGTATGCTATACGAAGTTAT | loxP |
| | GATATGCATGTCCGCgttcgtga | |
| | aatctctgatcaagcgg | |
| SEQ ID NO: 36 | cgacgcacgctgtcactcaactt | SpeI, BamHI, |
| | gctGCTAGAACTAGTGGATCCAT | loxP |
| | AACTTCGTATAGCATACATTATA | |
| | CGAAGTTATCCCTCGG | |
| SEQ ID NO: 43 | GGGATCCACTAGTTTACGTATCG | BamHI, SpeI, |
| | CGACTAGCAGCAAGTTGAGTGAC | SnaBI, NruI |
| | AGCG | |
| SEQ ID NO: 44 | GCTGCAGTCTAGAGATATCCACG | PstI, XbaI, |
| | TGGAATTCCTCTTGGGAAGAACA | EcoRV PmlI |
| | AT | |

Plasmid pTgMIC1-KO-CAT-GFP LoxN

The plasmid pTgMIC1-KO-CAT-GFP LoxN of SEQ ID NO: 45 (FIG. 5-B) was constructed to remove the gene encoding the TgMIC1 protein from Toxoplasma gondii by homologous recombination.

The plasmid pTgMic1KO-CAT-GFP LoxN of SEQ ID NO: 45 contains a CAT-GFP selection cassette SEQ ID NO: 6 comprising the cat-gfp selection gene SEQ ID NO: 2 encoding the fusion protein CAT-GFP allowing both chloramphenicol (CAT) resistance and green fluorescence (GFP: Green Fluorescent Protein), under the control of the Toxoplasma gondii promoter α-tubulin SEQ ID NO: 3. Downstream of the cat-gfp gene SEQ ID NO: 2, the sequence 3′ UTR of the sag1 gene of Toxoplasma gondii SEQ ID NO: 4 was inserted. SEQ ID NO: 6 was amplified from the plasmid pT230 CAT-GFP SEQ ID NO: 9 using the primers SEQ ID NO: 46 and SEQ ID NO: 47 which allow the addition of LoxN sites (SEQ ID NO: 5) and ClaI and XbaI restriction sites. The nucleotide sequence amplified by PCR was then cloned in the plasmid pNcMic3KO-DHFR SEQ ID NO: 10 digested by ClaI and XbaI (7715 bp) by enzymatic digestion with the restriction enzymes ClaI and XbaI. The plasmid obtained is called pNCmic3KO CATGFP LoxN of SEQ ID NO: 48.

The 5HR tgmic1 fragment SEQ ID NO: 49 was amplified by PCR with the primers SEQ ID NO: 50 and SEQ ID NO: 51 (2364pb), the restriction sites KpnI and ClaI were added by PCR. The amplified fragment is digested by the restriction enzymes KpnI and ClaI (2337pb) and cloned in the plasmid pNCmic3KO CATGFP LoxN of SEQ ID NO: 48 (see example 1 for obtaining the plasmid pNCmic3KO CATGFP LoxN) digested by KpnI and ClaI (7740pb) to replace the 5HR ncmic3 fragment (fragment digested by KpnI and ClaI). The plasmid obtained is called pTgmic1KO5HR-NCmic3KO3KO3HR CATGFP LoxN of SEQ ID NO: 111.

Then the 3HR tgmic1 fragment SEQ ID NO: 52 was amplified by PCR with the primers SEQ ID NO: 53 and SEQ ID NO: 54 (2750pb), the restriction sites XbaI and NotI were added by PCR. The amplified fragment is digested by the restriction enzymes XbaI and NotI (2720 bp) and then cloned in the plasmid pTgmic1KO5HR-NCmic3KO3KO3HR CATGFP LoxN of SEQ ID NO: 111 digested by the restriction enzymes XbaI and NotI (7565 bp) to replace the 3HR ncmic3 fragment (fragment digested by XbaI and NotI). The plasmid obtained is called pTgmic1KO CAT-GFP LoxN of SEQ ID NO: 45.

The primers used for PCRs are detailed in Table 6 below.

TABLE 6
Primers for the construction of the plasmid
pTgmic3KO CAT-GFP LoxP
Construction of the plasmid pTgmic3KO CAT-GFP LoxP

| | | | |
|---|---|---|---|
| SEQ ID NO: 46 | TCCGCTCTCAGAGAATCGAT | ClaI | |
| | GAAGCTTATAACTTCGTATA | LoxN | |
| | GTATACCTTATACGAAGTTA | | |
| | TGATATGCATGTCCGCGTTC | | |
| | GTGAAATCTC | | |
| SEQ ID NO: 47 | ATACGTAAACTTCTAGATCC | XbaI | |
| | ATAACTTCGTATAAGGTATA | loxN | |
| | CTATACGAAGTTATCCCTCG | | |
| | GGGGGGCAAGAATTGTGTTA | | |
| | ACCGGTTCGA | | |
| SEQ ID NO: 50 | GTAGCAAGGTACCACAAGCT | | 5HR |
| | AAAGAAAGTAGTGCCTCTTC | | tgmic1For |
| | TAAA | | KpnI |
| SEQ ID NO: 51 | TAAACGGGCTGATCGATGCA | | 5HR |
| | GGTAAATTCTATAGCCGGGT | | tgmic1 |
| | | | Rev ClaI |
| SEQ ID NO: 53 | TAAACGGGCTGATCGATGCA | | 3HR |
| | GGTAAATTCTATAGCCGGGT | | tgmic1For |
| | | | XbaI |
| SEQ ID NO: 54 | AAAAACTCGGTGGCGGCCGC | | 3HR |
| | ATAAAGAAGAGCAAGGAAAA | | tgmic1rev |
| | | | NotI |

Plasmid pTSAG1-Cre Recombinase

The plasmid pT-SAG1-Cre-Recombinase SEQ ID NO: 15 was constructed to transiently express in the parasite Toxoplasma gondii and its genetically modified derivatives the gene encoding the Cre Recombinase protein derived from bacteriophage P1 (Brecht et al., 1999, same reference as above).

The plasmid pT-SAG1-Cre-Recombinase SEQ ID NO: 15 is derived from the plasmid pUC18 (commercial plasmid) which contains an expression cassette of the Cre Recombinase of bacteriophage P1. In the plasmid pT-SAG1-Cre-Recombinase SEQ ID NO: 15, the gene encoding the Cre Recombinase SEQ ID NO: 16, is placed under the dependence of the promoter of the sag1 gene of Toxoplasma gondii SEQ ID NO: 17, which allows the expression of the Cre Recombinase protein in transfected Toxoplasma gondii parasites. Downstream of the gene encoding the Cre Recombinase SEQ ID NO: 16, the 3′ UTR sequence of the sag1 gene of Toxoplasma gondii SEQ ID NO: 4 is inserted. The objective of this sequence is to stabilize the mRNA encoding the Cre Recombinase protein. The absence of a specific selection cassette does not allow a stable integration of the transfected genetic material but only a transient expression of Cre Recombinase.

Construction of the Toxo Tgmic1-3 KO-2G Strain

The construction of the second generation attenuated live strain, called Toxo tgmic1-3 KO-2G, is done in 4 distinct steps (FIG. 6-A):
1. Deletion by homologous recombination of the tgmic3 gene and its replacement by the CAT-GFP selection cassette SEQ ID NO: 6 framed by LoxP sequences SEQ ID NO: 12.
   To obtain this strain, the wild strain of Toxoplasma gondii RH (reference ATCC: PRA-310) is electroporated with the plasmid pTgMIC3-KO-CAT-GFP LoxP SEQ ID NO: 34. 20 μg of plasmid purified and linearized by KpnI are added to 10⁷ parasites suspended in the CYTOMIX electroporation medium containing ATP (3 mM) and Glutathione (3 mM) (Van den Hoff et al, Nucleic Acid Research, June 11; 20(11):2902), and electroporation was performed in a 4 mm gap cell, in a volume of 800 μL on a BioRad device (Parameters: 2000V, 50 ohms, 25 μF, with two electric shocks). After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing DNA genomic DNA extraction from the clones of interest. The strain obtained is called Toxo tgmic3 KO.
   In this strain, the mic3 gene has been deleted and replaced by the CATGFP selection cassette SEQ ID NO: 6. Thus, the theoretical sequence at the locus of the deleted mic3 gene containing the CATGFP selection cassette is SEQ ID NO: 103. Since the mic1 gene is not deleted, the sequence of the mic1 gene SEQ ID NO: 108 is present in the genome of the strain.
2. Deletion of the selection cassette SEQ ID NO: 6: the Toxo tgmic3 KO strain obtained is electroporated with the plasmid pT-SAG1-CRE-Recombinase SEQ ID NO: 15.
   The transiently expressed enzyme will allow the excision of the CAT-GFP selection cassette SEQ ID NO: 6. The electroporation protocol is similar to the previous one. After electroporation, the tachyzoites are deposited on a monolayer of HFF cells in culture. After 2 to 7 days of culture, the parasites are subcloned in 96-well plate on a monolayer of HFF cells and the clones of interest are identified by PCR after performing DNA genomic DNA extraction from the clones of interest. The strain obtained is called Toxo tgmic3 KO-2G.
3. In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 19. since the mic1 gene is not deleted, the sequence of the mic1 gene SEQ ID NO: 108 is present in the genome of the strain. Deletion by homologous recombination of the tgmic1 gene and its replacement by the CAT-GFP selection cassette SEQ ID NO: 6 framed by LoxN sequences SEQ ID NO: 5. To obtain this strain, the Toxo tgmic3 KO-2G strain is electroporated with the plasmid pTgMIC1-KO-CAT-GFP LoxN of SEQ ID NO: 45 linearized by PciI, according to the protocol previously described. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Toxo tgmic1-3 KO.
4. In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 19.
   In this strain, the mic1 gene has been deleted and replaced by the CATGFP selection cassette SEQ ID NO: 6. Thus, the theoretical sequence at the locus of the deleted mic1 gene containing the CATGFP selection cassette is SEQ ID NO: 104.
5. Deletion of the selection cassette SEQ ID NO: 6: the Toxo tgmic1-3 KO strain obtained is electroporated with the plasmid pT-SAG1-CRE-Recombinase SEQ ID NO: 15. The transiently expressed enzyme will allow the excision of the CAT-GFP selection cassette SEQ ID NO: 6. After electroporation, the tachyzoites are deposited on a monolayer of HFF cells in culture. After 2 to 7 days of culture, the parasites are subcloned in 96-well plate on a monolayer of HFF cells and the clones of interest are identified by PCR after performing DNA genomic DNA extraction from the clones of interest. The strain obtained is called Toxo tgmic1-3 KO-2G.
   In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 19.
   In this strain, the mic1 gene was deleted and the CATGFP selection cassette SEQ ID NO: 6 was excised. Thus, the theoretical sequence at the locus of the deleted mic1 gene containing a single LoxN site SEQ ID NO: 5, is SEQ ID NO: 92.

Validation of the Toxo Tgmic1-3 KO-2G Strain by PCR

To validate the Toxo tgmic1-3 KO-2G strain, PCR analyses are carried out using genomic DNA extracted with DNAzol from a parasitic pellet composed of 10⁷ parasites. The primers used for PCR are described in FIG. 11-B and detailed in Table 7 below. PCR products are analyzed by agarose gel electrophoresis (FIG. 6-B). Their expected and observed sizes are also described in Table 8 below. For greater clarity in FIG. 6-B, only the 3 steps of the realization are represented (tgmic3, tgmic3KO CATGFP and tgmic3KO LoxP (2G) or tgmic1, tgmic1KO CATGFP and tgmic1KO LoxN (2G)), the results obtained are in accordance with the expected sizes.

TABLE 7
List of primers used for strain validation.

| | | | | | | |
|---|---|---|---|---|---|---|
| Tgmic3 | 5 | TgMIC3 | SEQ ID NO 61: | 6 | TgMIC3 | SEQ ID NO 62: |
| (mix1) | | For | CCTCCGATGTGTGA | | Rev | GATCCTCCGAGCA |
| | | | CTTTTGGT | | | AGTCAAGTCAAC |
| Validation | 13 | CN58 | SEQ ID NO. 63: | j | ORF | SEQ ID NO 25: |
| KO | | | ATACGAAGGGATTC | | CATGF | CCGTTTGGTGGAT |
| 5′Tgmic3 | | | GACGTG | | PR | GTGTCTCTCT |
| (mix2) | | | | | | |
| Validation | k | ORF | SEQ ID NO. 26: | 14 | HR | SEQ ID NO 64 |
| KO | | CATGFP | GCATCGACTTCAAG | | Mic3 | ATATGCGGATGAG |
| 3′Tgmic3 | | F | GAGGAGGAC | | RL | GAGTGAGTGCTCG |
| (mix3) | | | | | | AATT |
| Scar | 9 | Tgmic3 | SEQ ID NO 65: | 10 | Tgmic3 | SEQ ID NO. 66: |
| Tgmic3 | | LoxN F | GTGTGGAGACTACT | | LoxN R | CCTCCGATGTGAC |
| (mix4) | | | TTTTTACTTCGTTA | | | TTTTGGT |
| | | | C | | | |
| Tgmic1 | 3 | TgMIC1 | SEQ ID NO 55: | 4 | TgMIC1 | SEQ ID NO 56: |
| (mix5) | | For | ATGCGCGCGCTATA | | Rev | AGAAACAACGCCT |
| | | | AAAGAATCG | | | GGCCCAT |
| Validation | 11 | tgmic1K | SEQ ID NO. 57: | j | ORF | SEQ ID NO 25: |
| KO | | O integ | GTGCGATGACGTGA | | CATGF | CCGTTTGGTGGAT |
| 5′Tgmic1 | | F | CGTGGGGCTTCTCT | | P R | GTGTCTCTCT |
| (mix6) | | | ATCATCATGTGTGT | | | |
| Validation | k | ORF | SEQ ID NO. 26: | 12 | tgmic1K | SEQ ID NO. 58: |
| KO | | CATGFP | GCATCGACTTCAAG | | O integ | GATTCGCTTCGTC |
| 3′Tgmic1 | | F | GAGGAGGAC | | R | AGTCACTTCTGGG |
| (mix7) | | | | | | GTACGGATGC |
| Scar | 7 | Tgmic1 | SEQ ID NO 59: | 8 | Tgmic1 | SEQ ID NO 60: |
| Tgmic1 | | LoxN F | CCCGTCTAGCAAGA | | LoxN R | TCGGTGCTGCTGC |
| (mix8) | | | CACCTCAAA | | | TCAGTAATTG |

TABLE 8
Expected amplicon size (in base pairs) of the different
PCRs for validation of the construction of KO
strains of Toxoplasma gondii

| | | Toxo | Toxo | Toxo | Toxo |
|---|---|---|---|---|---|
| | | tgmic3 | tgmic3 | tgmic1-3 | tgmic1-3 |
| | HR | KO | KO - 2G | KO | KO - 2G |
| Tgmic3 (mix1) | 808 | — | — | — | — |
| Validation KO | — | 3253 | — | — | — |
| 5′Tgmic3 (mix2) | | | | | |
| Validation KO | — | 3309 | — | — | — |
| 3′Tgmic3 (mix3) | | | | | |
| Scar Tgmic3 (mix4) | 1596 | 2525 | 226 | 226 | 226 |
| Tgmic1 (mix5) | 608 | 608 | 608 | — | — |
| Validation KO | — | — | — | 3040 | — |
| 5′Tgmic1 (mix6) | | | | | |
| Validation KO | — | — | — | 3593 | — |
| 3′Tgmic1 (mix7) | | | | | |
| Scar Tgmic1 (mix8) | 4198 | 4198 | 4198 | 3129 | 830 |

The electrophoresis of the PCR products produced with primers 5 SEQ ID NO: 61 and 6 SEQ ID NO: 62 (mix 1) allow to highlight a band with 808 base pairs for the RH strain, in accordance with the expected band size and confirming the presence of the tgmic3 gene in this strain. This band is not detected in Toxo tgmic3 KO, Toxo tgmic3 KO-2G, Toxo tgmic1-3 KO and Toxo tgmic1-3 KO-2G strains confirming gene suppression in these strains.

The electrophoresis of the PCR products made with the primers 13 SEQ ID NO: 63 and j SEQ ID NO: 25, and the primers k SEQ ID NO: 26 and 14 SEQ ID NO: 64 (mix 2 and 3) allow to highlight respectively a band at 3253 and 3537 base pairs for the Toxo tgmic3 KO strain, in accordance with the expected band size and confirming the presence of the cat-gfp selection gene SEQ ID NO: 2 in this strain instead of the tgmic3 gene SEQ ID NO: 107. This band is not detected in RH strains, Toxo tgmic3 KO-2G, Toxo tgmic1-3 KO and Toxo tgmic1-3 KO-2G confirming the absence of the cat-gfp selection gene SEQ ID NO: 2 at Tgmic 3 in these strains.

The electrophoresis of the PCR products made with the primers 9 SEQ ID NO: 65 and 10 SEQ ID NO: 66 (mix 4) allow to highlight different bands according to the expected band size. A band with 1596 base pairs for the RH strain confirms the presence of the tgmic3 gene SEQ ID NO: 107 in this strain. A tape of 2525 base pairs is highlighted for the Toxo tgmic3 KO strain confirming the presence of the CATGFP selection cassette SEQ ID NO: 6 in place of the tgmic3 gene. Finally, a band of 226 base pairs is highlighted for the strains Toxo tgmic3 KO-2G, Toxo tgmic1-3 KO and Toxo tgmic1-3 KO-2G confirming the deletion of the cat-gfp selection gene SEQ ID NO: 2 in these strains, leaving a LoxP scar SEQ ID NO: 12 in the genome.

The electrophoresis of the PCR products produced with primers 3 SEQ ID NO: 55 and 4 SEQ ID NO: 56 (mix 5) allow to highlight a band with 608 base pairs for the RH, Toxo tgmic3 KO and Toxo tgmic3 KO-2G strains, according to the expected band sizes and confirming the presence of the tgmic1 gene SEQ ID NO: 108 in these strains. These bands are not detected in Toxo tgmic1-3 KO and Toxo tgmic1-3 KO-2G strains confirming the suppression of the gene in these strains.

The electrophoresis of the PCR products made with primers 11 SEQ ID NO: 57 and j SEQ ID NO: 25, and primers k SEQ ID NO: 26 and 12 SEQ ID NO: 58 (mix 6 and 7) allow to highlight respectively a 3040 and 3593 base pairs band for the Toxo tgmic1-3 KO strain, in accordance with the expected band size and confirming the presence of the cat-gfp selection gene SEQ ID NO: 2 in this strain instead of the tgmic3 gene SEQ ID NO: 107. This band is not detected in the wild RH strains of T. gondii (ATCC reference: PRA-310), Toxo tgmic3 KO, Toxo tgmic3 KO-2G, and Toxo tgmic1-3 KO-2G confirming the absence of the cat-gfp SEQ ID NO: 2 selection gene at the Tgmic1 locus in these strains.

The electrophoresis of the PCR products made with primers 7 SEQ ID NO: 59 and 8 SEQ ID NO: 60 (mix 8) allow to highlight different bands according to the expected band size. A band of 4198 base pairs for the RH, Toxo tgmic3 KO and Toxo tgmic3 KO-2G strains confirms the presence of the tgmic1 gene SEQ ID NO: 108 in these strains. A tape of 3129 base pairs is highlighted for the Toxo tgmic1-3 KO strain confirming the presence of the CATGFP selection cassette SEQ ID NO: 6 in place of the tgmic1 gene. Finally, a band of 830 base pairs is highlighted for the Toxo tgmic1-3 KO-2G strain confirming the deletion of the cat-gfp selection gene SEQ ID NO: 2 in this strain, leaving a LoxN scar SEQ ID NO: 5 in the genome.

The “scar” PCR products (mix 4 and 8) were sequenced and the sequencing confirmed that:
the tgmic3 gene SEQ ID NO: 107 has been deleted and that the CAT-GFP selection cassette SEQ ID NO: 6 has been deleted by the action of Cre-Recombinase leaving a LoxP scar SEQ ID NO: 12 in accordance with the expected sequence (FIG. 6-C).
the tgmic1 gene SEQ ID NO: 108 has been deleted and that the CAT-GFP selection cassette SEQ ID NO: 6 has been deleted by action of the CRE-Recombinase leaving a LoxN scar SEQ ID NO: 5 in accordance with the expected sequence (FIG. 6-C).

Validation of the Toxo Tgmic1-3 KO-2G Strain by Immunofluorescence

Tachyzoites are grown 24 hours on glass lamellae covered with a monolayer of HFF cells. The infected cells were washed twice with PBS1×, and fixed with 4% formaldehyde for 30 minutes. After 3 washes in PBS1×, the infected HFF cells were permeabilized with 0.1% Triton X-100 in PBS1× for 5 min. After 3 washes with PBS 1×, a saturation step is performed with a solution of PBS 1×/SVF 10% for 30 min. The cells were then incubated with the primary antibody diluted in 2% SVF for 1 hour, washed 3 times with PBS1× and incubated with a secondary antibody diluted in 2% PBS/SVF solution for 1 hour. After 2 washes with PBS1×, the glass slides are mounted on a slide with Immu-Mount™ and observed under a fluorescence microscope.

The primary antibody Tgmic3 used is an antibody that detects the expression of the protein TgMIC3 SEQ ID NO: 121 in the parasite (rabbit anti-mic3 antibody: rnAb anti-MIC3 s3) and the secondary commercial antibody used is Alexa Fluor® 594 goat anti rabbit (Life technologies ref. A-11012).

The primary antibody Tgmic1 used is an antibody that detects the expression of the protein TgMIC1 SEQ ID NO: 122 in the parasite (mouse anti-mic 1 antibody: mAb anti-MIC1 T104F8E12) and the secondary commercial antibody used is Alexa Fluor® 594 goat anti-mouse, Life technologies ref. A-11005).

The results show that no fluorescence is detectable at the apical pole of the parasite revealing the absence of MIC1 and MIC3 proteins in tachyzoites Toxo tgmic1-3 KO-2G while fluorescence at the apical pole of the parasite of the wild strain demonstrates the expression of MIC1 and MIC3 proteins (FIG. 7).

The results show that the parasites express a green fluorescent reflecting the expression of the CATGFP protein SEQ ID NO: 120 following the completion of gene deletions (Toxo tgmic3 KO and Toxo tgmic1-3 KO) while after the action of Cre recombinase, the Toxo tgmic3 KO-2G and Toxo tgmic1-3 KO-2G strains are no longer fluorescent (CATGFP cassette removal) (FIG. 7).

### EXAMPLE 3: CONSTRUCTION OF THE TOXO TGMIC1-3 KO ROP16 KO GRA15II KI STRAIN

Material and Method

Haploidy of the Toxoplasma gondii genome during the proliferative phase allows the invalidation of a gene into a single homologous recombination.

Cultivation of Parasites

All tachyzoites of the Toxoplasma gondii strain used were produced as human fibroblasts (HFF) grown in minimal Dulbecco medium (DMEM) supplemented with 10% fetal calf serum (SVF), 2 mM glutamine, 100 U/mL penicillin and 100 U/mL streptomycin. They were collected after mechanical lysis of the host cells by 3 passages in 25G syringes.

Plasmid Construction

The plasmid pTgRop16KO-Gra15IIKI-CAT-GFP Lox2272 of SEQ ID NO: 67 (FIG. 8) contains the CAT-GFP selection cassette SEQ ID NO: 6 comprising the cat-gfp selection gene SEQ ID NO: 2, encoding the fusion protein CAT-GFP conferring chloramphenicol resistance (CAT) and green fluorescence (GFP), under the control of the promoter of α-tubulin of Toxoplasma gondii SEQ ID NO: 3 to allow gene expression in the parasite. Downstream of the selection gene SEQ ID NO: 2, the sequence 3′ UTR of the sag1 gene of Toxoplasma gondii SEQ ID NO: 4 is inserted. The objective of this sequence is to stabilize the mRNA encoding the CAT-GFP fusion protein.

The selection cassette SEQ ID NO: 6 is framed by two Lox2272 sites SEQ ID NO: 68, recognition sites specifically recognized by the Cre Recombinase and which will later be used to delete the CAT-GFP selection cassette.

Downstream of the second Lox2272 site, an expression cassette of the gra15II gene SEQ ID NO: 69 was cloned. This expression cassette consists of the gra15II promoter amplified from the genomic DNA of the ME49 strain and the gra15II gene SEQ ID NO: 70 amplified from the genomic DNA of the ME49 strain, including an HA tag in 3′ SEQ ID NO: 72 to facilitate the location and the 3′ UTR sequence of the sag1 gene of T. gondii SEQ ID NO: 4 amplified from the genomic DNA of the RH strain. This plasmid therefore allows the simultaneous realization of a rop16KO Lox2272 CATGFP Lox2272 mutant and the insertion of gra15IIHA at the rop16 locus.

The plasmid pTgROP16-KO-CAT-tdTomato SEQ ID NO: 73 contains a CAT-tdTomato cassette SEQ ID NO: 125—framed by a 5′ HR sequence of the tgrop16 gene SEQ ID NO: 99 and a 3′ HR sequence of the tgrop16 gene SEQ ID NO: 100.

Cloning was done from the plasmid pTgROP16-KO-CAT-tdTomato SEQ ID NO: 73 digested by the enzymes SphI and AgeI (6073pb). This cloning required 4 independent PCRs.

The first PCR allowed the amplification from the plasmid pCATGFP LoxP of SEQ ID NO: 38, of a CAT-GFP selection cassette SEQ ID NO: 6 comprising the promoter αTubuline, the cat-gfp selection gene and the 3′ UTR region of sag1 of T. gondii, with the addition of the Lox2272 site of SEQ ID NO: 68 with the F AgeI tub primers SEQ ID NO: 74 and 3 UTR Lox2272 R SEQ ID NO: 75 (2090pb).

The second PCR allowed amplification from the plasmid pT230 2G gra15II SEQ ID NO: 76, of the promoter gra15II SEQ ID NO: 71 with addition of the site Lox2272 of SEQ ID NO: 68 in 5′ with the primers pGra15 F Lox2272 of SEQ ID NO: 77 and P Gra15 R SEQ ID NO: 78 (1975pb).

The third PCR allowed the amplification from the plasmid pT230 2G gra15II SEQ ID NO: 76, of the gene gra15II HA with the 3′ UTR of sag1 SEQ ID NO: 79 (2092 bp) with the primers Gra15F SEQ ID NO 126 and UTR sag1 rop16 R SEQ ID NO 127.

The primers used for the construction of this plasmid are listed in Table 9 below.

TABLE 9
Primers used for the construction
of the plasmid pT230 2G Gra15II

| | |
|---|---|
| tub F AgeI | SEQ ID NO: 74: |
| | GTGATCCTGGTTGGACCGGT |
| 3 UTR 1ox2272 R | SEQ ID NO: 75: |
| | ATAACTTCGTATAAAGTATC |
| | CTATACGAAGTTATCGGTTC |
| | GACTAGAACAACTG |
| pGra15 F 1ox2272 | SEQ ID NO: 77: |
| | CTTTATACGAAGTTATGGAT |
| | CCACTAGTTGACTGCC |
| P Gra15 R | SEQ ID NO: 78: |
| | GTCACCATTGTTGAATGC |
| Gra15F | SEQ ID NO: 126: |
| | GCATTCAACAATGGTGAC |
| UTR sagl rop16 R | SEQ ID NO: 127: |
| | CGAATTTCGGGAGTTCTC |
| | GGGGGGGCAAGAATTGTG |
| Ropl6F | SEQ ID NO: 81: |
| | GAACTCCCGAAATTCGTCAA |
| Rop16R SphI | SEQ ID NO: 82: |
| | AATACACGCGACCGCATGC |

The last PCR allowed the amplification of a part of 3′Rop16 SEQ ID NO: 80 with the primers Rop16F SEQ ID NO: 81 and Rop16R SphI SEQ ID NO: 82 (570pb) on the plasmid pTgrop16 KO CAT-tdTomato SEQ ID NO: 73 as matrix.

The 4 amplified PCR fragments were directly cloned in the vector pTgrop16 KO CAT-tdTomato SEQ ID NO: 73 digested by AgeI and SphI using the Ozyme In-Fusion® kit (In-Fusion® HD Cloning Kit—Clonetech). In-Fusion® cloning technology allows one-step cloning without purification or digestion of one or more PCR products in a linearized vector. Complementary ends at the ends of adjacent fragments are added by PCR. The reaction is done according to the manufacturer's recommendations. The plasmid obtained is called pTgRop16KO-Gra15IIKI-CAT-GFP Lox2272 of SEQ ID NO: 67. The plasmid named pTgRop16KO-Gra15IIKI-CAT-GFP Lox2272 of SEQ ID NO: 67 therefore contains a CAT-GFP cassette SEQ ID NO: 6 and a gra15HAII expression cassette SEQ ID NO: 69, framed by a 5′ HR sequence of the tgrop16 gene SEQ ID NO: 99 and a 3′ HR sequence of the tgrop16 gene SEQ ID NO: 100.

Construction of the Toxo Tgmic1-3 KO Rop16 KO Gra15II KI-2G Strain

The construction of the second generation attenuated live strain, called Toxo tgmic1-3 KO rop16 KO Gra15II KI-2G, is done in 2 distinct steps:
1. Deletion by homologous recombination of the rop16 gene and its replacement by the CAT-GFP selection cassette SEQ ID NO: 6 framed by sequences Lox2272 SEQ ID NO: 38 and the cassette gra15II SEQ ID NO: 69.
   To obtain this strain, the Toxo tgmic3 KO-2G strain is electroporated with the plasmid pTgRop16KO-Gra15IIKI-CAT-GFP Lox2272 of SEQ ID NO: 67 (20 μg) linearized by PciI, according to the protocol previously described. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Toxo tgmic1-3 KO rop16 KO Gra15II KI.
   In this strain, the rop16 gene has been deleted and replaced by the CATGFP selection cassette SEQ ID NO: 6 and the expression cassette of gra15HAII SEQ ID NO: 69. Thus, the theoretical sequence at the locus of the deleted rop16 gene containing the CATGFP selection cassette and the gra15HAII expression cassette is SEQ ID NO: 112.
   In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 19.
   In this strain, the mic1 gene was deleted and the CATGFP selection cassette SEQ ID NO: 6 was excised. Thus, the theoretical sequence at the locus of the deleted mic1 gene containing a single LoxN site SEQ ID NO: 5, is SEQ ID NO: 92.
2. Deletion of the selection cassette: the obtained Toxo tgmic1-3 KO rop16 KO Gra15II KI strain is electroporated with the plasmid pT-SAG1-CRE-Recombinase SEQ ID NO: 15 (FIG. 2-C). The transiently expressed enzyme will allow the excision of the CAT-GFP selection cassette. After electroporation, the tachyzoites are deposited on a monolayer of HFF cells in culture. After 2 to 7 days of culture, the parasites are subcloned in 96-well plate on a monolayer of HFF cells and the clones of interest are identified by PCR after performing DNA genomic DNA extraction from the clones of interest. The strain obtained is called Toxo tgmic1-3 KO rop16 KO Gra15II KI-2G.
   In this strain, the rop16 gene was deleted and replaced by the expression cassette of gra15HAII SEQ ID NO: 69 and the CATGFP selection cassette of SEQ ID NO: 6 was excised. Thus, the theoretical sequence at the locus of the deleted rop16 gene containing the single site Lox2272 cassette SEQ ID NO: 68 and the expression cassette of gra15HAII is SEQ ID NO: 93.
   In this strain, the mic3 gene was deleted and the CATGFP selection cassette SEQ ID NO: 6 was excised. Thus, the theoretical sequence at the locus of the deleted mic3 gene containing a single LoxP site SEQ ID NO: 12, is SEQ ID NO: 19.
   In this strain, the mic1 gene was deleted and the CATGFP selection cassette SEQ ID NO: 6 was excised. Thus, the theoretical sequence at the locus of the deleted mic1 gene containing a single LoxN site SEQ ID NO: 5, is SEQ ID NO: 92.

Validation of the Toxo Tgmic1-3 KO Rop16 KO Gra15II KI-2G Strain by PCR

To validate the Toxo tgmic1-3 KO rop16 KO Gra15II KI 2G strain, PCR analyses are performed using genomic DNA extracted with DNAzol from a parasitic pellet composed of 10⁷ parasites. The primers used for PCR are described in FIG. 11-C and are detailed in Table 10 below. PCR products are analyzed by agarose gel electrophoresis (FIG. 9-B). Their expected and observed sizes are also described in Table 11 below.

TABLE 10
List of primers used for the validation of the
Toxo tgmic1-3 KO rop16 KO Gra15II KI 2G strain.

| | | Primer | | | Primer | |
|---|---|---|---|---|---|---|
| | | F | Sequence | | R | Sequence |
| Tgrop16 | Rg 1 | AGD rop | SEQ ID NO: 83 | Rg 2 | AGD rop | SEQ ID NO: 84 |
| (mix1) | | 16CDS F | GTTTGAGGAAGCGC | | 16CDS R | TGCTGTGATTTCGC |
| | | | AAAAAG | | | AAGTTC |
| Upstream | Rg 3 | Rop 16KO | SEQ ID NO: 85 | Rg 4 | Amont | SEQ ID NO: 86 |
| KO | | valid | ACCCCCTGACCCCT | | ropl6R | TCGTCGTGGTATTC |
| validation | | 5F1 | TGTCTG | | | ACTCCA |
| (mix2) | | | | | | |
| Downstream | Rg 5 | Gra15 | SEQ ID NO: 87 | Rg 6 | Aval | SEQ ID NO: 88 |
| KO | | qPCR 2F | CACGTACACAACCC | | ropl6R | AAAACGAATGCGAA |
| validation | | | ATCTCG | | | GGAAGA |
| (mix3) | | | | | | |
| Scar | Rg 7 | cicropl6 | SEQ ID NO: 89 | Rg 8 | cicrop16 | SEQ ID NO: 90 |
| rop16KO | | loxF | CAGTACCAGCCACG | | loxGra R | CAAGCAATCTGTGG |
| gra15 | | | TTAGCA | | | CAAAAA |
| LoxP | | | | | | |
| gra15 | | | | | | |
| (mix4) | | | | | | |
| Gra15I/II | | Gra15I/ | SEQ ID NO: 109 | | Gra15I/ | SEQ ID NO: 110 |
| (mix5) | | II F | GTGCAAAGCCAACT | | II F | GGACCTGGATCAGA |
| | | | TCCACT | | | GATCGT |

TABLE 11
Expected amplicon size (in base pairs) of the different
PCRs validating the construction of the Toxo tgmic1-3
KO rop16 KO Gra15II KI 2G strain

| | Toxo | Toxo tgmic1-3 | Toxo tgmic1-3 |
|---|---|---|---|
| | tgmic1-3 | KO rop16 KO | KO rop16 KO |
| | KO - 2G | Gra15II KI | Gra15II KI -2G |
| Tgrop16 (mix1) | 1682 | — | — |
| Validation KO | — | 2759 | — |
| 5′ Tgrop16 (mix2) | | | |
| Validation KO | — | 2870 | 2870 |
| 3′ Tgrop16 (mix3) | | | |
| Scar rop16KO | — | 2550 | 321 |
| gra15LoxP (mix4) | | | |
| Gra15I/II (mix5) | 560 | 560 and 308 | 560 and 308 |

The electrophoresis of the PCR products produced with the primers rg1 SEQ ID NO: 83 and rg2 SEQ ID NO: 84 (mix 1) allow to highlight a band with 1682 base pairs for the Toxo tgmic1-3 KO-2G strain, according to the expected band size and confirming the presence of the tgrop16 gene in this strain. This band is not detected in Toxo tgmic1-3 KO rop16 KO Gra15II KI and Toxo tgmic1-3 KO rop16 KO Gra15II KI-2G strains confirming gene suppression in these strains.

The electrophoresis of the PCR products produced with the primers rg3 SEQ ID NO: 85 and rg4 SEQ ID NO: 86 (mix 2) make it possible to highlight a band with 2759 base pairs for the Toxo tgmic1-3 KO rop16 KO Gra15II KI strain, in accordance with the size of the expected bands and confirming the suppression of the tgrop16 gene in this strain and the insertion of the CATGFP and gra15II selection genes instead of the tgrop16 gene. This band is not detected in Toxo tgmic1-3 KO-2G and Toxo tgmic1-3 KO rop16 KO Gra15II KI-2G strains. The electrophoresis of the PCR products made with the primers rg5 SEQ ID NO: 87 and rg6 SEQ ID NO: 88 (mix 3) allow to highlight a band with 2870 base pairs for the strains Toxo tgmic1-3 KO rop16 KO Gra15II KI and Toxo tgmic1-3 KO rop16 KO Gra15II KI-2G, according to the size of the expected bands and confirming the suppression of the tgrop16 gene in this strain and the insertion of the gra15II gene instead of the tgrop16 gene. This band is not detected in the Toxo tgmic1-3 KO-2G strain.

The electrophoresis of the PCR products produced with the primers rg7 SEQ ID NO: 89 and rg8 SEQ ID NO: 90 (mix 4) allow to highlight a band of 2550 base pairs for the Toxo tgmic1-3 KO rop16 KO Gra15II KI strain confirming the presence of the CATGFP selection cassette SEQ ID NO: 6 in place of the tgrop16 gene. Finally, a band of 321 base pairs is identified for the Toxo tgmic1-3 KO rop16 KO Gra15II KI-2G strain confirming the deletion of the cat-gfp selection gene SEQ ID NO: 2 in this strain, leaving a Lox2272 of SEQ ID NO: 68 scar in the genome. No bands are not detected in the Toxo tgmic1-3 KO-2G strain.

The “scar” PCR product obtained for the Toxo tgmic1-3 KO rop16 KO Gra15II KI-2G strain (321 base pairs) was sequenced and the sequencing confirmed that:
the tgrop16 gene has been deleted and that the CAT-GFP selection cassette SEQ ID NO: 6 has been deleted by the action of Cre-Recombinase leaving a Lox2272 scar SEQ ID NO: 68 in accordance with the expected sequence (FIG. 9-C).

The electrophoresis of the PCR products produced with the primers SEQ ID NO: 109 and SEQ ID NO: 110 (mix 5) make it possible to highlight a band with 560 base pairs for the Toxo tgmic1-3 KO-2G strains, in accordance with the expected band size and confirming the presence of the tggra15 gene in this strain. An additional band of 308 base pairs is detected in Toxo tgmic1-3 KO rop16 KO Gra15II KI and Toxo tgmic1-3 KO rop16 KO Gra15II KI-2G strains confirming the presence of the gra15HAII gene in these strains.

Validation of the Toxo Tgmic1-3 KO Rop16 KO Gra15II KI-2G Strain by Immunofluorescence

Tachyzoites are grown 24 hours on glass lamellae covered with a monolayer of HFF cells. The infected cells were washed twice with PBS1×, and fixed with 4% formaldehyde for 30 minutes. After 3 washes in PBS1×, the infected HFF cells were permeabilized with 0.1% Triton X-100 in PBS1× for 5 min. After 3 washes with PBS 1×, a saturation step is performed with a solution of PBS 1×/SVF 10% for 30 min. The cells were then incubated with the primary antibody diluted in 2% SVF for 1 hour, washed 3 times with PBS1× and incubated with a secondary antibody diluted in 2% PBS/SVF solution for 1 hour. After 2 washes with PBS1×, the glass slides are mounted on a slide with Immu-Mount™ and observed under a fluorescence microscope.

The primary antibody used is the anti-HA mouse antibody which detects the expression of the protein GRA15II-HA SEQ ID NO: 123 in the parasite. The secondary antibody is the commercial secondary antibody: Alexa Fluor® 594 anti rabbit goat (Life Technologies A-11012). The antibodies are diluted 1/1000 times in 2% SVF PBS.

For the wild strain Toxo tgmic1-3 KO 2G, no red fluorescence is observed at the apical pole of the parasite, revealing the absence of the protein GRA15II-HA. On the contrary, for the Toxo tgmic1-3 KO rop16 KO gra15II KI strain, a red fluorescence is observed showing the protein GRA15II-HA. The Toxo tgmic1-3 KO rop16 KO gra15II KI strain expresses a green fluorescent reflecting the expression of the CATGFP protein SEQ ID NO: 120 whereas after the action of the Cre recombinase, the Toxo tgmic1-3 KO rop16 KO gra15II KI Lox2272 strain is no longer fluorescent (FIG. 10).

### EXAMPLE 4: CONSTRUCTION OF RECOMBINANT STRAINS EXPRESSING THE M2eGPI PROTEIN AFTER TARGETED INTEGRATION

The objective of this experiment is to evaluate whether the strains Toxo tgmic1-3 KO 2G and Neo ncmic1-3 KO 2G can express heterologous antigens.

Cultivation of Parasites

All tachyzoites of the Toxoplasma gondii strain used were produced as human fibroblasts (HFF) grown in minimal Dulbecco medium (DMEM) supplemented with 10% fetal calf serum (SVF), 2 mM glutamine, 100 U/mL penicillin and 100 U/mL streptomycin. They were collected after mechanical lysis of the host cells by 3 passages in 25G syringes.

Plasmid Construction

Plasmid pUC 4G2 ICreI

The plasmid pUC 4G2 ICreI SEQ ID NO: 156 was constructed to allow the integration of a heterologous transgene into the strains Toxo tgmic1-3 KO 2G, Neo ncmic1-3 KO 2G and Toxo tgmic1-3 KO rop16 KO gra15II KI.

The plasmid is notably composed of:
1—a selection cassette dhfr*-ty-tk SEQ ID NO: 229 (Donald & Roos, Proc Natl Acad Sci U S S A. 1993 Dec. 15; 90(24):11703-7; Scahill et al., (Mol Biochem Parasitol. 2008 January; 157(1):73-82. Epub 2007 Oct. 6.)), placed under the dependence of the dhfr* promoter SEQ ID NO: 230 of T. gondii and downstream of the 3′ UTR sequence of the dhfr* gene SEQ ID NO: 231 of Toxoplasma gondii. This selection cassette encodes the fusion protein DHFR*TYTK SEQ ID NO: 207 and allows positive selection by pyrimethamine and negative selection by ganciclovir.
2—an expression cassette consisting of the promoter of α-Tub8 SEQ ID NO: 128 (Soldati et al., Mol Cell Biol. 1995 January; 15(1):87-93—fusion of part of the promoter of Tgsag1 and Tgtub), an MCS multiple cloning site to allow future integration of heterologous transgene and the 3′ UTR sequence of the tgsag1 gene SEQ ID NO: 4 which should stabilize the mRNA of the heterologous gene.

The dhfr*-ty-tk selection cassette was made from 3 PCR fragments obtained with the primers in Table 20:
The amplification of the first PCR fragment SEQ ID NO: 129 was performed on the plasmid pUC18DHFR* SEQ ID NO: 130 (Donald & Roos 1993, same reference as above) with the Dhtk1 primers SEQ ID NO: 131 and Dhtk2 SEQ ID NO: 132 (674pb) and allows the amplification of the end of the coding sequence of DHFR* SEQ ID NO: 133 and to add the sequence coding the TY in 3′ SEQ ID NO: 134.
The second PCR was performed on a synthesized sequence of the Human herpesvirus 1 (TK) Thymidine Kinase gene SEQ ID NO: 135 with Dhtk3 SEQ ID NO: 136 and Dhtk4 SEQ ID NO: 137 (1161pb) primers and allows the amplification of the Thymidine Kinase coding sequence with the addition of the TY coding sequence in 5′ SEQ ID NO: 138.
The amplification of the third PCR fragment was performed on pUC18DHFR* SEQ ID NO: 130 with Dhtk5 primers SEQ ID NO: 139 and Dhtk6 SEQ ID NO: 140 (363pb) and allows the amplification of the end of the coding sequence of Thymidine Kinase and the 3′ UTR of DHFR* SEQ ID NO: 141.

The second and third PCR fragments are fused by overlapping PCR with the primers Dhtk3 SEQ ID NO: 136 and Dhtk6 SEQ ID NO: 140 (1501pb) to give the sequence SEQ ID NO: 191. Finally, the first fragment SEQ ID NO: 129 digested by BamHI and BglII (646pb) and the fusion of the other two PCR SEQ ID NO: 191 digested by BamHI and XbaI (1474pb) are cloned in the plasmid pUC18DHFR* SEQ ID NO: 130 digested by BglII and XbaI (5258pb). The plasmid obtained is called pUC18DHFR*TYTK SEQ ID NO: 142.

TABLE 20
List of primers used

| | | | | | |
|---|---|---|---|---|---|
| Dhtk | AF | SEQ ID NO: 131 | Dhtk | AF | SEQ ID NO: 132 |
| 1 | DHFRi | CTGAGAAGGGCGTG | 2 | DHFR | GTCAAGTGGATCCT |
| | nt | AAGATC | | TY R | GGTTAGTATGGACC |
| | Bgl | | | | TCGACAGCCATCTC |
| | F | | | | CATCTGGATTCG |
| Dhtk | TY | SEQ ID NO: 136 | Dhtk | HSVT | SEQ ID NO: 137 |
| 3 | HSVT | GAGGTCCATACTAA | 4 | K stop | TTAGTTAGCCTCCC |
| | K F | CCAGGATCCACTTG | | TAA R | CCATCTCCC |
| | | ACATGGCTTCGTAC | | | |
| | | CCCTGCCATCA | | | |
| Dhtk | AFHS | SEQ ID NO: 139 | Dhtk | AF | SEQ ID NO: 140 |
| 5 | VTK3 | GGGAGATGGGGGAG | 6 | 3UTR | TTTTTCTAGAATCC |
| | UTRD | GCTAACTAACGGAA | | DHFR | TGCAAGTGCATAGA |
| | HFRF | ATACAGAAGCTGCC | | XbaR | AGGAA |
| | | CGTCTCT | | | |

The expression cassette is composed of the promoter of α-Tub8 SEQ ID NO: 128 (Soldati et al, 1995, same reference as above), a multiple cloning site to allow future integration of heterologous transgene and the 3′ UTR sequence of the sag1 gene SEQ ID NO: 4 which should stabilize the mRNA of the heterologous gene. The promoter part of α-Tub8 SEQ ID NO: 128 was obtained by PCR with the primers K7mcs1 SEQ ID NO: 143 and K7mcs2 SEQ ID NO: 144 (530pb) on the pTUB8TY TAIL SEQ ID NO: 145 (Meissner et al., J Cell Sci. 2002; 115:563-574). The 3′ UTR sequence of the sag1 gene SEQ ID NO: 4 was obtained by PCR with the primers K7mcs3 SEQ ID NO: 146 and K7mcs4 SEQ ID NO: 147 (395 bp). Then the two PCR fragments were fused by overlapping PCR with the primers K7mcs1 SEQ ID NO: 143 and K7mcs4 SEQ ID NO: 147 (914pb) to give the sequence SEQ ID NO: 192. The overlapping PCR SEQ ID NO: 192 digested by KpnI and SacI (902pb) was cloned in pUC18 (commercial plasmid) KpnI SacI (2682pb). The primers used for PCRs are detailed in Table 12 below. The plasmid obtained is pUC 18-Tub8MCS 3UTR SEQ ID NO: 148.

TABLE 12
List of primers used for
the construction of the
expression cassette

| | Primer | | | Primer | |
|---|---|---|---|---|---|
| | F | Sequence | | R | Sequence |
| K7mcs1 | pTUB8 | SEQ ID | K7mcs2 | Tub 8 | SEQ ID |
| | MCS 4 | NO: 143 | | Ter R | NO: 144 |
| | F | AAAGGTA | | | AACTTAAG |
| | | CCTCTAG | | | AATTTTGT |
| | | AAGTATA | | | TTAAACAG |
| | | CTATTCG | | | GG |
| | | AAAAACT | | | |
| | | AGTATCG | | | |
| | | ATAAGCT | | | |
| | | GAACAC | | | |
| K7mcs3 | MCS | SEQ ID | K7mcs4 | MCS | SEQ ID |
| | 3UTR | NO: 146 | | 3UT R | NO: 147 |
| | TER F | TTTAAAC | | TER R | TTTGAGCT |
| | | AAAATTC | | | CTCTAGAA |
| | | TTAAGTT | | | TTTAAATC |
| | | GCGGC | | | CTAGG |

The expression cassette Tub8MCS 3UTR SEQ ID NO: 149 obtained by digesting pUC 18-Tub8MCS 3UTR SEQ ID NO: 148 with the enzyme XbaI (890pb) was then cloned in the plasmid pUC18 DHFR*TYTK SEQ ID NO: 142 digested by XbaI and dephosphorylated (7378pb). The plasmid obtained has its Tub8MCS 3UTR expression cassette transcribed in the opposite direction to the DHFR*TYTK cassette. The plasmid obtained is called pUC4G2 SEQ ID NO: 150.

The PCR fragment SEQ ID NO: 193 amplified with the primers IcreI1 SEQ ID NO: 151 and IcreI2 SEQ ID NO: 152 on the pTsag1IcreI αβγ SEQ ID NO: 153 (142pb) and the PCR fragment SEQ ID NO: 194 amplified with the primers IcreI3 SEQ ID NO: 154 and IcreI4 SEQ ID NO: 155 on pUC4G2 SEQ ID NO: 150 (153pb) allow the addition of the sequence αβ IcreI SEQ ID NO: 157. The two PCR fragments SEQ ID NO: 193 and SEQ ID NO: 194 were directly cloned in the plasmid pUC4G2 SEQ ID NO: 150 digested by the restriction enzymes Pml I and KasI (7990pb) with the Ozyme In-Fusion technique.

Then the fragment of PCR SEQ ID NO: 195 amplified with the primers IcreI5 SEQ ID NO: 159 and IcreI6 SEQ ID NO: 160 on the pTIcreI100 SEQ ID NO: 158 (546pb) and the fragment of PCR SEQ ID NO: 196 amplified with the primers IcreI7 SEQ ID NO: 161 and IcreI8 SEQ ID NO: 162 on pTsag1IcreI αβγ SEQ ID NO: 153 (133pb) allow the addition of the sequence IcreI βγ SEQ ID NO: 163 in the plasmid previously described digested by the restriction enzymes NsiI and AvrII (7734pb). The plasmid obtained is called pUc4G2 IcreI SEQ ID NO: 164. The primers used for PCRs are detailed in Table 21 below.

TABLE 21
List of primers used

| | | | | | |
|---|---|---|---|---|---|
| Icrei | PCR | SEQ ID NO: 151 | Icrei | oz4g | SEQ ID NO: 152 |
| 1 | oz4g | AATACCGCATCAGGCGCCTAGAA | 2 | mn1Ic | AAAACGTCGTGAGA |
| | mn1 | CCTGTTAAAAATAT | | reIr | CAGTTTGGTGAGAT |
| | F | | | | ATCTTCAAAAGATA |
| | | | | | TATAGC |
| Icrei | oz4g | SEQ ID NO: 154 | Icrei | oz4g | SEQ ID NO: 155 |
| 3 | mn2 | GTCTCACGACGTTTTGAACCCAG [symbol] | 4 | mn2 R | CGGATATGGTTACA |
| | IcreIF | [symbol] | | | CGTG |
| Icrei | oz4g | SEQ ID NO: 159 | Icrei | oz4g3 | SEQ ID NO: 160 |
| 5 | 3F | CGCCTCCGTCCCATGCAT | 6 | cre R | CCAAACTGTCTCAC |
| | | | | | GACGTT |
| Icrei | oz4g4 | SEQ ID NO: 161 | Icrei | oz4g4 | SEQ ID NO: 162 |
| 7 | FIcreI | CGTGAGACAGTTTGGCTAAAAATT | 8 | Ravr | GAGGCGCGCCAACC |
| | | TATTTTAAATAATCTTAT | | II | TAGGAGGGCCCGGG |
| | | | | | CGCCATGGC |

Plasmid pUC 4G2 IcreI M2eGPI SEQ ID NO: 164

This plasmid will allow the production of a fusion protein sag1 M2eGPI SEQ ID NO: 165, comprising the protein SAG1 of Toxoplasma gondii SEQ ID NO: 166, a 5 M2e repetition (Nter fragment of Influenza virus protein M2) spaced by linkers (Lee et al, 2015, PLoS ONE 10(9): e0137822. doi:10.1371/day.pone.0137822) SEQ ID NO: 167 and Cter a GPI anchoring sequence of the SAG1 protein of Toxoplasma gondii SEQ ID NO: 169. The amplification of 3 PCR fragments was necessary for this construction. The amplification of the first PCR fragment SEQ ID NO: 197 was performed on the genomic DNA of Toxoplasma gondii of the coding sequence of sag1 SEQ ID NO: 170 with the primers da SEQ ID NO: 171 and db SEQ ID NO: 172 (906pb). The second PCR SEQ ID NO: 198 was performed on a synthesized sequence comprising a repetition of 5 M2e SEQ ID NO: 173 (Lee et al) with primers dc SEQ ID NO: 174 and dd SEQ ID NO: 175 (588pb). This M2e sequence has been optimized for expression in Toxoplasma gondii. The amplification of the third PCR fragment SEQ ID NO: 199 was performed on the genomic DNA of Toxoplasma gondii of the coding sequence of sag1 (GPI anchor part) SEQ ID NO: 176 with the primers of SEQ ID NO: 177 and df SEQ ID NO: 178 (98pb). The primers used for PCRs are detailed in Table 13 below.

TABLE 13
List of primers used for the construction
of plasmids pUC 4G2 IcreI M2eGPI

| | Primer | | | Primer | |
|---|---|---|---|---|---|
| | F | Sequence | | R | Sequence |
| da | Ozsa | SEQ ID NO: 171 | db | Sag1 | SEQ ID NO: 172 |
| | g14G F | CTTGAATTCCCTGT | | M2e R | TCAGCAAGGACCTA |
| | | TTAAACGACAAAAt | | | GGTGCAGCCCCGGC |
| | | gtttccgaaggcag | | | AA |
| | | tg | | | |
| dc | M2e F | SEQ ID NO: 174 | dd | OzM2e | SEQ ID NO: 175 |
| | | CCTAGGTCCTTGCT | | GPI R | GGCTGTTCCCGCAG |
| | | GACTGA | | | CCGTAGAATCGAGA |
| | | | | | CCGAGGA |
| de | oZ | SEQ ID NO: 177 | df | oZ | SEQ ID NO: 178 |
| | GPI F | GCTGCGGGAACAGC | | GPI R | ACCATGGAAGCGGC |
| | | CAGTCA | | | CGCTTACGCGACAC |
| | | | | | AAGCTGCGAT |

The PCR fragments were directly cloned in the plasmid p4G2 ICreI SEQ ID NO: 156 digested by the enzymes PmeI and NotI (8344pb) with the Ozyme In-Fusion technique.

Plasmid pUC 4G2 IcreI M2eGPI LoxP of SEQ ID NO: 179

The LoxP site of SEQ ID NO: 12 was introduced by PCR into the plasmid PUC 4G2 IcreI M2eGPI SEQ ID NO: 164 digested PciI and PmeI (8901 bp). The PCR fragment SEQ ID NO: 200 obtained with the primers aa SEQ ID NO: 180 and ab SEQ ID NO: 181 (438pb) and the PCR fragment SEQ ID NO: 201 obtained with the primers ac SEQ ID NO: 182 and ad SEQ ID NO: 183 (534pb) were cloned with the Ozyme In-Fusion technique in the plasmid PUC 4G2 IcreI M2eGPI SEQ ID NO: 164 digested PciI and PmeI. The primers used for PCRs are detailed in Table 14 below.

TABLE 14
List of primers used for the
introduction of the LoxP site

| | Primer | | | Primer | |
|---|---|---|---|---|---|
| | F | Sequence | | R | Sequence |
| aa | pUC4G | SEQ ID NO: 180 | ab | Puc4G | SEQ ID NO: 181 |
| | PciI F | GCTGGCCTTTTGCT | | loxP R | TATAGCATACATTA |
| | | CACATGT | | | TACGAAGTTATTAG |
| | | | | | TATACTTCTAGAGG |
| | | | | | ATCC |
| ac | Puc4G | SEQ ID NO: 182 | ad | pUC4G | SEQ ID NO: 183 |
| | loxP F | TATAATGTATGCTA | | PmeI R | GAAACATTTTGTCG |
| | | TACGAAGTTATAAA | | | TTTAAAC |
| | | CTAGTATCGATAAG | | | |
| | | CTTG | | | |

Plasmid pUC 4G2 IcreI M2eGPI LoxN of SEQ ID NO: 184

The LoxN site SEQ ID NO: 5 was introduced by PCR into the plasmid PUC 4G2 IcreI M2eGPI SEQ ID NO: 164 digested PciI and PmeI (8901 bp). The PCR fragment SEQ ID NO: 202 obtained with the primers aa SEQ ID NO: 180 and bb SEQ ID NO: 181 (438pb) and the PCR fragment SEQ ID NO: 203 obtained with the primers be SEQ ID NO: 182 and ad SEQ ID NO: 183 (534pb) were cloned with the Ozyme In-Fusion technique in the plasmid PUC 4G2 IcreI M2eGPI SEQ ID NO: 164 digested PciI and PmeI. The primers used for PCRs are detailed in Table 15 below.

TABLE 15
List of primers used for the
introduction of the LoxN site

| | Primer | | | Primer | |
|---|---|---|---|---|---|
| | F | Sequence | | R | Sequence |
| aa | pUC4G | SEQ ID NO: 180 | bb | Puc4G | SEQ ID NO: 185 |
| | PciI | GCTGGCCTTTTGCT | | loxN R | TATAAGGTATACTA |
| | F | CACATGT | | | TACGAAGTTATTAG |
| | | | | | TATACTTCTAGAGG |
| | | | | | ATCC |
| bc | Puc4G | SEQ ID NO: 186 | ad | pUC4G | SEQ ID NO: 183 |
| | loxN | TATAGTATACCTTA | | PmeI R | GAAACATTTTGTCG |
| | F | TACGAAGTTATAAA | | | TTTAAAC |
| | | CTAGTATCGATAAG | | | |
| | | CTTG | | | |

Construction of Toxo Tgmic1-3 KO-2G M2eGPI Strains Targeted or Random Integration. Targeted Integration into the LoxP Site

The ToxoKO mic1-3KO 2G strain is electroporated with 20 μg of purified circular plasmids pUC 4G2 IcreI M2eGPI LoxP of SEQ ID NO: 179 and pTsag1 Cre recombinase SEQ ID NO: 15 according to the previously described protocol. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Toxo tgmic1-3 KO-2G M2eGPI LoxP.

Targeted Integration into the LoxN Site

The ToxoKO mic1-3KO 2G strain is electroporated with 20 μg of purified circular plasmids pUC 4G2 IcreI M2eGPI LoxN of SEQ ID NO: 184 and pTsag1 Cre recombinase SEQ ID NO: 15 according to the protocol previously described. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Toxo tgmic1-3 KO-2G M2eGPI LoxN.

Random Integration

The ToxoKO mic1-3KO 2G strain is electroporated with 20 μg of the purified linearized plasmid pUC 4G2 IcreI M2eGPI LoxP of SEQ ID NO: 179 or pUC 4G2 IcreI M2eGPI LoxN of SEQ ID NO: 184 according to the previously described protocol. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are analyzed for the expression of the fusion protein SAG1 M2eGPI SEQ ID NO: 165 (analysis on the total population).

Validation Toxo Tgmic1-3 KO-2G M2eGPI Targeted Integration by PCR Targeted Integration into the LoxP Site

Two PCRs are performed to validate clones that have selectively integrated the plasmid. To validate strains that have integrated the plasmids pUC 4G2 IcreI M2eGPI LoxP SEQ ID NO: 179, PCR analyses are performed from genomic DNA extracted with DNAzol from a parasitic pellet composed of 10⁷ parasites. The primers used for PCRs are detailed in Table 16 below. PCR products are analyzed by agarose gel electrophoresis. The clones that integrated the plasmid were validated by PCR with the primers ea SEQ ID NO: 187 and eb SEQ ID NO: 188 by obtaining the fragment SEQ ID NO: 204 (1719pb).

The second PCR validates the location of the plasmid insertion at the Tgmic3 LoxP locus. The clones having integrated one of the plasmids were validated by PCR with the primers ec SEQ ID NO: 189 and eb SEQ ID NO: 188 by obtaining the fragment SEQ ID NO: 205 (2704 bp).

TABLE 16
List of primers used for clone validation

| | Primer | | | Primer | |
|---|---|---|---|---|---|
| | F | Sequence | | R | Sequence |
| ea | seq | SEQ ID NO: 187 | eb | seq | SEQ ID NO: 188 |
| | TUB | AACCCGCGCAGAAG | | 3utr | ATGGGGCACATGCT |
| | MCS F | ACATCC | | MCS R | GCACGAA |
| ec | CN3 | SEQ ID NO: 189 | eb | seq | SEQ ID NO: 188 |
| | | AGAGGTTGACACCG | | 3utr | ATGGGGCACATGCT |
| | | ACAAAG | | MCS R | GCACGAA |

Targeted Integration into the LoxN Site

Two PCRs are performed to validate clones that have selectively integrated one of the plasmids. To validate strains that have integrated the plasmid pUC 4G2 IcreI M2eGPI LoxN of SEQ ID NO: 184, PCR analyses are performed from genomic DNA extracted with DNAzol from a parasitic pellet composed of 10⁷ parasites. The primers used for PCRs are detailed in Table 17 below. PCR products are analyzed by agarose gel electrophoresis. The clones having integrated one of the plasmids were validated by PCR with the primers ea SEQ ID NO: 187 and eb SEQ ID NO: 188 (1719pb) by obtaining the fragment SEQ ID NO: 204.

The second PCR is used to validate the location of the plasmid insertion at the Tgmic1 LoxN locus. The clones having integrated one of the plasmids were validated by PCR with the primers ed SEQ ID NO: 55 and eb SEQ ID NO: 54 (2366pb) by obtaining the fragment SEQ ID NO: 206.

TABLE 17
List of primers used for clone validation

| | Primer | | | Primer | |
|---|---|---|---|---|---|
| | F | Sequence | | R | Sequence |
| ed | Tg | SEQ ID NO: 190 | eb | seq | SEQ ID NO: 188 |
| | mic1 | CCCGTCTAGCAAGA | | 3utr | ATGGGGCACATGCT |
| | loxN | CCTCAA | | MCS R | GCACGAA |

Analysis by Flow Cytometry.

The expression level of M2eGPI SEQ ID NO: 165 was analyzed for different populations of recombinant parasites following specific labelling with an anti-M2 antibody (anti-influenza A virus M2 Protein antibody ab5416—Abcam). The expression of the M2eGPI transgene is similar or slightly higher for clones whose transgene is inserted in a targeted manner (located at the LoxP and LoxN scars at the mic1 or mic3 locus) than for populations whose transgene is inserted in a random manner. This result shows that the Toxo tgmic1-3 KO 2G strain is an expression vector optimized for transgene expression.

### EXAMPLE 5: CONSTRUCTION OF RECOMBINANT STRAINS OF TOXO TGMIC1-3 KO EXPRESSING THE CAT-GFP PROTEIN AFTER TARGETED OR RANDOM INTEGRATION OF THE CAT-GFP TRANSGENE

The objective of this experiment is to study the level of expression of the cat-gpf gene after random or targeted integration at the LoxP or LoxN site of the transgene.

Cultivation of Parasites

All tachyzoites of the Toxoplasma gondii strain used were produced as human fibroblasts (HFF) grown in minimal Dulbecco medium (DMEM) supplemented with 10% fetal calf serum (SVF), 2 mM glutamine, 100 U/mL penicillin and 100 U/mL streptomycin. They were collected after mechanical lysis of the host cells by 3 passages in 25G syringes.

Plasmid Construction

Plasmid pUC 4G2 IcreI CAT-GFP LoxP LoxP of SEQ ID NO: 221

The CATGFP fragment SEQ ID NO: 222 was amplified by PCR with the primers ca SEQ ID NO: 223 and cb SEQ ID NO: 224 (1488 bp) on the pCATGFP SEQ ID NO: 9. This PCR fragment was cloned with the In-Fusion technique of Ozyme in the plasmid pUC 4G2 IcreI M2eGPI LoxP of SEQ ID NO: 179 was digested by PmeI and NotI (8372pb-replacing M2eGPI by CATGFP). The primers used for PCRs are detailed in Table 18 below.

TABLE 18
List of primers used for the construction
of plasmids pUC 4G2 IcreI CAT-GFP

| | Primer | Sequence | | Primer | |
|---|---|---|---|---|---|
| | F | | | R | Sequence |
| ca | Oz | SEQ ID NO: 223 | cb | Oz | SEQ ID NO: 224 |
| | CAT | TTGAATTCCCTGTT | | CAT | ACCATGGAAGCGGC |
| | GFP | TCGACAAAATGCAT | | GFP | CGCTTAATCGAGCG |
| | 4G F | GAGAA | | 4G R | GGTCCTGG |

Plasmid pUC 4G2 IcreI CAT-GFP LoxN of SEQ ID NO: 225

The CATGFP fragment SEQ ID NO: 222 was amplified by PCR with the primers ca SEQ ID NO: 223 and cb SEQ ID NO: 224 (1488 bp) on the pCATGFP SEQ ID NO: 9. This PCR fragment was cloned with the In-Fusion technique of Ozyme in the plasmid pUC 4G2 IcreI M2eGPI LoxN of SEQ ID NO: 184 was digested by PmeI and NotI (8372pb-replacing M2eGPI by CATGFP). The primers used for PCRs are detailed in Table 18 above.

Construction of Toxo Tgmic1-3 KO-2G CATGFP Strains Targeted or Random Integration.

Targeted Integration into the LoxP Site

The ToxoKO mic1-3KO 2G strain is electroporated with 20 μg of purified circular plasmids pUC 4G2 IcreI CATGFP LoxP of SEQ ID NO: 221 and pTsag1 Cre recombinase SEQ ID NO: 15 according to the protocol previously described. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Toxo tgmic1-3 KO-2G CATGFP LoxP.

Targeted Integration into the LoxN Site

The ToxoKO mic1-3KO 2G strain is electroporated with 20 μg of purified circular plasmids pUC 4G2 IcreI CATGFP LoxN of SEQ ID NO: 225 and pTsag1 Cre recombinase SEQ ID NO: 15 according to the protocol previously described. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are subcloned in 96-well plate on a monolayer of HFF cells and the clones of interest are identified by PCR after performing genomic DNA extraction from the clones of interest. The strain obtained is called Toxo tgmic1-3 KO-2G CATGFP LoxN.

Random Integration

The ToxoKO mic1-3KO 2G strain is electroporated with 20 μg of the purified linearized plasmid pUC 4G2 IcreI CATGFP LoxP of SEQ ID NO: 221 or pUC 4G2 IcreI CATGFP LoxN of SEQ ID NO: 225 according to the previously described protocol. After electroporation, the tachyzoites are deposited on a monolayer of HFF cells in culture. For mutant selection, the culture medium is replaced and supplemented by the selection agent (chloramphenicol 20 μM) 24 hours after electroporation. 10 to 15 days after selection, the parasites are analyzed for the expression of the CATGFP protein SEQ ID NO: 120 (analysis on the total population).

Validation Toxo Tgmic1-3 KO-2G CATGFP Targeted Integration by PCR

Targeted Integration into the LoxP Site

Two PCRs are performed to validate clones that have selectively integrated the plasmid. To validate strains that have integrated the plasmids pUC 4G2 IcreI CATGFP LoxP SEQ ID NO: 221, PCR analyses are performed from genomic DNA extracted with DNAzol from a parasitic pellet composed of 10⁷ parasites. The primers used for PCRs are detailed in Table 16 above. PCR products are analyzed by agarose gel electrophoresis. The clones that integrated the plasmid were validated by PCR with the primers ea SEQ ID NO: 187 and eb SEQ ID NO: 188 and obtaining a fragment SEQ ID NO: 226 (1647pb).

The second PCR validates the location of the plasmid insertion at the Tgmic3 LoxP locus. The clones having integrated one of the plasmids were validated by PCR with the primers ec SEQ ID NO: 189 and eb SEQ ID NO: 188 and obtaining a fragment SEQ ID NO: 227 (2600 bp). The primers used for PCRs are detailed in Table 16.

Targeted Integration into the LoxN Site

Two PCRs are performed to validate clones that have selectively integrated the plasmid. To validate strains that have integrated the plasmid pUC 4G2 IcreI CATGFP LoxN SEQ ID NO: 225, PCR analyses are performed from genomic DNA extracted with DNAzol from a parasitic pellet composed of 10⁷ parasites. The primers used for PCRs are detailed in Table 17 above. PCR products are analyzed by agarose gel electrophoresis. The clones having integrated one of the plasmids were validated by PCR with the primers ea SEQ ID NO: 187 and eb SEQ ID NO: 188 and obtaining a fragment SEQ ID NO: 226 (1647pb). The primers used for PCRs are detailed in Table 16.

The second PCR is used to validate the location of the plasmid insertion at the Tgmic1 LoxN locus. The clones having integrated one of the plasmids were validated by PCR with the primers ed SEQ ID NO: 190 and eb SEQ ID NO: 188 and obtaining a fragment SEQ ID NO: 228 (2294 bp). The primers used for PCRs are detailed in Table 17.

Flow Cytometry Analysis

The expression level of CAT-GFP SEQ ID NO: 120 was analyzed in the different populations of recombinant parasites (Table 19). The expression of the CATGFP transgene is systematically slightly higher for clones whose transgene is inserted in a targeted manner (located at the LoxP and LoxN scars at the mic1 or mic3 locus) than for populations whose transgene is inserted in a random manner.

TABLE 19
Geometric mean fluorescence intensity of recombinant CATGFP strains.

| | Geometric mean fluorescence | | Geometric mean fluorescence |
|---|---|---|---|
| Parasite | intensity (GFP) | Parasite | intensity (GFP) |
| ToxoKO 2G | 912 | ToxoKO 2G | 912 |
| ToxoKO 2G M2eGPI LoxP | 6078 | ToxoKO 2G M2eGPI LoxN | 5496 |
| targeted at mic3 locus | | targeted at mic3 locus | |
| ToxoKO 2G2G M2eGPI | 4850 | ToxoKO 2G2G M2eGPI | 5340 |
| LoxP random 1 | | LoxN random 1 | |
| ToxoKO 2G2G M2eGPI | 1998 | ToxoKO 2G2G M2eGPI | 3978 |
| LoxP random 2 | | LoxN random 2 | |
| ToxoKO 2G2G M2eGPI | 4561 | ToxoKO 2G2G M2eGPI | 4936 |
| LoxP random 3 | | LoxN random 3 | |
| ToxoKO 2G2G M2eGPI | 5237 | ToxoKO 2G2G M2eGPI | 5268 |
| LoxP random 4 | | LoxN random 4 | |
| ToxoKO 2G2G M2eGPI | 4778 | ToxoKO 2G2G M2eGPI | 4519 |
| LoxP random 5 | | LoxN random 5 | |
| | | ToxoKO 2G2G M2eGPI | 4899 |
| | | LoxN random 6 | |

This result shows that the Toxo tgmic1-3 KO strain is an expression vector optimized for transgene expression.

### EXAMPLE 6: EFFICACY STUDY OF THE NEO NCMIC1-3 KO-2G STRAIN IN THE PREVENTION OF LETHAL NEOSPOROSIS IN A MOUSE MODEL

In this study, the attenuated live strain Neo ncmic1-3 KO-2G was tested as a preventive vaccine for neosporosis. The objective is to determine whether or not the vaccine strain Neo ncmic1-3 KO-2G prevents dissemination and acute infection in a mouse model of lethal neosporosis.

Materials and Methods

Production of Neo Ncmic1-3 KO-2G Parasites and the Nc-1 Strain.

The parasites Neo ncmic1-3 KO-2G and Nc-1 (used for the challenge) are produced on a confluent mat of VERO cells (ATCC CCL81) in a complete medium containing DMEM 1.5 g/L NaHCO₃, 12% (v/v) SVF decompleted, 100 UI/mL Penicillin-100 UI/mL Streptomycin.

Before infection of the cell wall, the cells are maintained independently of parasites. Each time VERO cells are passed, the mat is washed three times with 5 mL HBSS, detached with 1 mL pure trypsin, re-suspended in full medium, distributed at a rate of 7.5,¹⁰⁵ cells per 25 cm2 of culture surface. The cells are incubated at 37° C. with 5% CO2 and are available for parasitic amplification three days later.

At D-4, the cell mat is infected with 1·10⁶ parasites per 25 cm² of confluence cell culture.

The vaccine parasites are harvested 4 days later. In short, the parasite-infected cell mat is washed with a complete medium to remove extracellular parasites. The cellular mat, containing the parasites, is scrapped and passed through a 27G syringe. The preparation is centrifuged for 10 min at 1500 g. The supernatant is removed and the pellet re-suspended in a DMEM solution with 0.1M Sucrose, 0.1M Trehalose, 2.5% inulin, 0.1M GSH, 1% proline and 1% ectoin added. The solution is prepared extemporaneously on the day of vaccination. The exact concentration of parasites, as well as viability, is estimated by counting in flow cytometry parasites with or without propidium iodide (P4864-10ML, Sigma). The final prepared solution was diluted to contain 2·10⁷ parasites per millilitre.

Design of the Study

Mice were immunized intraperitoneally (IP) to DO with a single dose of 1·10⁷ tachyzoites of the Neo ncmic1-3 KO-2G strain. Control mice were inoculated by the vehicle. Two mice from each genetic background were inoculated per group, or four mice per group.

At D60, immunized mice were infected intraperitoneally at the lethal dose of 2·10⁷ tachyzoites of virulent strain Nc-1. Two criteria are used to evaluate vaccine efficacy, mortality rate evaluation and humoral response against Neospora caninum at 30 days after vaccination.

Animals

Eight C57BL/6 and Balb/C mice with EOPS (Specific Pathogen Free) status were included in the study (Janvier Labs, . . . ). They have been raised in ventilated racks in an A2 animal house, in full compliance with current European ethical and regulatory standards.

IgG Determination by ELISA

The production of total serum specific IgG of N. caninum was determined by ELISA. The total parasitic extract of the Nc-1 strain was diluted in 0.05M pH9.6 carbonate/bicarbonate buffer to obtain a final concentration of 10 μg/mL. 100 μL per well of this mixture were deposited on a plate 96 flat-bottomed wells (Nunc MaxiSorp). After one night at +4° C., the wells were washed three times with 300 μL PBS buffer, Tween 20 0.05% (v/v), then saturated with 200 μL PBS, Tween 20 0.05%, BSA 4% (w/v) for 1 h30 at 37° C. After 3 washes with 300 μL per PBS well, Tween 20 0.05%, 100 μL per serum well of interest, previously diluted 1/50th in PBS, Tween 20 0.05%, were deposited on the plate and diluted in cascade by series of 2 in 2 (deposit on 11 wells/serum of interest). In control, (i) a serum, diluted in the same way as before and for which the specific total IgG titration is known and important, will serve as the reference control and (ii) a pool of serum diluted to 1/50th from mice inoculated by vehicles will serve as the negative control. Finally, 100 μL of PBS, Tween 20 0.05% were also deposited in 8 wells to serve as a “white” control. After 1 h incubation at 37° C. and three washes with 300 μL per PBS well, Tween 20 0.05%, 100 μL per well of the secondary anti-Mouse IgG antibody coupled to alkaline phosphatase (Sigma A3562) and diluted 1/5000e in PBS-Tween 20 0.05% were deposited. After 1 hour of incubation at 37° C. and three washes with 300 μL per well of PBS, Tween 20 0.05% followed by three washes with 300 μL per well of H2O mQ, the revelation was performed by adding 100 μL per well of a disodium para-nitrophenyl phosphate (PnPP) (Sigma) solution diluted at 1 mg/mL in a 1M Diethanolamine-HCl pH9.8 buffer. After 60 minutes of incubation at +24° C. and protected from light, the absorbance was measured at λ=405 nm with a counter reading at λ=620 nm using a plate reader (Infinite M200 Pro NanoQuant, Tecam). The D.O. values were subtracted from the average D.O. of the “white” control. Neospora caninum specific total serum IgG levels were expressed as antibody titres. Two methods were used for titration. The IgG titre is the inverse of the highest dilution of the serum of interest with an O.D. at least 2.5 times greater than that obtained with the negative control, or with an O.D. of 0.2.

Results

The efficacy of Neo ncmic1-3 KO-2G was estimated on a lethal wall model.

Clinical Sign

After vaccination, the vaccinated animals showed no specific clinical signs, no weight alterations and no significant increase in body temperature, suggesting a good tolerance of mice to the Neo ncmic1-3 KO-2G strain.

Evaluation of the Humoral Response

At 30 days after vaccination, blood samples were taken and the specific IgG antibody titre was measured by ELISA to estimate the quality of the immune response post-vaccination. The results are presented in FIG. 12.

None of the unvaccinated mice showed a positive antibody titre. Conversely, all vaccinated mice, regardless of their genetic background, seroconverted and showed an antibody titre significantly different from that of unvaccinated animals.

Effectiveness of Vaccination on Mouse Survival

The real effectiveness of vaccination is assessed by an infectious challenge with a virulent strain (Nc-1). At D60 days after vaccination, mice were inoculated with the wild strain Nc-1 at a lethal dose of 2·10⁷ tachyzoites per animal. All mice that received the control solution died during the experiment, i.e. 100% of lethality. In contrast, 100% of vaccinated mice survived until the end of the experiment, sixty days later. In addition, none of these vaccinated mice showed significant clinical signs, suggesting a complete protective effect on both morbidity and mortality.

The results of this experiment demonstrate a clear immune response and protective effect of the Neo ncmic1-3 KO-2G-based vaccine against a lethal challenge with a wild Neospora caninum Nc-1 strain.

### EXAMPLE 7: EFFICACY STUDY OF THE TOXO TGMIC1-3 KO-2G STRAIN IN THE PREVENTION OF CONGENITAL TOXOPLASMOSIS IN A MOUSE MODEL

The main purpose of this study is to study the efficacy of the vaccine strain Toxo tgmic1-3 KO-2G against congenital toxoplasmosis in a mouse model.

Materials and Methods

Parasite Production Toxo Tgmic1-3 KO-2G

Toxo tgmic1-3 KO-2G strains are produced in human fibroblasts (HFF Hs27 ATCC CRL-1634) grown in minimal Dulbecco medium (DMEM) supplemented with 12% Australian fetal calf serum (SVF aust). The tachyzoites were collected from the supernatant. The parasites were listed on Malassez cell and diluted in DMEM medium to a concentration of 500 parasites per mL.

Design of the Study

Vaccination (100 tachyzoites in a volume of 200 μL of DMEM) is performed subcutaneously on female mice at DO with a 27G needle.

Before injection (D-2) and 28 days after injection, peripheral venous blood was collected and left at room temperature for a minimum of one hour. The serum was obtained by centrifugation at 5000 g for 10 minutes. The prepared seras were stored at −20° C.

Eight weeks after vaccination, 23 seroconverted mice (for T. gondii) per batch were placed in males and then challenged mid-pregnancy per os with 15 cysts of T. gondii strain 76K. The mothers are then isolated and the calving followed. The mice are followed for a period of about a month and then euthanized. A small number of mice will be analyzed after sacrifice. Then the mothers will be sacrificed, their spleens re-cultured to study the cellular response.

Animals

The number of mice per batch required for the study is 16 pregnant mice to allow a statistical study. The analysis of the immune response in 16 pregnant mice requires vaccinating a total of 50 mice per batch, taking into account the following parameters: gestation yield and potential mortality associated with the injection of the parasite (the mouse is an animal relatively sensitive to an injection of Toxoplasma gondii and vaccination to obtain effective seroconversion can generate mortality). Thus the vaccinated batches contain 50 animals at the beginning. Mice from batches 1 and 2 were not vaccinated, so 23 mice were sufficient for these batches. The analysis is performed on 16 pregnant mice per batch. The supernumerary mice are sacrificed.

TABLE 20
Distribution of animals in the different batches.

| | | | | Number of | |
|---|---|---|---|---|---|
| | | | Initial | people of | Number of |
| | | | number of | female | pregnant |
| | | | female | mice to | females |
| | Vaccination | challenge | mice | mate | required |
| Batch 1: non-vaccinated, non | — | — | 23 | 23 | 16 |
| challenged batch (control) | | | | | |
| Batch 2: non-vaccinated | — | Feeding | 23 | 23 | 16 |
| batch - challenged | | 15 cysts | | | |
| Batch 3: vaccinated | 100 tachyzoites Toxo | — | 50 | 23 | 16 |
| batch - not challenged | tgmic1-3 KO - 2G | | | | |
| Batch 4: vaccinated | 100 tachyzoites Toxo | Feeding | 50 | 23 | 16 |
| batch - challenged | tgmic1-3 KO - 2G | 15 cysts | | | |

A total of 146 8-week-old female SWISS mice were included in the study and distributed in different batches (Table 20). The animals were housed and handled in strict compliance with the ethical standards in force in France and the breeding standards imposed by European regulations. Food and drinking water have been distributed ad-libitum throughout the experiment.

After a period of 7 days of acclimatization, the mice were individually identified and randomly assigned to 4 groups.

IgG and IgM Assay by ELISA

A 96-well plate was adsorbed with protein extract from the RH strain of Toxoplasma gondii (10 μg/ml) diluted in carbonate buffer pH 9.6 overnight at 4° C. After 2 washes in PBS 1×/Tween 0.05% and one wash in PBS 1×, the plate was saturated with PBS 3% BSA for 1 h30 at room temperature. Then, for the titration, the series 2 dilutions of 2 of 2 in, in PBS+3% BSA of the murine compounds to be tested were removed.

After 1 h incubation at 37° C., the plate was washed twice (PBS 1×/Tween 0.05%), then the secondary antibody (anti-mouse IgG coupled to alkaline phosphatase (Sigma A3562) or anti-mouse IgM coupled to alkaline phosphatase (Sigma A9688) is added and diluted to 1/30 000 to PBS 1×/Tween 0.05%, before a new incubation at 37° C. for 1 h30. After 2 washes in PBS 1×/Tween 0.05% and one wash in PBS 1×, pNPP (4-Nitrophenyl phosphate disodium salt hexahydrate) was used at a concentration of 1 mg/ml diluted in DEA-HCL buffer for revelation, and the plate was incubated for 15 to 20 minutes at room temperature. The reaction is stopped with the addition of 3N NaOH stop solution. The absorbance at 405 nm was read via the Infinite Nanoquant Plate Reader M200 PRO (TECAN).

ELISA Determination of IFN-γ Secreted by Splenocytes Stimulated In Vitro

After each sampling, the spleen was crushed on a 100 μm sieve placed above a 50 ml Falcon tube with the addition of 5 ml RPMI medium. The shred was then centrifuged for 10 min at 400 g. The pellet was taken up in 300 μl RPMI and then 1 ml lysis buffer was added. The tube was incubated for 3 min at 4° C., and the reaction was stopped with the addition of 2% excess PBS 1×/SVF, then the cells were centrifuged for 10 min at 400 g. The cell pellets obtained were then taken up in 5 ml of stimulation medium. The cells were enumerated on Malassez cells in the presence of trypan blue which colours the dead cells.

The cells were cultured in 96 wells in plates at a rate of 5′¹⁰⁵ cells/well. Different stimulants could be added: antigenic extracts of the RH strain of Toxoplasma gondii (10 μg/ml) or concanavalin A (5 μg/ml) as positive controls. The plates were cultured in an oven at 37° C., 5%_{CO2} for 72 hours.

The IFN-γ assay was performed on splenocyte culture supernatants in the presence of these different stimulants. The assay was performed by an ELISA test according to the protocol of the kit “Mouse IFN-γ ELISA Ready-set-go® (Ebiosciences).

Evaluation of the Number of Cysts Present in the Brains of Mice

The brains of mothers and mice are sampled to determine the presence or absence of brain cysts. Following the sacrifice of the mice, the brains are removed and crushed. The number of cysts is then counted by microscopic observation of the entire brain crushed material.

Results

Effectiveness of Vaccination on Mother Mice

Survival and Clinical Signs Following Vaccination with the Toxo Tgmic1-3 KO-2G Strain

Mice are observed daily from vaccination (DO) until 33rd day after vaccination and clinical signs are noted. An overall clinical score is calculated according to Table 21 of the sign score table. The results are presented in FIG. 13-A.

TABLE 21
Scoreboard of clinical signs observed for mice

| | Score/2 |
|---|---|
| Physical appearance | |
| Normal | 0 |
| Brushed hair | 1 |
| Severe piloting | 2 |
| Behaviour/mobility | |
| Normal | 0 |
| Slight prostration/reduced mobility | 1 |
| Immobile | 2 |
| Weight | |
| <10% loss | 0 |
| 10% to 20% loss | 1 |
| >20% loss | 2 |
| Injury at the injection site | |
| No injury | 0 |
| Papule/redness | 1 |
| Ulceration/necrosis | 2 |

Mice from unvaccinated batches did not show clinical signs, while mice from vaccinated batches showed some moderate clinical signs with a clinical score of less than 2. The peak of clinical signs was observed on D18. These clinical signs were observed up to D33. Mortality in SWISS mice can be observed depending on the injection site and parasitic strains of mortality. A survival rate of 90% (10 dead mice/100 mice for batches 3 and 4) is obtained at D30 for vaccination of mice with the Toxo tgmic1-3 KO-2G strain.

Evaluation of the Adaptive Humoral Response

The determination of serum IgG against Toxoplasma gondii proteins shows very clearly that the serum IgG from mice before injection does not produce antibodies specific to this strain.

Vaccination with the Toxo tgmic1-3 KO-2G strain allows a high IgG production at D28 (batch 3 and 4) and J129 (batch 3). As expected, the challenge (strain 76K) of unvaccinated mice (batch 2) also allows IgG production. The production of IgG for the vaccinated batch is reinforced by the challenge (batch 4 to D129). The results are presented in FIG. 13-B.

Evaluation of the Specific Cellular Response

In order to evaluate the development of a cellular immune response, IFN-γ (cytokine indicating the development of a Th1-type response) secreted in splenocyte culture supernatants following various stimuli, was measured 72 hours after culture.

As expected, the concentration of IFN-γ is increased after a challenge compared to the control group. For all mice vaccinated with Toxo tgmic1-3 KO-2G (having been challenged (batch 4) or not (batch 3)), significant concentrations of IFN-γ are measured when the cells are stimulated with antigenic extracts of the RH strain of Toxoplasma gondii, compared to unvaccinated mice. This suggests a vaccination-induced cellular response. The results are presented in FIG. 13-C.

Counting the Number of Cysts Present in the Brains of Mother Mice

The mothers were sacrificed on D129. The number of cysts is obtained by microscopic observation of the entire brain crushed material (4 brains per batch). No cysts are detected for batch 1 (unvaccinated, not challenged) while after challenge, an average of 2250.75 cysts per brain is obtained for batch 2 (unvaccinated, challenged). In batch 3 (only vaccinated), a very small number of cysts are observed (0.75 cysts per brain). In batch 4 (vaccinated and challenged), there was a significant decrease in the number of cysts compared to the unvaccinated batch (batch 2), with an average of 52 cysts per brain.

Mice vaccinated with the Toxo tgmic1-3 KO-2G strain form very few brain cysts following a challenge with the T. gondii 76K strain. The results are presented in FIG. 13-D.

Effectiveness of Vaccination on Mice

Effectiveness on Proliferation and Sex Ratio

Similar prolificity was obtained for all groups showing that vaccination and/or challenge did not affect the number of mice per litter. On the other hand, a change in sex ratio after T. gondii infection has already been described (Kankova et al, parasitology 2007). We observe a reduced number of males in the control group after challenge while this is not observed for the vaccinated group, suggesting that vaccination prevents sex ratio change. The results are presented in FIGS. 13-E and 13-F.

Effectiveness on Stillbirths and Mortality

Stillbirths and deaths were increased following the challenge in the absence of vaccination (batch 2 compared to batch 1). For the vaccinated batch only (batch 3), stillbirth and mortality are at a very low level, close to the control group (batch 1). The vaccinated and challenged group (batch 4) shows a significant reduction in the number of stillbirths and mortality compared to the unvaccinated and challenged group (batch 2). Vaccinating mothers therefore protects the offspring from an increase in stillbirths and mortality following a challenge. The results are presented in FIGS. 13-G and 13-H.

Effectiveness on Mouse Survival

The mice were followed over a 32-day period to study the effectiveness of the vaccination. Survival of mice decreases significantly for the batch whose mothers were challenged but not vaccinated. The other groups have a survival rate close to that obtained for the control batch (batch 1). Vaccinating mothers therefore increases the survival rate of mice following a challenge during gestation. The results are presented in FIG. 13-I.

Effectiveness on the Clinical Signs of Mice.

Mice are observed daily for 30 days and clinical signs are noted. An overall clinical score is calculated according to the sign score table (see Table 22 below).

TABLE 22
Scoreboard of clinical signs observed for mice

| | Score/2 |
|---|---|
| Mobility | |
| Normal | 0 |
| Limited | 1 |
| Immobile | 2 |
| | Score/1 |
| Isolation | |
| No isolation | 0 |
| Isolation of | 1 |
| mother/nest/mice | |
| Growth | |
| Normal | 0 |
| Slower growth | 1 |

The mice from the challenged group (batch 2) have a higher clinical score than the mice from the control group (batch 1). This clinical score is due in particular to a slower growth of mice in this group. For the other batches (vaccinated or vaccinated and challenged), the clinical score is similar to the control group. The vaccination of mothers prevented growth retardation induced by the challenge in mice. The results are presented in FIG. 13-J.

Evaluation of the Immature Humoral Response: IgM

IgM cannot pass through the placental barrier but could pass to offspring via breast milk. Since IgM has a half-life of 5 days, the 32-day IgM assay reflects the immunity of mice developed in response to T. gondii (vaccine or challenge strain) transmitted by the mother. The humoral response developed by mice (in response to vaccination or the mother's challenge) is therefore evaluated by ELISA assay of IgM immunoglubulin at 32 days of age. The results are presented in FIG. 13-K.

The IgM titre is increased for mice in the group from challenged mice (batch 2) compared to those from non-challenged mice (batch 1). For the vaccinated group only (batch 3), the IgM titre is close to the detection limit, indicating that there is no vertical transmission from the strain to the mice. For the vaccinated and challenged group (batch 4), the IgM titre measured for mice is significantly reduced compared to the titre obtained for mice from the challenged group (batch 2). Vaccination of mice has thus made it possible to reduce vertical transmission from the challenge strain to mice.

The Toxo tgmic1-3 KO-2G strain thus allowed the implementation in mice of a humoral and cellular immune response directed against the T. gondii parasite. Vaccination of mice with the Toxo tgmic1-3 KO-2G strain resulted in a significant decrease in the cerebral parasitic load observed after a challenge with the virulent 76K strain. In addition, the Toxo tgmic1-3 KO-2G strain has good safety.

Finally, the Toxo tgmic1-3 KO-2G strain allowed to protect the offspring from congenital toxoplasmosis (after a challenge with the virulent 76K strain at mid-gestation) with a decrease in stillbirth and mortality, a better clinical score for mice.

### EXAMPLE 8: SAFETY AND IMMUNOGENICITY STUDY OF TOXO TGMIC1-3 KO-2G STRAINS EXPRESSING THE M2EGPI ANTIGEN OF INFLUENZA VIRUS IN A MOUSE MODEL

The objective of this experiment is to determine whether the Toxo tgmic1-3 KO-2G strain expressing the M2eGPI antigen of Influenza virus provides a humoral and cellular immune response to the Toxoplasma gondii vector and against the targeted viral antigen.

Material and Method

Production of Toxo Tgmic1-3 KO-2G Parasites Expressing the Influenza Virus M2eGPI Antigen

Toxo tgmic1-3 KO-2G strains expressing influenza virus antigen (Toxo tgmic1-3 KO-2G M2eGPI LoxP, Toxo tgmic1-3 KO-2G M2eGPI LoxN and Toxo tgmic1-3 KO-2G M2eGPI total population-random insertion—example 4) are produced in human fibroblasts (HFF Hs27 ATCC CRL-1634) grown in minimal medium of Dulbecco (DMEM) supplemented by 10% fetal calf serum (SVF), 2 mM glutamine, 100 U/mL penicillin and 100 U/mL streptomycin. The tachyzoites were collected after mechanical lysis of the host cells by 3 passages in 25G syringes. The parasites were listed on Malassez cell and diluted in DMEM medium to a concentration of 500 parasites per mL.

Animals

A total of 45 6-week-old female SWISS mice were included in the study. The animals were housed and handled in strict compliance with the ethical standards in force in France and the breeding standards imposed by European regulations. Food and drinking water were distributed ad-libitum throughout the experiment.

After a 15-day acclimatization period, the mice were individually identified and randomly assigned to 5 groups.

Batch 1: Toxo tgmic1-3 KO-2G (n=10)

Batch 2: Toxo tgmic1-3 KO-2G M2eGPI LoxP (n=10)

Batch 3: Toxo tgmic1-3 KO-2G M2eGPI LoxN (n=10)

Batch 4: Toxo tgmic1-3 KO-2G M2eGPI total population-random insertion (n=10)

Batch 5: naive (n=5)

At D0, 100 tachyzoites were injected intraperitoneally into a volume of 200 μL of DMEM. The animals were then followed and sacrificed 5 weeks after injection (J35) and the rats were collected. Before (D-1) and 34 days after injection, peripheral venous blood was collected and left at room temperature for a minimum of 30 minutes. The serum was obtained by centrifugation at 3000 g for 10 minutes. The prepared seras were stored at −20° C.

IgG Determination by ELISA

A 96-well plate was adsorbed with a mixture of 4 synthetic M2e peptides at 10 μg/mL (Anaspec) corresponding to the 4 M2e present in the construction of the fusion protein sag1M2eGPÏ or the protein extract of Toxo tgmic1-3 KO-2G (10 μg/ml) diluted in carbonate buffer pH 9.6 overnight at 4° C. After washing with PBS 1×/Tween 0.05%, the plate was saturated with PBS 1×/Tween 0.05%-BSA 4% for 1 h30 at 37° C. Then, for the titration, the series 2 dilutions of 2 of 2 in, in PBS 1×/Tween 0.05% of the murine samples to be tested were removed. After 1 h incubation at 37° C., the plate was washed, then the secondary antibody (IgG anti-mouse IgG coupled to alkaline phosphatase) is added and diluted to 1/30 000 PBS 1×/Tween 0.05%, before further incubation at 37° C. for 1 h30. After washing, pNPP was used at a concentration of 1 mg/ml diluted in DEA-HCL buffer for revelation, and the plate was incubated for 1 h at 37° C. The absorbance at 405 nm of each well with a counter reading at 620 nm was read via the Infinite Nanoquant Plate Reader M200 PRO (TECAN).

ELISA Determination of IFN-□ Secreted by Splenocytes Stimulated In Vitro

After each sampling, the spleen was crushed on a 100 μm sieve placed above a 50 ml Falcon tube with the addition of 5 ml RPMI medium. The shred was then centrifuged for 10 min at 400 g. The pellet was taken up in 300 μl RPMI and then 1 ml lysis buffer was added. The tube was incubated for 3 min at 4° C., and the reaction was stopped with the addition of 2% excess PBS 1×/SVF, then the cells were centrifuged for 10 min at 400 g. The cell pellets obtained were then taken up in 5 ml of stimulation medium. The cells were enumerated on Malassez cells in the presence of trypan blue which colours the dead cells.

The cells were cultured in 96 wells in plates at a rate of 5′¹⁰⁵ cells/well. Different stimulants could be added: antigenic extracts of Toxo tgmic1-3 KO-2G (10 μg/ml), a mix of 4 synthetic M2e peptides at 10 μg/mL (Anaspec) corresponding to the 4 M2e present in the construction of the fusion protein sag1M2eGPI or concanavaline A (5 μg/ml) which serves as positive control. The plates were cultured in an oven at 37° C., 5%_{CO2} for 72 hours. The IFN-γ assay was performed on splenocyte culture supernatants in the presence of different stimulants. The assay was performed by an ELISA test according to the protocol of the kit “Mouse IFN-γ ELISA Ready-set-go® (Ebiosciences).

Results

Survival and Clinical Signs

Most mice show clinical signs such as tousled hair, swelling of the abdomen and prostration, with the exception of mice that have not received parasitic strains.

Evaluation of the Adaptive Humoral Response to the Vector Toxo Tgmic1-3 KO-2G

The determination of serum IgG against Toxo tgmic1-3 KO-2G proteins shows very clearly that they will come from mice before injection and do not produce antibodies specific to this strain. Concerning the samples will be collected 34 days after injection, a strong increase in absorbance synonymous with mouse seroconversion following vaccination with Toxo tgmic1-3 KO-2G or one of the derived strains is observed. A large majority of mice are seroconverted for toxoplasmosis.

Evaluation of the Adaptive Humoral Response to the M2e Influenza Antigen

Following the injection of Toxo tgmic1-3 KO-2G strains expressing M2eGPI, antibodies specific to the M2e antigen expressed by Toxo tgmic1-3 KO-2G M2eGPI LoxP, Toxo tgmic1-3 KO-2G M2eGPI LoxN and Toxo tgmic1-3 KO-2G M2eGPI total population-random insertion, but no production of antibodies specific for the M2e antigen for the Toxo tgmic1-3 KO-2G strain.

The expression of the M2eGPI transgene being similar or slightly higher for clones with a targeted insertion of the transgene (located at the LoxP and LoxN scars) than for populations with a random insertion of the transgene (example 4), a similar or slightly better adaptive humoral response to the antigen is obtained for Toxo tgmic1-3 KO-2G M2eGPI LoxP, Toxo tgmic1-3 KO-2G M2eGPI LoxN strains than for populations where the transgene is randomly inserted.

Evaluation of the Specific Cellular Response of Toxo Tgmic1-3 KO-2G or Influenza M2e Antigen

In order to evaluate the development of a cellular immune response, IFN-γ (cytokine indicating the development of a Th1-type response) secreted in splenocyte culture supernatants following various stimuli, was measured 72 hours after culture.

For all mice vaccinated with Toxo tgmic1-3 KO-2G or Toxo tgmic1-3 KO-2G M2eGPI (targeted insertion in LoxP, LoxN or randomly integrated), significant concentrations of IFN-γ are measured when the cells are stimulated with antigenic extracts of Toxo tgmic1-3 KO-2G, compared to unvaccinated mice.

Concerning cells restimulated with M2e, no production of IFN-γ is detectable for all mice, whether naïve, vaccinated with Toxo tgmic1-3 KO-2G or with Toxo tgmic1-3 KO-2G M2eGPI (targeted insertion in LoxP, LoxN or random integration).

It was therefore observed the implementation of a humoral immune response to the M2eGPI construction and a humoral and cellular immune response directed against the vector Toxo tgmic1-3 KO-2G.

1-15. (canceled)

16. A mutant Sarcocystidae strain in which the two genes mic1 and mic3 are deleted containing two sites of specific recombination of an enzyme allowing specific recombination, said specific recombination sites being at the respective locus of each of the said deleted genes, the site of specific recombination of the enzyme allowing specific recombination at the locus of the deleted mic1 gene being different to that at the locus of the deleted mic3 gene.

17. The mutant strain according to claim 16, wherein said mutant strain is a strain of the genus Neospora spp.

18. The mutant strain according to claim 16, wherein said mutant strain is a strain of the species Neospora caninum.

19. The mutant strain according to claim 16, wherein said mutant strain is a strain of the genus Toxoplasma spp.

20. The mutant strain according to claim 16, wherein said mutant strain is a strain of the species Toxoplasma gondii.

21. The mutant strain according to claim 16, wherein both mic1 and mic3 genes are deleted, containing two specific recombination sites of an enzyme allowing specific recombination, at the respective locus of each of said deleted genes, the specific recombination site of the enzyme allowing specific recombination, at the locus of the deleted mic1 gene being different from that at the locus of the deleted mic3 gene, and not containing heterologous DNA, other than heterologous DNA corresponding to the specific recombination sites of the enzyme allowing specific recombination, at the respective locus of each of the said deleted genes.

22. The mutant strain according to claim 16, wherein both mic1 and mic3 genes are deleted, containing two specific recombination sites of an enzyme allowing specific recombination, at the respective locus of each of said deleted genes, the specific recombination site of the enzyme allowing specific recombination, at the locus of the deleted mic1 gene being different from that at the locus of the deleted mic3 gene, and not containing heterologous DNA, other than heterologous DNA corresponding to the specific recombination sites of the enzyme allowing specific recombination, at the respective locus of each of the said deleted genes,
said enzyme allowing a specific recombination being the Cre-recombinase, and the specific recombination sites of Cre-recombinase at the respective locus of each of said deleted genes being selected from: LoxN of SEQ ID NO: 5, LoxP of SEQ ID NO: 12 and Lox2272 of SEQ ID NO: 68.

23. The mutant strain according to claim 16, said strain containing heterologous DNA at the locus of the deleted mic1 gene or at the locus of the deleted mic3 gene, different from heterologous DNA corresponding to the enzyme-specific recombination sites allowing recombination specific to the respective locus of each of said deleted genes,
said heterologous DNA being flanked:
a first enzyme-specific recombination site allowing specific recombination, corresponding to the enzyme-specific recombination site allowing specific recombination at the locus of the deleted mic1 gene or the locus of the deleted mic3 gene and,
a second specific recombination site identical to said first specific recombination site, corresponding to the enzyme-specific recombination site allowing specific recombination, at the locus of the deleted mic1 gene or the locus of the deleted mic3 gene and,
and the means necessary for its transcription.

24. The mutant strain according to claim 16, said strain containing heterologous DNA at the locus of the deleted mic1 gene or at the locus of the deleted mic3 gene, different from heterologous DNA corresponding to the specific recombination sites of the enzyme allowing recombination specific to the respective locus of each of said deleted mic1 and mic3 genes, said heterologous DNA coding for a protein and being flanked:
a first enzyme-specific recombination site allowing specific recombination, corresponding to the enzyme-specific recombination site allowing specific recombination at the locus of the deleted mic1 gene or the locus of the deleted mic3 gene and,
a second specific recombination site identical to said first specific recombination site, corresponding to the enzyme-specific recombination site allowing specific recombination, at the locus of the deleted mic1 gene or the locus of the deleted mic3 gene and,
and the means necessary for the protein expression of the said heterologous DNA,
said enzyme allowing specific recombination being Cre-recombinase and the specific recombination sites of Cre-recombinase being chosen from: LoxN of SEQ ID NO: 5, LoxP of SEQ ID NO: 12 and Lox2272 of SEQ ID NO: 68.

25. The mutant strain according to claim 16, containing heterologous DNA at the locus of the deleted mic1 gene or at the locus of the deleted mic3 gene, different from heterologous DNA corresponding to the specific recombination sites of an enzyme allowing specific recombination, said enzyme being the Cre-recombinase, at the respective locus of each of the said mic1 and mic3 genes deleted
and containing three specific recombination sites which are respectively:
site A corresponding to the specific recombination site of the
Cre-recombinase at the locus of the deleted mic1 gene,
site B corresponding to the specific recombination site of Cre-recombinase at the locus of the deleted mic3 gene, and
site C corresponding to said second specific recombination site which flanks said second heterologous DNA identical to said first specific recombination site, said first Cre-recombinase specific recombination site corresponding to said Cre-recombinase specific recombination site at the locus of the deleted mic1 gene (site A) or said Cre-recombinase specific recombination site at the locus of the deleted mic3 gene (site B)
said three specific recombination sites A, B and C being selected from the following sites: LoxN of SEQ ID NO: 5, LoxP of SEQ ID NO: 12 and Lox2272 of SEQ ID NO: 68, so that
said site A and said site B are different, and
said site C is identical to said site A or said site B.

26. The mutant strain according to claim 16, containing heterologous DNA at the locus of the deleted mic1 gene or at the locus of the deleted mic3 gene, different from heterologous DNA corresponding to the specific recombination sites of an enzyme allowing specific recombination, said enzyme being the Cre-recombinase, at the respective locus of each of the said mic1 and mic3 genes deleted
and containing three specific recombination sites which are respectively:
site A corresponding to the specific recombination site of the
Cre-recombinase at the locus of the deleted mic1 gene,
site B corresponding to the specific recombination site of Cre-recombinase at the locus of the deleted mic3 gene, and
site C corresponding to said second specific recombination site which flanks said second heterologous DNA identical to said first specific recombination site, said first Cre-recombinase specific recombination site corresponding to said Cre-recombinase specific recombination site at the locus of the deleted mic1 gene (site A) or said Cre-recombinase specific recombination site at the locus of the deleted mic3 gene (site B)
said three specific recombination sites A, B and C being selected from the following sites: LoxN of SEQ ID NO: 5, LoxP of SEQ ID NO: 12 and Lox2272 of SEQ ID NO: 68, so that
said site A and said site B are different, and
said site C is identical to said site A or said site B;
such as
said site A is the LoxN site of SEQ ID NO: 5
said site B is the LoxP site of SEQ ID NO: 12, and
said site C is the LoxN site of SEQ ID NO: 5, said first specific recombination site of Cre-recombinase being site A;
or such as
said site A is the LoxN site of SEQ ID NO: 5
said site B is the LoxP site of SEQ ID NO: 12, and
said site C is the LoxP site of SEQ ID NO: 12, said first specific recombination site of Cre-recombinase being site B,
or such as
said site A is the LoxP site of SEQ ID NO: 12
said site B is site LoxN of SEQ ID NO: 5, and
said site C is the LoxP site of SEQ ID NO: 12, said first specific recombination site of Cre-recombinase being site A;
or such as
said site A is the LoxP site of SEQ ID NO: 12
said site B is site LoxN of SEQ ID NO: 5, and
said site C is the LoxN site of SEQ ID NO: 5, said first specific recombination site of Cre-recombinase being site B.

27. The mutant strain according to claim 16, wherein said mutant strain is a mutant strain of Toxoplasma spp.,
and wherein the rop16I gene is deleted and contains an enzyme-specific recombination site allowing locus-specific recombination of said deleted rop16I gene, said site being different from said specific recombination site located at the locus of the deleted mic1 gene and said specific recombination site located at the locus of the deleted mica gene
and said mutant strain comprising a gene encoding the protein GRA15II, as well as the means necessary for the expression of said protein at the locus of said deleted rop16I gene,
said gene encoding the protein GRA15II at the locus of said deleted rop16I gene, being flanked upstream by said enzyme-specific recombination site allowing locus-specific recombination of said deleted rop16I gene.

28. The mutant strain according to claim 16, wherein said mutant strain is a mutant strain of Toxoplasma spp.,
and wherein the rop16I gene is deleted and contains an enzyme-specific recombination site allowing locus-specific recombination of said deleted rop16I gene, said site being different from said specific recombination site located at the locus of the deleted mic1 gene and said specific recombination site located at the locus of the deleted mica gene
and said mutant strain comprising a gene encoding the protein GRA15II, as well as the means necessary for the expression of said protein at the locus of said deleted rop16I gene,
said gene encoding the protein GRA15II at the locus of said deleted rop16I gene, being flanked upstream by said enzyme-specific recombination site allowing locus-specific recombination of said deleted rop16I gene, in which said enzyme allowing specific recombination is Cre-recombinase, and said specific recombination site of an enzyme allowing locus-specific recombination of said deleted rop16I gene is a specific recombination site of Cre-recombinase chosen from the following sites: LoxN of SEQ ID NO: 5, LoxP of SEQ ID NO: 12 and Lox2272 of SEQ ID NO: 68,
this specific recombination site being different from the specific recombination sites of Cre-recombinase at the locus of the deleted mic1 gene and at the locus of the deleted mic3 gene, said strain containing three specific recombination sites which are respectively:
site A corresponding to the specific recombination site of the Cre-recombinase at the locus of the deleted mic1 gene
site B corresponding to the specific recombination site of Cre-recombinase at the locus of the deleted mic3 gene, and
site D corresponding to the specific recombination site of the Cre-recombinase at the locus of said deleted rop16I gene
such as
said site A is the LoxN site of SEQ ID NO: 5,
said site B is the LoxP site of SEQ ID NO: 12,
said site D is the Lox2272 site of SEQ ID NO: 68.

29. The mutant strain according to claim 16, wherein said mutant strain is a mutant strain of Toxoplasma spp., said enzyme allowing specific recombination is Cre-recombinase, in which the rop16I gene is deleted and said strain contains a specific recombination site of the Cre-recombinase, at the locus of said deleted rop16I gene,
said strain containing four specific recombination sites which are respectively:
site A corresponding to the specific recombination site of the Cre-recombinase at the locus of the deleted mic1 gene
site B corresponding to the specific recombination site of Cre-recombinase at the locus of the deleted mic3 gene, and
the site C corresponding to said second specific recombination site which flanks said heterologous DNA identical to said first specific recombination site, said first Cre-recombinase specific recombination site corresponding to said Cre-recombinase specific recombination site at the locus of the deleted mic1 gene (site A) or said Cre-recombinase specific recombination site at the locus of the deleted mica gene (site B)
site D corresponding to the specific recombination site of the Cre-recombinase at the locus of said deleted rop16I gene
such as
said site A is the LoxN site of SEQ ID NO: 5
said site B is the LoxP site of SEQ ID NO: 12,
said site C is the LoxN site of SEQ ID NO: 5, said first specific recombination site of Cre-recombinase being site A, and
said site D is the Lox2272 site of SEQ ID NO: 68;
or such as
said site A is the LoxN site of SEQ ID NO: 5
said site B is the LoxP site of SEQ ID NO: 12,
said site C is the LoxP site of SEQ ID NO: 12, said first specific recombination site of Cre-recombinase being site B, and
said site D is the Lox2272 site of SEQ ID NO: 68.

30. The mutant strain according to claim 16, wherein said heterologous DNA encodes an immunogenic heterologous antigen of virus, parasite or bacterium.

31. The mutant strain according to claim 16, wherein said heterologous DNA consists of the nucleotide sequence SEQ ID NO: 173, encoding the influenza virus antigen SEQ ID NO: 167

32. The mutant strain according to claim 16, wherein said mutant strain is a strain of Toxoplasma gondii selected from
a strain in which
the mic1 gene and the mic3 gene are deleted and wherein the specific recombination site of the Cre-recombinase at the locus of the deleted mic1 gene is the LoxN site of SEQ ID NO: 5 and
the specific recombination site of Cre-recombinase at locus of the deleted mic3 gene is the LoxP site of SEQ ID NO: 12.
a strain in which the mic 1 gene, the mic 3 gene and the rop16I gene are deleted, and comprising a gene encoding the protein GRA15II, as well as the means necessary for the expression of said protein, at the locus of said deleted rop16I gene,
said gene encoding the protein GRA15II at the locus of said deleted rop16I gene, being flanked upstream by a specific recombination site of the Cre-recombinase,
in which
the specific recombination site of Cre-recombinase at the locus of the deleted mic1 gene is the LoxN site of SEQ ID NO: 5, and
the specific recombination site of Cre-recombinase at the locus of the deleted mic3 gene is the LoxP site of SEQ ID NO: 12, and
said Cre-recombinase specific recombination site which upstream flanks said gene encoding the GRA15II protein at the locus of the deleted rop16I gene, is the Lox2272 site of SEQ ID NO: 68.

33. The mutant strain according to claim 16, wherein said mutant strain is a Neospora caninum strain and wherein the mic1 gene and the mic3 gene are deleted and wherein
the specific recombination site of Cre-recombinase at the locus of the deleted mic1 gene is the LoxP site of SEQ ID NO: 12 and
the specific recombination site of Cre-recombinase at the locus of the deleted mic3 gene is the LoxN site of SEQ ID NO: 5.

34. A method for the targeted insertion of heterologous DNA using a mutant strain according to claim 16, not containing heterologous DNA different from heterologous DNA corresponding to the specific recombination sites of Cre-recombinase at the respective locus of each of said deleted genes.

35. A mutant strain of Toxoplasma spp. wherein the genes mic1, mic3 and rop16I are deleted, containing three Cre-recombinase specific recombination sites, at the respective locus of each of said deleted genes, the Cre-recombinase specific recombination site, at the locus of the deleted mic1 gene being different from that at the locus of the deleted mic3 gene and the recombination site specific to the locus of the deleted rop16I gene being different from the recombination sites specific to the loci of the deleted mic1 and mic3 genes and said strain containing DNA heterologous to the locus of the deleted mic1 gene or the locus of the deleted mic3 gene or the locus of the deleted rop16I gene, said heterologous DNA being different from heterologous DNA corresponding to the specific recombination sites of the Cre-recombinase, at the respective locus of each of the deleted mic1, mic3 and rop16I genes,
in such a way that:
when said heterologous DNA is inserted at the locus of the deleted mic1 gene, it is flanked:
a first Cre-recombinase-specific recombination site, corresponding to a Cre-recombinase-specific recombination site, at the locus of the deleted mic1 gene (site A), and
a second Cre-recombinase specific recombination site (site C), identical to the first Cre-recombinase specific recombination site located at the locus of the deleted mic1 gene (site A),
when said heterologous DNA is inserted at the locus of the deleted mic3 gene, it is flanked:
a first Cre-recombinase-specific recombination site, corresponding to a Cre-recombinase-specific recombination site, at the locus of the deleted mic3 gene (site B), and
a second Cre-recombinase specific recombination site (site C), identical to the first Cre-recombinase specific recombination site located at the locus of the deleted mic3 gene (site B),
when said heterologous DNA is inserted at the locus of the deleted rop16I gene, it is flanked:
a first Cre-recombinase-specific recombination site, corresponding to a Cre-recombinase-specific recombination site, at the locus of the deleted rop16I gene (site D), and
a second Cre-recombinase specific recombination site (site C), identical to the first Cre-recombinase specific recombination site located at the locus of the deleted rop16I gene (site D),
said strain comprising the elements necessary for the transcription of said heterologous DNA, or the means necessary for the expression of said heterologous DNA when said heterologous DNA encodes at least one protein,
said mutant strain then containing four specific recombination sites of the Cre-recombinase defined as follows:
site A corresponding to said specific recombination site of the Cre-recombinase at the locus of the deleted mic1 gene
site B corresponding to said specific recombination site of the Cre-recombinase at the locus of the deleted mic3 gene, and
site C corresponding to a specific recombination site of the Cre-recombinase at the locus of the deleted mic1 gene (site A) when heterologous DNA is inserted at the locus of the deleted mic1 gene or corresponding to a specific recombination site of Cre-recombinase at the locus of the deleted mic3 gene (site B) when heterologous DNA is inserted at the locus of the deleted mic3 gene, or corresponding to a specific recombination site of Cre-recombinase at the locus of the deleted rop16I gene (site D) when heterologous DNA is inserted at the locus of the deleted rop16I gene
site D corresponding to said specific recombination site of the Cre-recombinase at the locus of said deleted rop16I gene
such as, when heterologous DNA is inserted at the locus of the deleted mic1 gene,
said site A corresponds to a LoxN site of SEQ ID NO: 5
said site B corresponds to a LoxP site of SEQ ID NO: 12
said site C corresponds to a LoxN site of SEQ ID NO: 5
said site D corresponds to a Lox2272 site of SEQ ID NO: 68;
or such as when heterologous DNA is inserted at the locus of the deleted mica gene,
said site A corresponds to a LoxN site of SEQ ID NO: 5
said site B corresponds to a LoxP site of SEQ ID NO: 12
said site C corresponds to a LoxP site of SEQ ID NO: 12
said site D corresponds to a Lox2272 site of SEQ ID NO: 68;
or such as when heterologous DNA is inserted at the locus of the deleted rop16I gene,
said site A corresponds to a LoxN site of SEQ ID NO: 5
said site B corresponds to a LoxP site of SEQ ID NO: 12
said site C corresponds to a Lox2272 site of SEQ ID NO: 68
said site D corresponds to a Lox2272 site of SEQ ID NO: 68.
